(19) United States
(12) Patent Application Publication
(10) Pub. No.: US 2026/0091114 A1
(43) Pub. Date: Apr. 2, 2026

(54) CHIMERIC ANTIGEN RECEPTORS BINDING NECTIN-4

(71) Applicant: Fred Hutchinson Cancer Center, Seattle, WA (US)

(72) Inventors: John K. Lee, Seattle, WA (US); Li-Ting Wu, Seattle, WA (US)

(73) Assignee: Fred Hutchinson Cancer Center, Seattle, WA (US)

(21) Appl. No.: 19/112,145

(22) PCT Filed: Sep. 14, 2023
(86) PCT No.: PCT/US2023/074229

§ 371 Date: Mar. 14, 2025

Related U.S. Application Data
(60) Provisional application No. 63/375,642, filed on Sep. 14, 2022.

(51) Int. Cl. A61K 40/42; A61K 40/11; A61K 40/31; C07K 14/725; C07K 16/28

(52) U.S. Cl. CPC A61K 40/4202; A61K 40/11; A61K 40/31; C07K 14/7051; C07K 16/2803; C07K 2317/622

(57) ABSTRACT

Chimeric antigen receptors (CAR) with a binding domain that binds Nectin-4 are disclosed. The Nectin-4 binding domain can be derived from the murine antibody M22-321b41.1. The CAR disclosed herein can be expressed by an immune cell to be used in the treatment of Nectin-4-expressing cancers, such as bladder, breast, ovarian, esophageal, lung, colorectal, or pancreatic cancers.

### CROSS-REFERENCE TO RELATED APPLICATIONS

This application is a U.S. National Phase Patent Application based on International Patent Application No. PCT/US2023/074229, filed on Sep. 14, 2023, which claims priority to U.S. Provisional Patent Application No. 63/375,642, filed on Sep. 14, 2022, the contents of both of which are incorporated herein in their entirety.

### STATEMENT REGARDING SEQUENCE LISTING

The Sequence Listing associated with this application is provided in XML format in lieu of a paper copy and is hereby incorporated by reference into the specification. The name of the XML file containing the Sequence Listing is 3E02872.XML. The XML file is 181,388 bytes, was created on Feb. 27, 2025, and is being submitted electronically via Patent Center.

### FIELD OF THE DISCLOSURE

The current disclosure provides chimeric antigen receptors (CAR) with binding domains that bind Nectin-4. The CAR disclosed herein can be used in the treatment of Nectin-4-expressing cancers, such as bladder cancer (e.g., urothelial carcinoma).

### BACKGROUND OF THE DISCLOSURE

According to the World Health Organization, cancer is a leading cause of death globally, and was responsible for nearly 10 million deaths in 2020. Traditional treatments such as surgery, chemotherapy, and/or radiation therapy have been around for more than a century. In recent years, more targeted therapies have emerged.

Targeted therapies specifically target cancer cells by identifying and exploiting molecular and/or immunophenotypic changes seen primarily in those cells. For example, many cancer cells preferentially express particular markers on their cellular surfaces which may be used as targets for antibody-based therapeutics.

Some targeted therapies use genetically engineered immune system cells such as chimeric antigen receptor (CAR) T-cells to target and kill unwanted cell types, such as cancer cells. CAR are proteins including several distinct subcomponents that allow the genetically modified T-cells to recognize and kill cancer cells. The subcomponents of the CAR proteins include at least an extracellular component and an intracellular component expressed as a single protein or in a form that assembles into a functional unit. The extracellular component includes a binding domain that specifically binds a marker (e.g., an antigen) that is preferentially present on the surface of unwanted cells such as cancer cells. When the binding domain binds such markers, the intracellular component signals the T-cell to destroy the bound cell. CAR can additionally include a transmembrane domain that can link the extracellular component to the intracellular component.

Other subcomponents of the CAR proteins can also be used to increase CAR function. For example, spacers provide CAR with additional conformational flexibility, often increasing the binding domain's ability to bind the targeted cell marker, leading to enhanced cytolytic effects. The appropriate length of a spacer within a particular CAR can depend on numerous factors including how close or far a targeted marker is located from the surface of an unwanted cell's membrane.

Although CAR T-cells have had substantial success in treating various cancers, challenges remain. For example, CAR T-cells can have suboptimal proliferation, functional anergy or exhaustion, impaired memory differentiation, and/or limited persistence, limiting their ability to create or maintain remission in patients. There may additionally be targeted therapy resistance that needs to be addressed. Thus, strategies to improve CAR T-cell efficacy are needed, especially for scenarios with prolonged antigen exposure that can lead to functional anergy and exhaustion.

### SUMMARY OF THE DISCLOSURE

Nectin-4, also known as poliovirus receptor-related-4 or PVRL4, is a single pass type I transmembrane protein of the immunoglobulin family. Nectin-4 belongs to the Nectin family of cellular adhesion molecules which mediate Ca2+-independent cell-cell adhesion at adherens junctions via both homophilic interaction where one Nectin-4 interacts with another Nectin-4, and heterophilic interactions where Nectin-4 interacts with another Nectin family protein such as Nectin-1, Nectin-2, or Nectin-3.

Nectin-4 has been found to be overexpressed in multiple human cancers including bladder cancer (e.g., urothelial carcinoma), breast cancer, ovarian cancer, esophageal cancer, lung cancer, colorectal cancer, head and neck squamous cell cancer, and pancreatic cancer.

The current disclosure provides chimeric antigen receptors (CAR) that bind Nectin-4 for the treatment of Nectin-4-expressing cancers. In particular embodiments, the disclosed CAR include, when expressed by a cell, (i) an extracellular component including a binding domain that binds Nectin-4 and a spacer; (ii) an intracellular component; and (iii) a transmembrane domain linking the extracellular component to the intracellular component. In particular embodiments, the binding domain is a single chain variable fragment (scFv) derived from the murine antibody M22-321b41.1. In particular embodiments, the binding domain derived from the murine antibody M22-321b41.1 is humanized. In particular embodiments, the spacer includes a short spacer, an intermediate spacer, or a long spacer. In particular embodiments, the intracellular component includes a 4-1BB and CD3ζ signaling domains. In particular embodiments, the intracellular component includes CD28, 4-1BB, and CD3ζ signaling domains.

The CAR disclosed herein can be expressed by an immune cell to treat Nectin-4-expressing cancers, such as bladder, breast, ovarian, esophageal, lung, colorectal, or pancreatic cancers. In particular embodiments, for bladder cancer, CAR-expressing cell formulations and/or modifying formulations can be administered by intravesical administration.

### BRIEF DESCRIPTION OF THE FIGURES

Some of the drawings submitted herewith may be better understood in color. Applicant considers the color versions of the drawings as part of the original submission and reserves the right to present color images of the drawings in later proceedings.

FIGS. 1A, 1B. (FIG. 1A) Nectin-4 is a single pass type I transmembrane protein that acts in cell-cell adhesion with other Nectin family members. (FIG. 1B) Nectin-4 expression in cancer is associated with poor progression and relapse.

FIG. 2. Limited normal tissue expression of Nectin-4. Adverse events associated with the Nectin-4 ADC enfortumab vedotin include on-target cutaneous, gastrointestinal (GI), and corneal toxicities. The x-axis reads from left to right: Adipose-Subcutaneous, Adipose-Visceral (Omentum), Adrenal Gland, Artery-Aorta, Artery-Coronary, Artery-Tibial, Bladder, Brain-Amygdala, Brain-Anterior cingulate cortex (B24), Brain-Caudate (basal ganglia), Brain-Cerebellar Hemisphere, Brain-Cerebellum, Brain-Cortex, Brain-Frontal Cortex (BA9), Brain-Hippocampus, Brain-Hypothalamus, Brain-Nucleus accumbens (basal ganglia), Brain-Putamen (basal ganglia), Brain-Spinal cord (cervical c-1), Brain-Substantia nigra, Breast-Mammary Tissue, Cells-Cultured fibroblasts, Cells-EBV-transformed lymphocytes, Cervix-Ectocervix, Cervix-Endocervix, Colon-Sigmoid, Colon-Transverse, Esophagus-Gastroesophageal Junction, Esophagus-Mucosa, Esophagus-Muscularis, Fallopian Tube-, Heart-Atrial Appendage, Heart-Left Ventricle, Kidney-Cortex, Kidney-Medulla, Liver, Lung, Minor Salivary Gland, Muscle-Skeletal, Nerve-Tibial, Ovary, Pancreas, Pituitary, Prostate, Skin-Not Sun Exposed (Suprapubic), Skin-Sun Exposed (Lower Leg), Small Intestine-Terminal Ileum, Spleen, Stomach, Testis, Thyroid, Uterus, Vagina, and Whole Blood.

FIG. 3. Nectin-4 is highly expressed in urothelial carcinoma. EV-101 study, n=152 with metastatic urothelial cancer (UC). Mean H-score (maximum 300) of Nectin-4 staining was 240. Late-stage clinical trials of enfortumab vedotin and its approval were not contingent on selection based on Nectin-4 immunohistochemistry (IHC).

FIG. 4. Schematic of Nectin-4 CAR. Cloning of CAR based on the Nectin-4 antibody clone M22-321b41.1 (see US20200231670). M22-321b41.1 is a mouse IgG2a kappa monoclonal antibody with specificity for human Nectin-4 and is the clone developed/used by Seagen (Bothell, WA) for IHC studies associated with enfortumab vedotin clinical trials.

FIGS. 5A-5H. Efficient lentiviral transduction of CD4T-cells (FIG. 5A-FIG. 5D) and CD8 T-cells (FIG. 5E-FIG. 5H) with M22-321b41.1-BBζ CAR. As used herein, the M22-321b41.1-BBζ CAR includes a GM-CSF signal peptide, M22-321b41.1 VL-VH scFv, long spacer, CD28 transmembrane domain, 4-1BB and CD3z signaling domains, T2A, and EGFRt (SEQ ID NO: 1).

FIG. 6. Generation of isogenic UM-UC-3 and UM-UC-3-Nectin-4 target cell lines to test the specificity of Nectin-4 CAR T-cells. Nectin-4-negative UM-UC-3 cell line transduced with either GFP or Nectin-4 GFP lentivirus. Nectin-4 expression validated by immunoblot and flow cytometry.

FIGS. 7A, 7B, 7C, and 7D. Co-cultures of T-cells with UM-UC-3 Nectin-4 GFP cells (FIG. 7A). Relative viability of untransduced cells at varying effector to target (E:T) ratios. Relative viability of different length spacers (long, medium (also referred to as intermediate, herein), or short) at E:T of 1:1 (FIG. 7B), 1:3 (FIG. 7C), and 1:10 (FIG. 7D). Long spacer M22-321b41.1-BBζ CAR T-cells demonstrate the most pronounced cytolytic activity in co-cultures.

FIG. 8A, 8B. Co-culture killing assays using the UM-UC-9 cell line across dilutions of effector:target ratios indicating that short spacer (IgG4 hinge).

FIGS. 9A, 9B. Assessment of Nectin-4 expression in human urothelial carcinoma cell lines by flow cytometry. Relative median fluorescence intensity (MFI) is shown for each cell line. MFI of labeled cell lines is marked with an asterisk.

FIG. 10. Antigen-specific T-cell activation across co-cultures with human urothelial carcinoma cell lines. Co-cultures at effector:target ratio of 1, supernatant collected at 48 hours. T-cells reconstituted at a CD4/CD8 ratio of 1.

FIG. 11. In vivo antitumor activity of M22-321b41.1-BBζ CAR T-cells. UM-UC-9 subcutaneous tumors established in NOD scid gamma (NSG) mice injected intratumorally with T-cells.

FIG. 12A, 12B. Co-culture killing assays using the UM-UC-9 and UM-UC-9 NECTIN4 knockout cell lines at an effector:target ratio of 1 indicates antigen-specificity of NECTIN4 CAR T cells.

FIG. 13A, 13B. Co-culture killing assays using the UM-UC-9 and UM-UC-9 NECTIN4 knockout cell lines at an effector:target ratio of 1 indicates antigen-specificity of NECTIN4 CAR T cells and that NECTIN4 28ζ CAR T cells are reactive against both human NECTIN4 (FIG. 13A) and mouse Nectin4 (FIG. 13B).

FIG. 14 Robust antitumor activity of NECTIN4 28ζ CAR T cell therapy seen in UM-UC-9 subcutaneous tumors established in NSG mice treated with a single intravenous injection of 5×10⁶ cells.

FIG. 15. Exemplary alternative configurations of M22-321b41.1-BBζ CAR includes swapping the orientation of the scFv and enhancing signaling through the incorporation of both CD28 and 4-1BB costimulatory domains (Nectin-4 scFv from enfortumab vedotin Ha22-2(2,4)6.1).

FIG. 16. Sequences supporting the disclosure. pCCLc-MNDU3-Nectin4-IgG4Hinge-CH2-CH3-CD28TM-41BBSig-CD3zeta-EGFRt Coding Sequence (SEQ ID NO: 1) where AmpR is at residues 330-989; ColE1 origin is at residues 1087-1769; LacO is at residues 2109-2131; M13-rev is at residues 2137-2157; T3 is at residues 2175-2194; CMV promoter is at residues 2362-2890; R is at residues 2891-2986; U5 is at residues 2987-3071; Psi is at residues 3123-3260; RRE is at residues 3725-3924; cPPT is at residues 4469-4648; MNDU3 promoter is at residues 4659-5194; pCCL-F is at residues 5117-5136; Primer CD19R F1 is at residues 5180-5199; EcoR1 is at residues 5208-5213; Kozak is at residues 5220-5228; GM-CSF signal peptide is at residues 5229-5294; M22-321b41.1 VL is at residues 5328-5666; G4Sx3 Linker is at residues 5667-5711; M22-321b41.1 VH is at residues 5712-6059; IgG4 hinge is at residues 6060-6095; 4/2N/Q-1 is at residues 6102-6110; IgG4-CH2 is at residues 6111-6421; 4/2NQ-2 is at residues 6291-6293; IgG4-CH3 is at residues 6421-6731; CD28tm is at residues 6744-6827; 4-1BB is at residues 6828-6953; CD3zeta is at residues 6954-7298; T2A is at residues 7299-7370; EGFRt is at residues 7371-8441; Restored HpaI site is at residues 8445-8450; dWPRE is at residues 8451-9038; UTG-R is at residues 8809-8828; Promoter mutation is at residues 8845-8849; Mutated start codon is at residues 8860-8862; U3 is at residues 9133-9185; R(1) is at residues 9186-9281; U5(1) is at residues 9282-9366; T7 is at residues 9766-9786; M13-fwd is at residues 9795-9812; LacZ alpha is at residues 9883-9951; M13 origin is at residues 9956-10411; and F1 on is at residues 9973-10279; CAR with Nectin-4 binding domain (anti-Nectin4 scFv-IgG4Hinge-CH2-CH3-CD28TM-41BBSig-CD3zeta) (SEQ ID NO: 2); CAR with Nectin-4 binding domain encoding sequence (anti-Nectin4 scFv-IgG4Hinge-CH2-CH3-CD28TM-41BBSig-CD3zeta) (SEQ ID NO: 3); CAR with Nectin-4 binding domain, signal peptide, T2A, and EGFRt (GM-CSF signal peptide-anti-Nectin4 scFv-IgG4Hinge-CH2-CH3-CD28TM-41BBSig-CD3zeta-T2A-EGFRt) (SEQ ID NO: 110); CAR with Nectin-4 binding domain encoding sequence (GM-CSF signal peptide-anti-Nectin4 scFv-IgG4Hinge-CH2-CH3-CD28TM-41BBSig-CD3zeta-T2A-EGFRt) (SEQ ID NO: 111); Nectin-4 [Homo sapiens] (GenBank: BAM65001.1) (SEQ ID NO: 4); AmpR (SEQ ID NO: 5); RRE (SEQ ID NO: 6); cPPT (SEQ ID NO: 7); GM-CSF signal peptide (SEQ ID NO: 8); IgG4 hinge-A (SEQ ID NO: 9); IgG4 hinge coding sequence-A (SEQ ID NO: 10); IgG4 hinge coding sequence-B (SEQ ID NO: 11); IgG4 hinge coding sequence-C (SEQ ID NO: 12); IgG4-CH2 domain-A (SEQ ID NO: 13); IgG4-CH2 domain-B (SEQ ID NO: 14); IgG4-CH2 domain encoding sequence-A (SEQ ID NO: 15); IgG4-CH2 domain encoding sequence-B (SEQ ID NO: 16); IgG4-CH3 domain-A (SEQ ID NO: 17); IgG4 CH3 domain-B (SEQ ID NO: 18); IgG4-CH3 encoding sequence-A (SEQ ID NO: 19); IgG4 CH3 domain coding sequence-B (SEQ ID NO: 20); Intermediate Spacer (IgG4 hinge-CH3 domain) (SEQ ID NO: 21); Intermediate Spacer encoding sequence (IgG4 hinge-CH3 domain) (SEQ ID NO: 22); Long Spacer (IgG4 hinge-CH2 domain-CH3 domain) (SEQ ID NO: 23); Long Spacer encoding sequence (IgG4 hinge-CH2 domain-CH3 domain) (SEQ ID NO: 24); CD28 Transmembrane Domain-A (SEQ ID NO: 25); CD28 Transmembrane Domain-B (SEQ ID NO: 26); CD28 Transmembrane Domain-C (SEQ ID NO: 27); CD28TM encoding sequence-A (SEQ ID NO: 28); CD28TM encoding sequence-B (SEQ ID NO: 29); 41BB costimulatory domain-A (SEQ ID NO: 32); 4-1BB costimulatory domain-B (SEQ ID NO: 33); 4-1BB costimulatory domain-C (SEQ ID NO: 34); 4-1BB signaling encoding sequence-A (SEQ ID NO: 35); 4-1BB signaling encoding sequence-B (SEQ ID NO: 36); 4-1BB signaling coding sequence-C (SEQ ID NO: 37); CD28 signaling domain (SEQ ID NO: 115); CD28 signaling domain coding sequence (SEQ ID NO: 116); CD28/4-1BB signaling domain (SEQ ID NO: 112); CD28/4-1BB signaling domain coding sequence (SEQ ID NO: 113); CD3ζ signaling domain-A (SEQ ID NO: 38); CD3ζ signaling domain-B (SEQ ID NO: 39); CD3ζ signaling domain-C (SEQ ID NO: 40); CD3zeta signaling domain encoding sequence-A (SEQ ID NO: 41); CD3ζ signaling domain encoding sequence-B (SEQ ID NO: 42); Thosea asigna Virus 2A (T2A) Peptide-v1 (SEQ ID NO: 43); Thosea asigna Virus 2A (T2A) Peptide-v2 (SEQ ID NO: 44); Porcine Teschovirus-12a (P2A) Peptide (SEQ ID NO: 45); Equine Rhinitis A Virus (ERAV) 2A (E2A) Peptide (SEQ ID NO: 46); Foot-And-Mouth Disease Virus 2A (F2A) Peptide (SEQ ID NO: 47); T2A encoding sequence-v1 (SEQ ID NO: 48); T2A encoding sequence-v2 (SEQ ID NO: 49); EGFRt (SEQ ID NO: 50); EGFRt encoding sequence (SEQ ID NO: 51); tCD19 coding sequence (SEQ ID NO: 121); tCD19 (SEQ ID NO: 122); CD28/4-1BB signaling domain (SEQ ID NO: 114); CD28 signaling domain (SEQ ID NO: 115); CD28 signaling domain coding sequence (SEQ ID NO: 116); CD28TM encoding sequence-C (SEQ ID NO: 117); CD28TM sequence-C (SEQ ID NO: 118); CD28TM encoding sequence-D (SEQ ID NO: 119); CD28TM sequence-D (SEQ ID NO: 120).

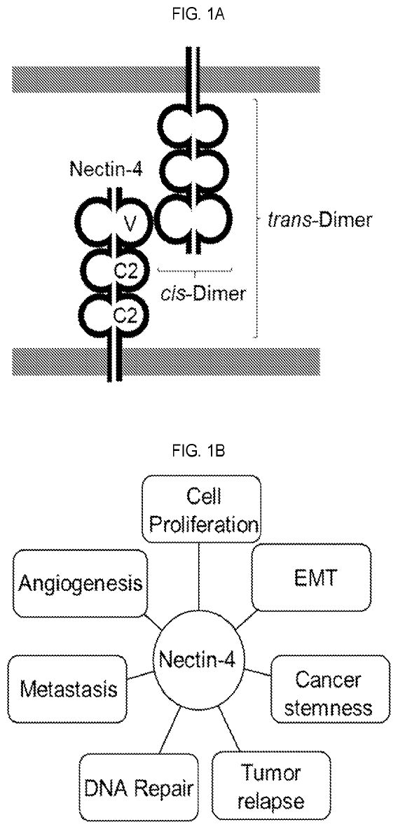

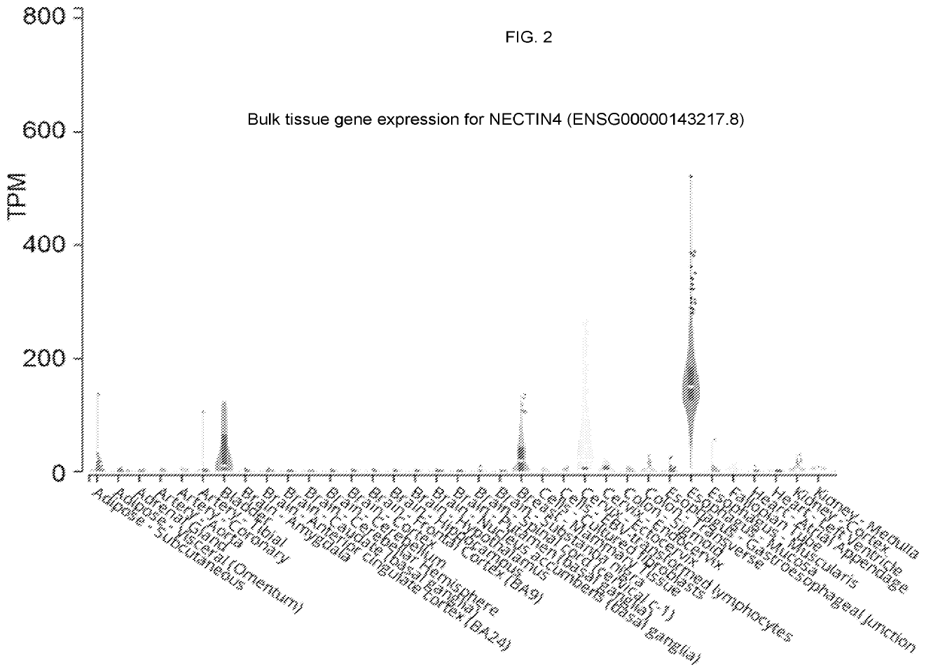

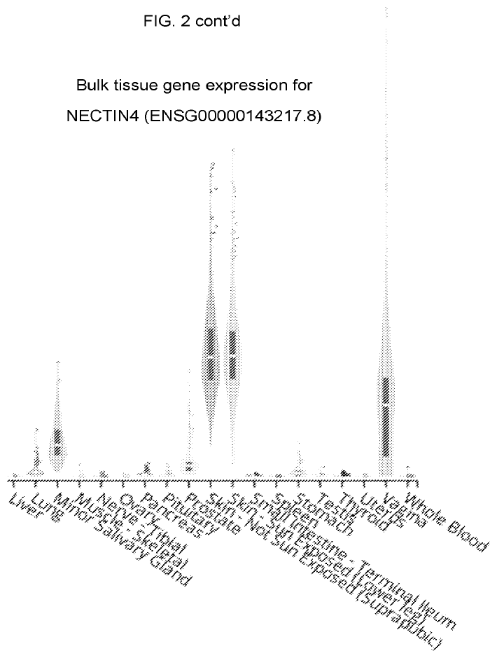

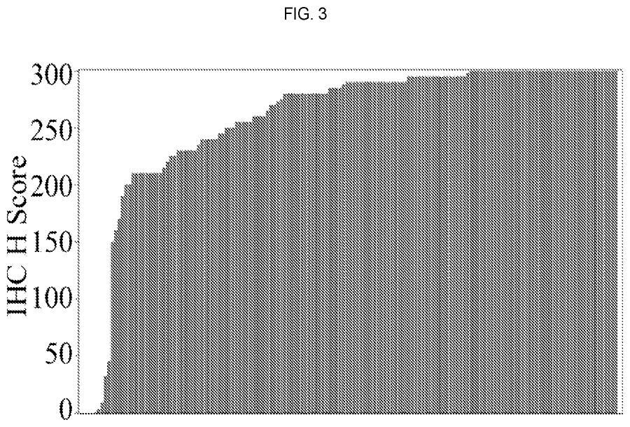

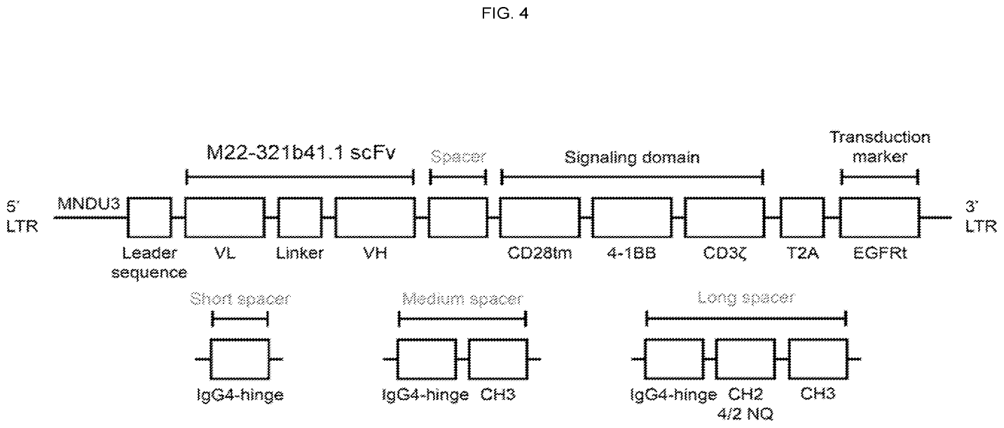

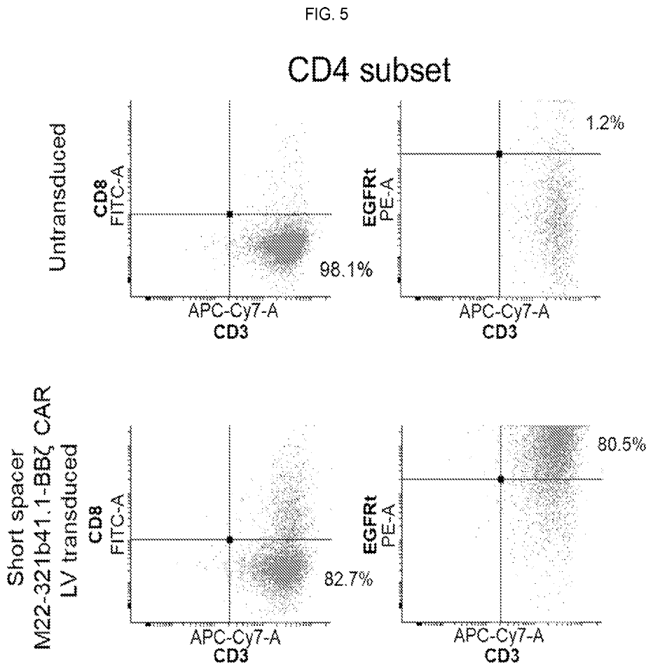

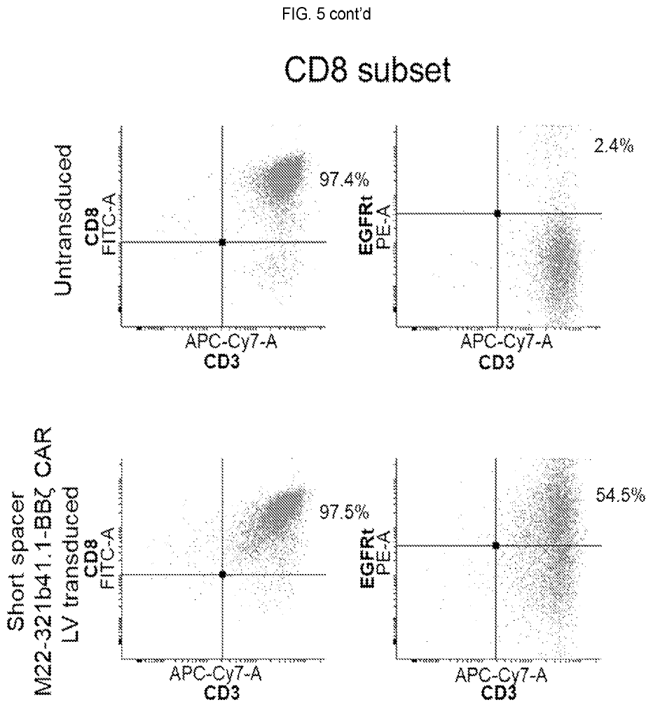

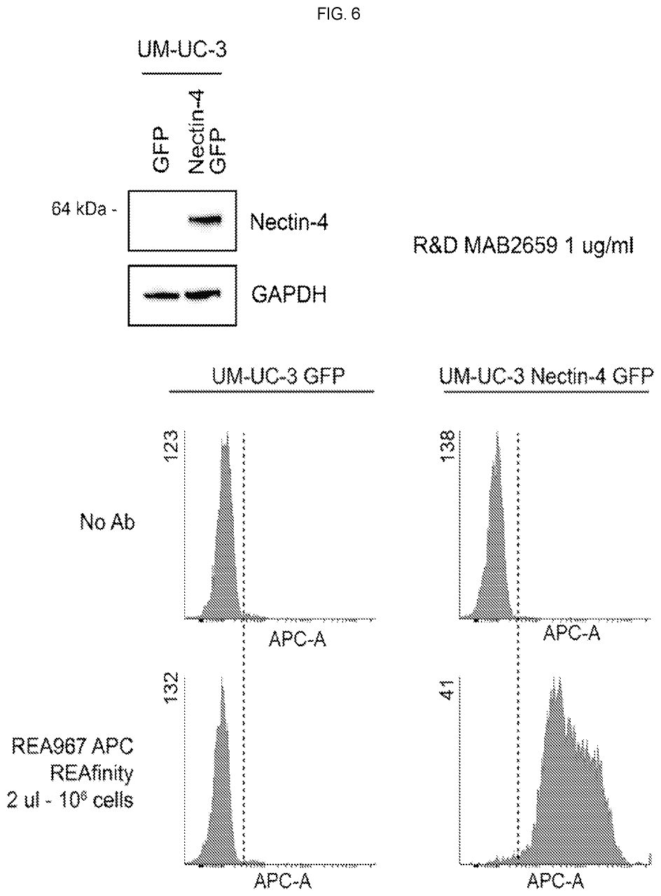

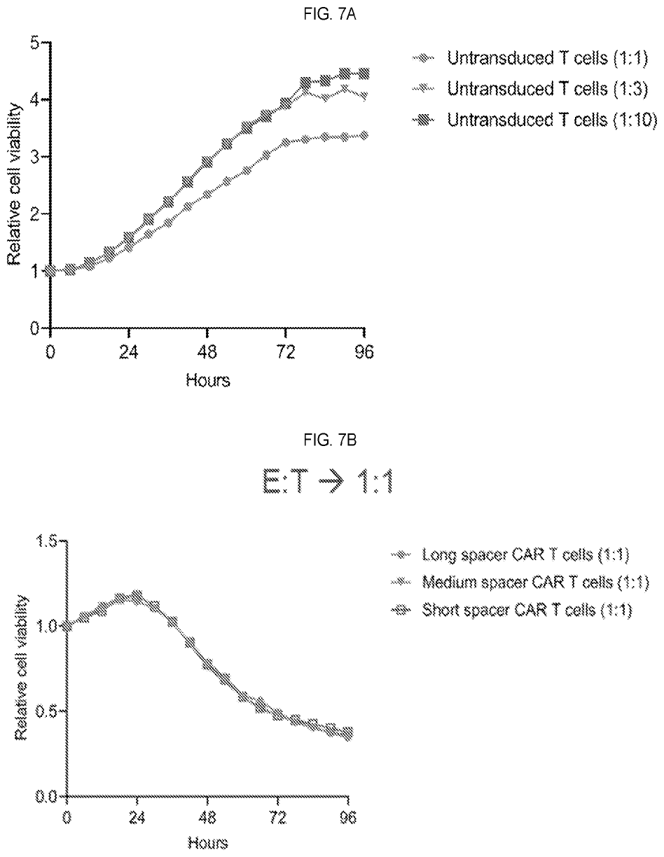

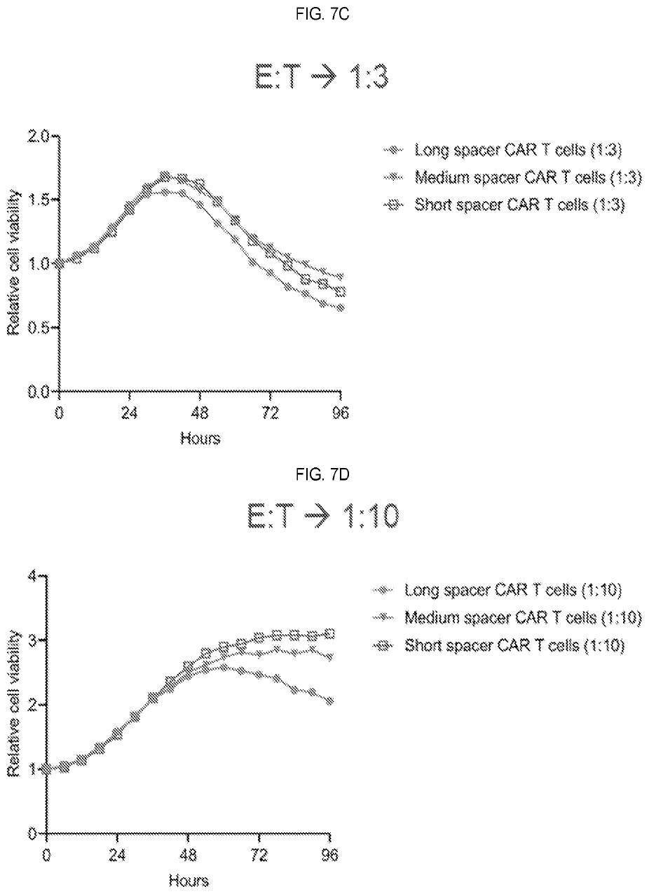

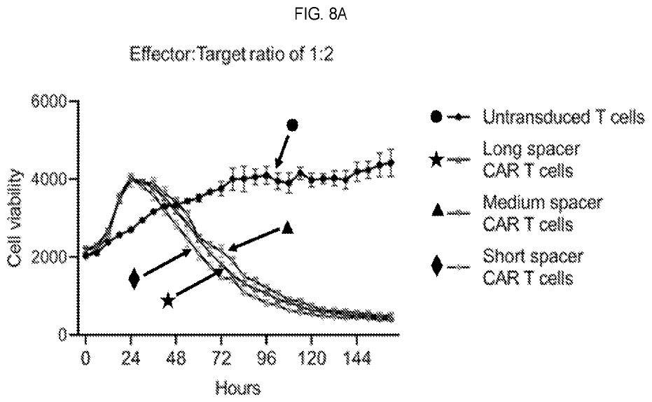

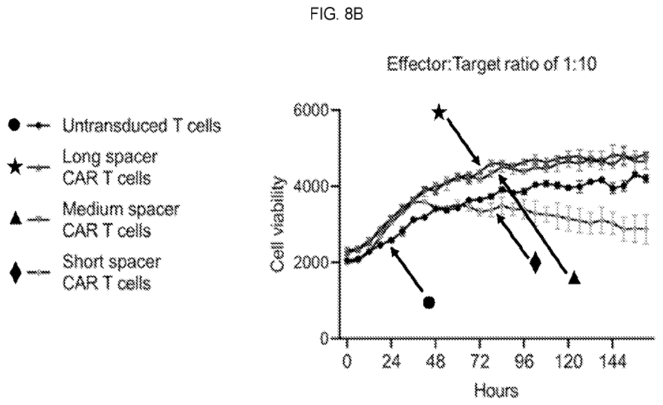

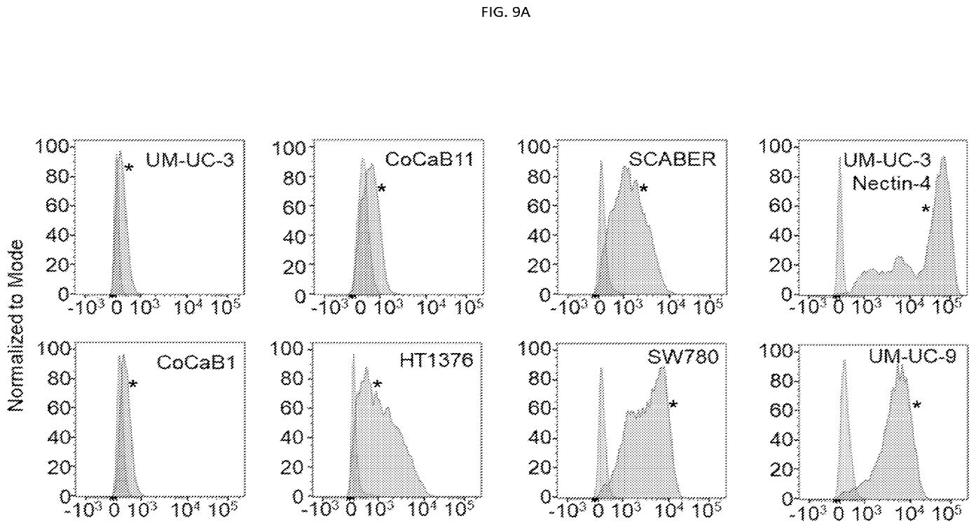

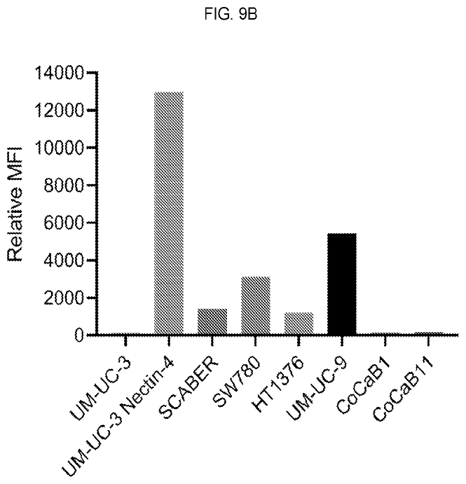

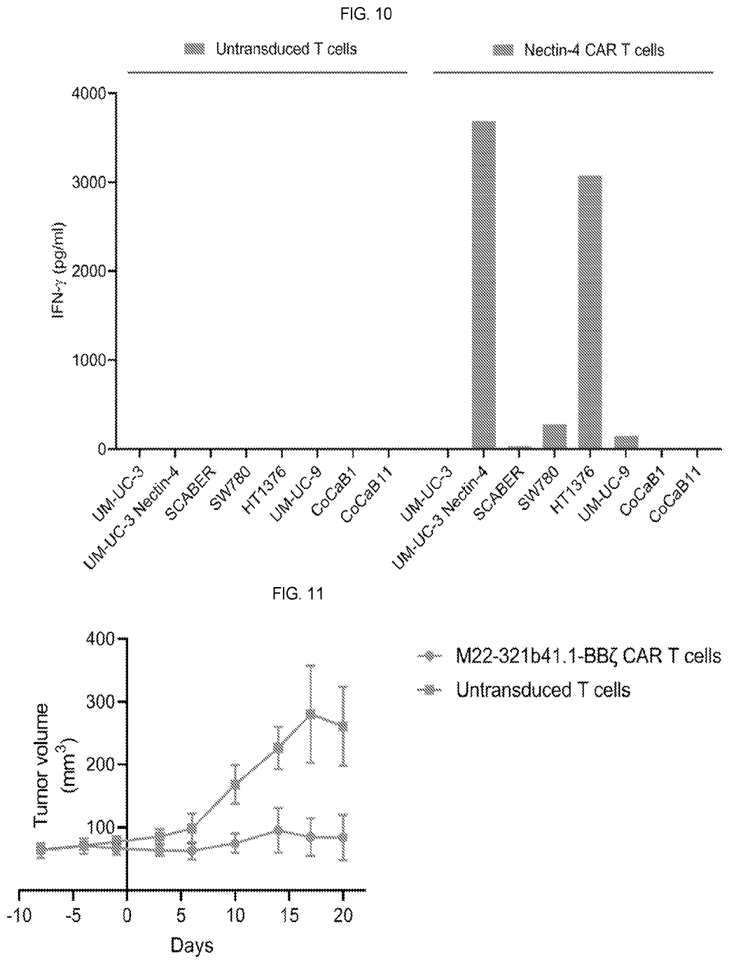

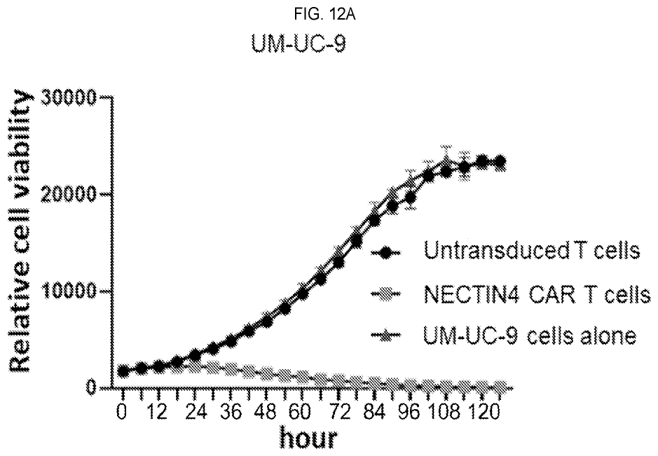

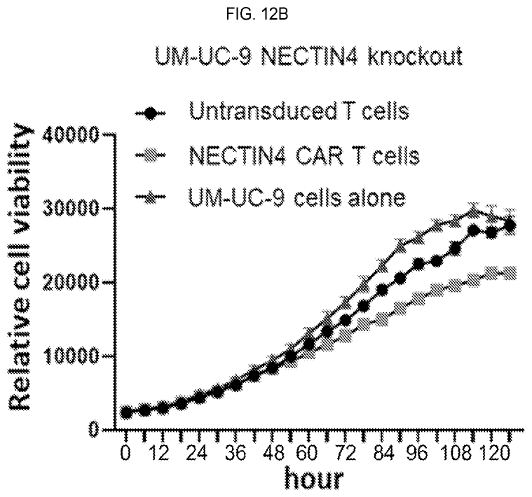

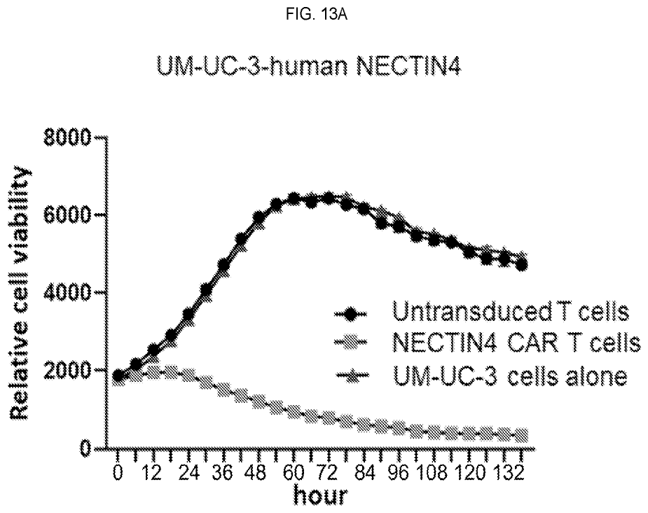

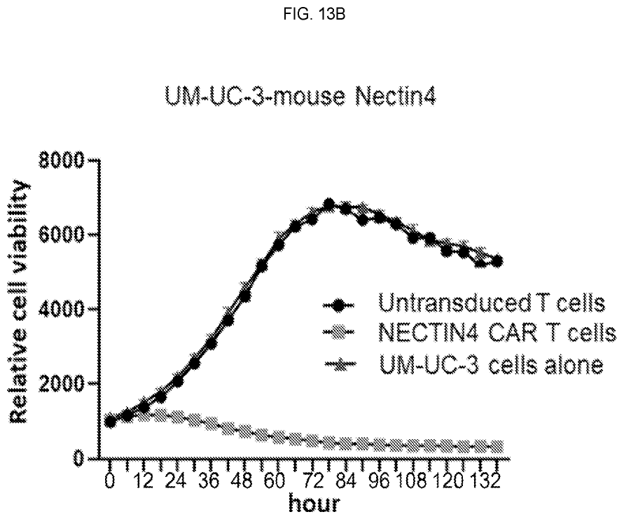

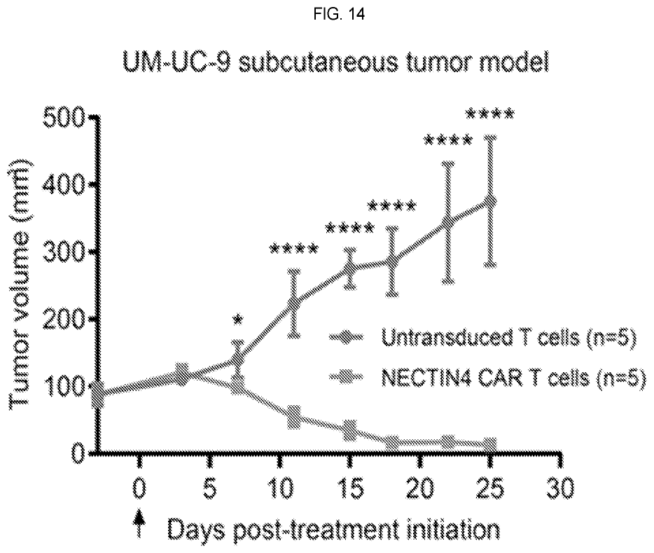

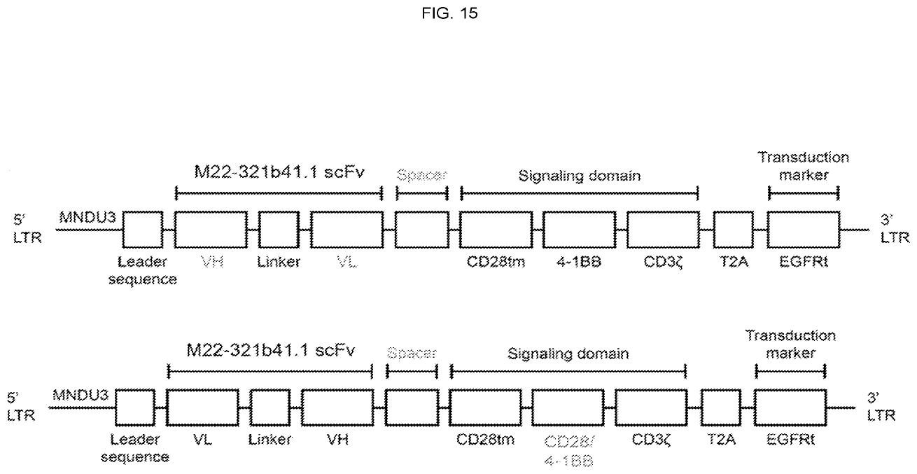

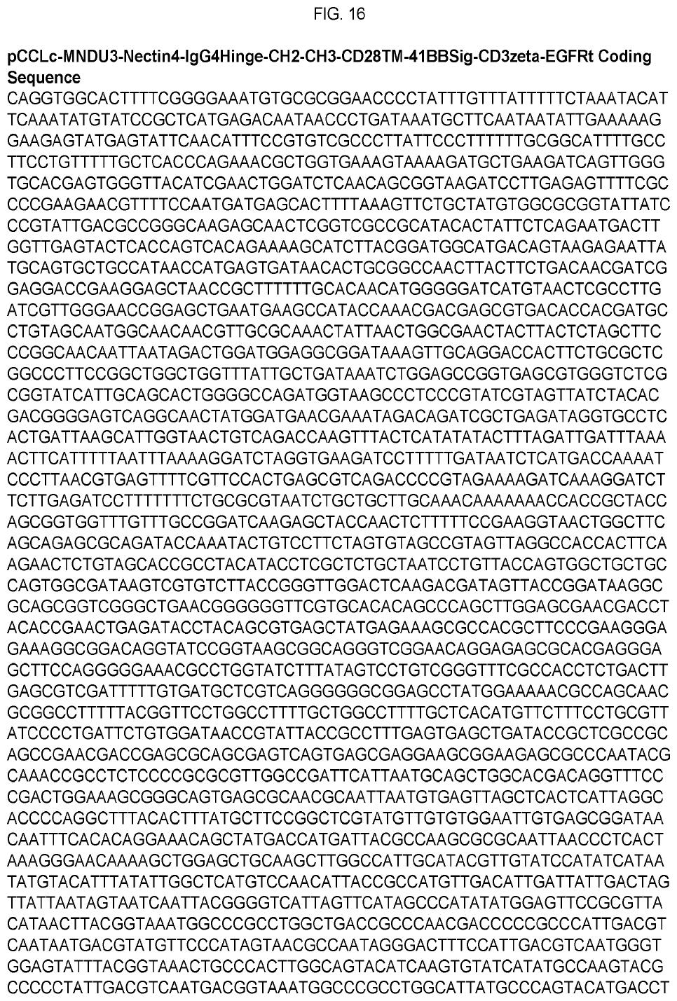

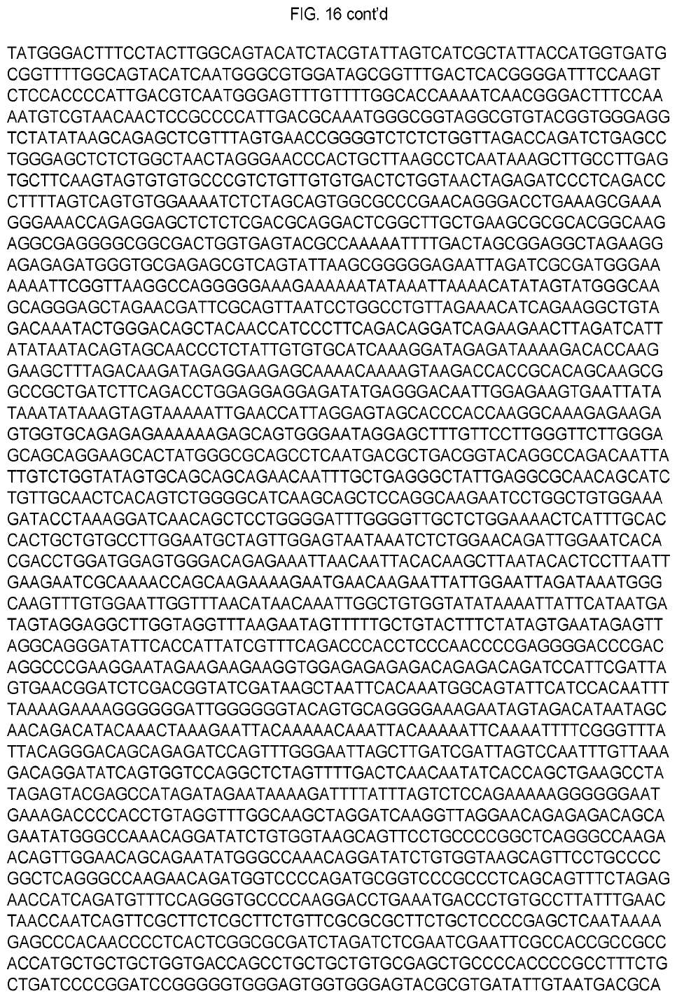

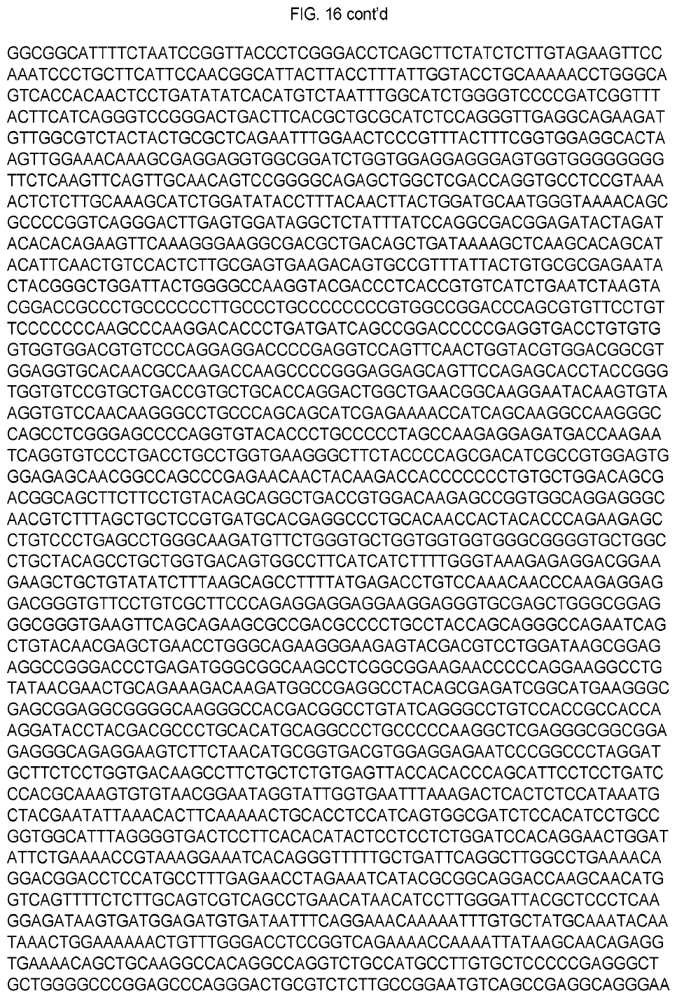

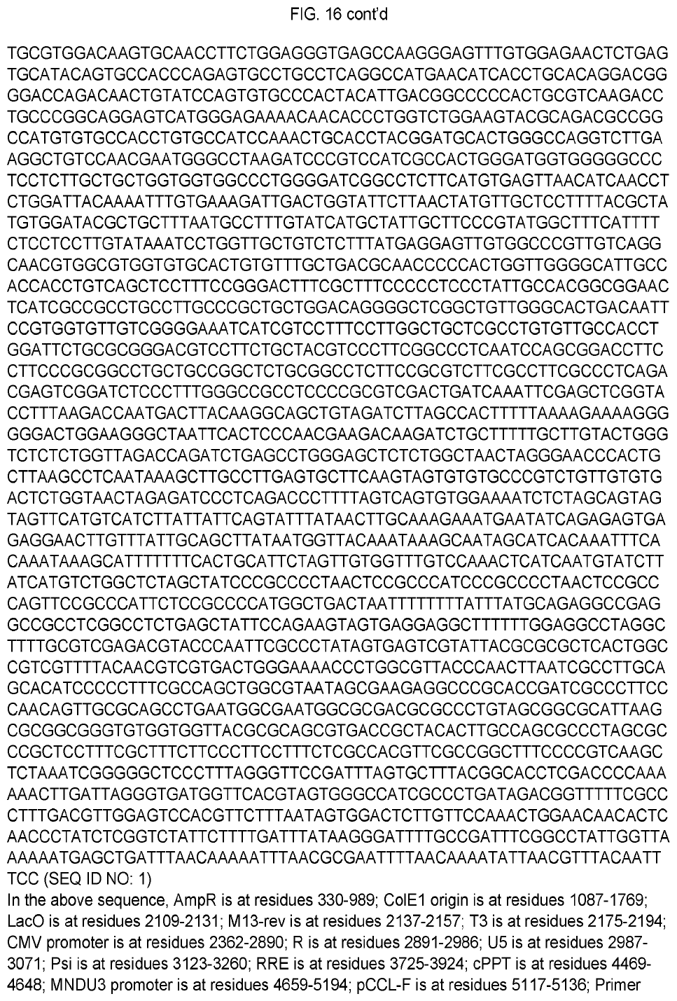

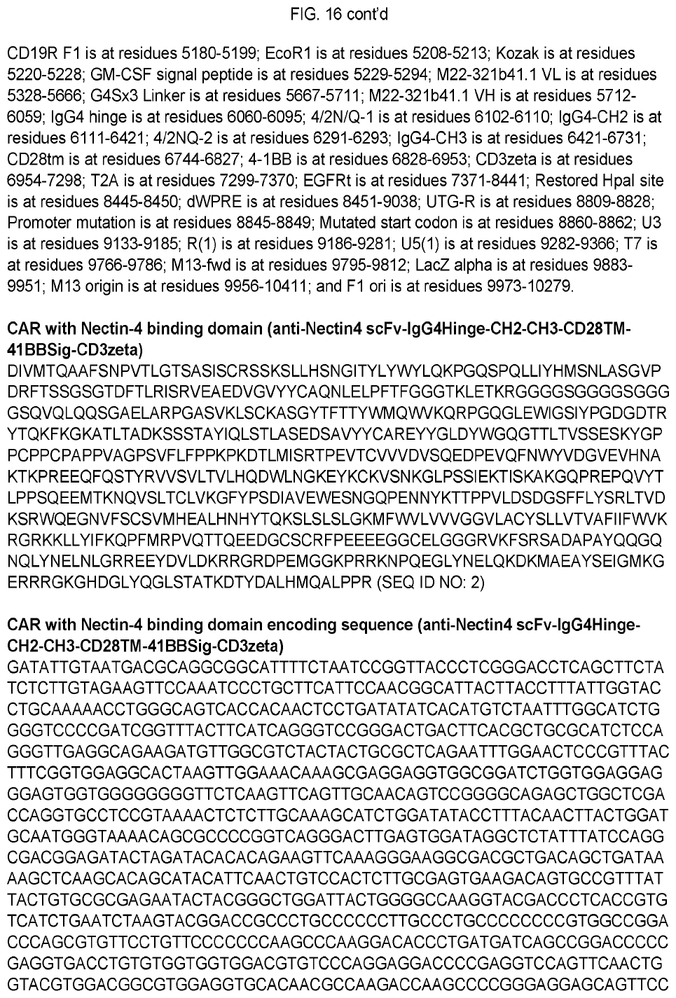

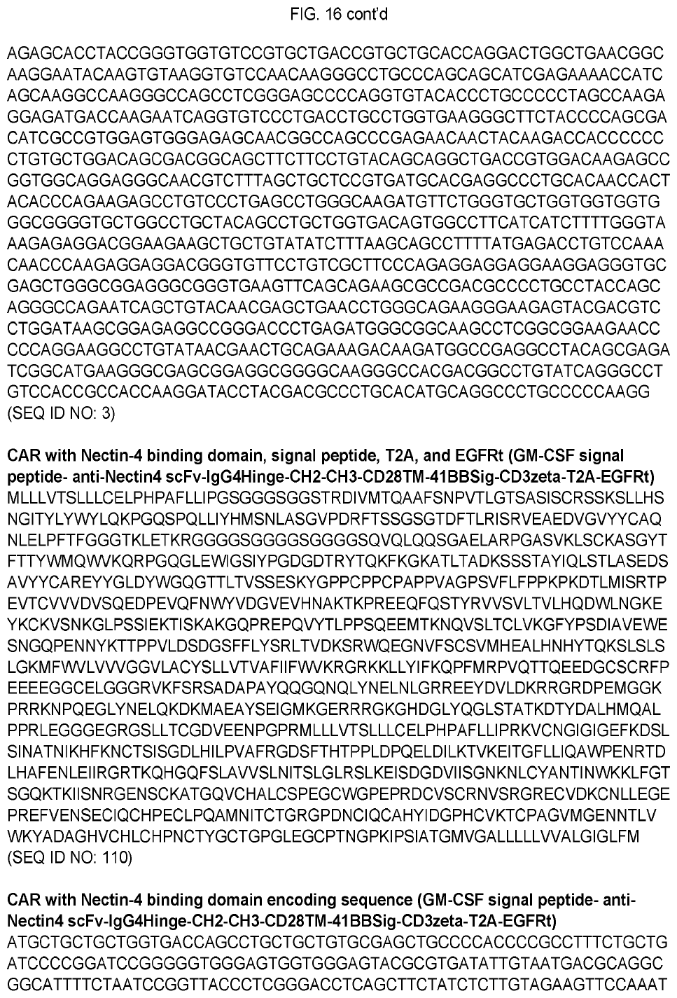

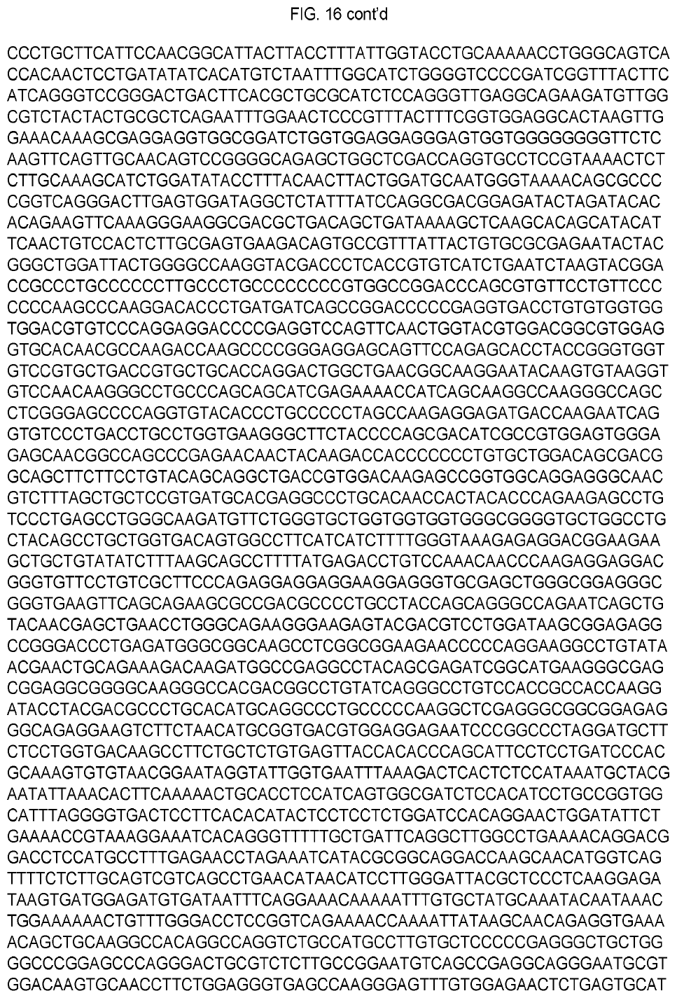

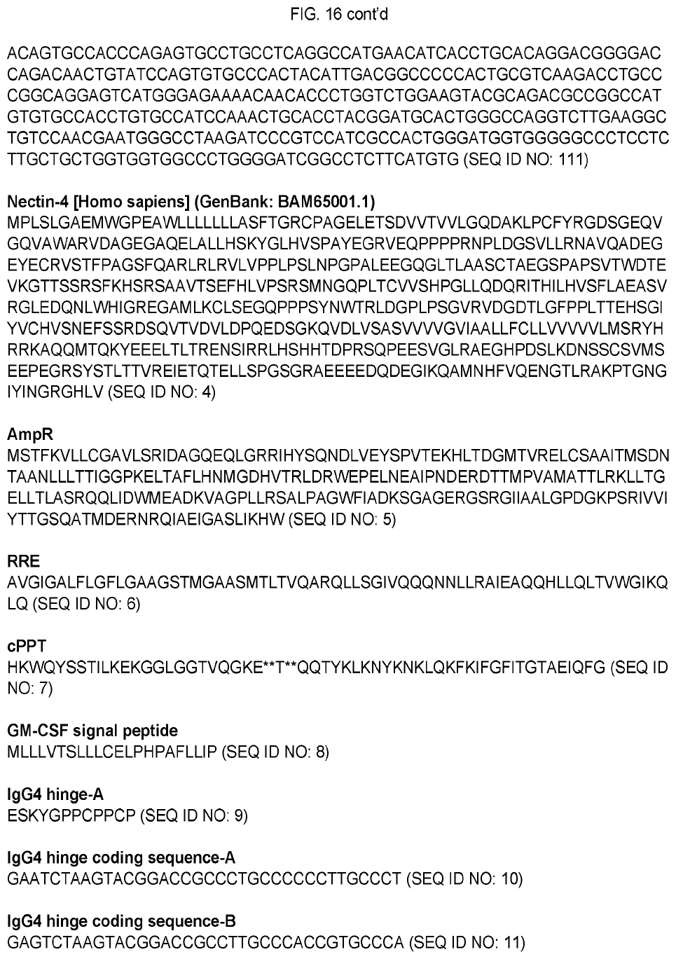

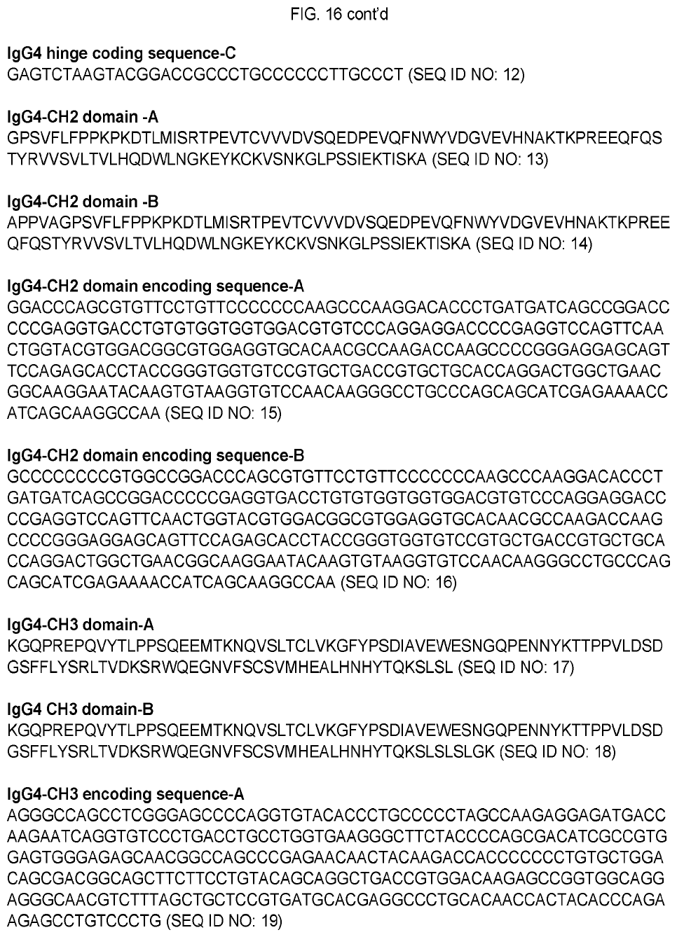

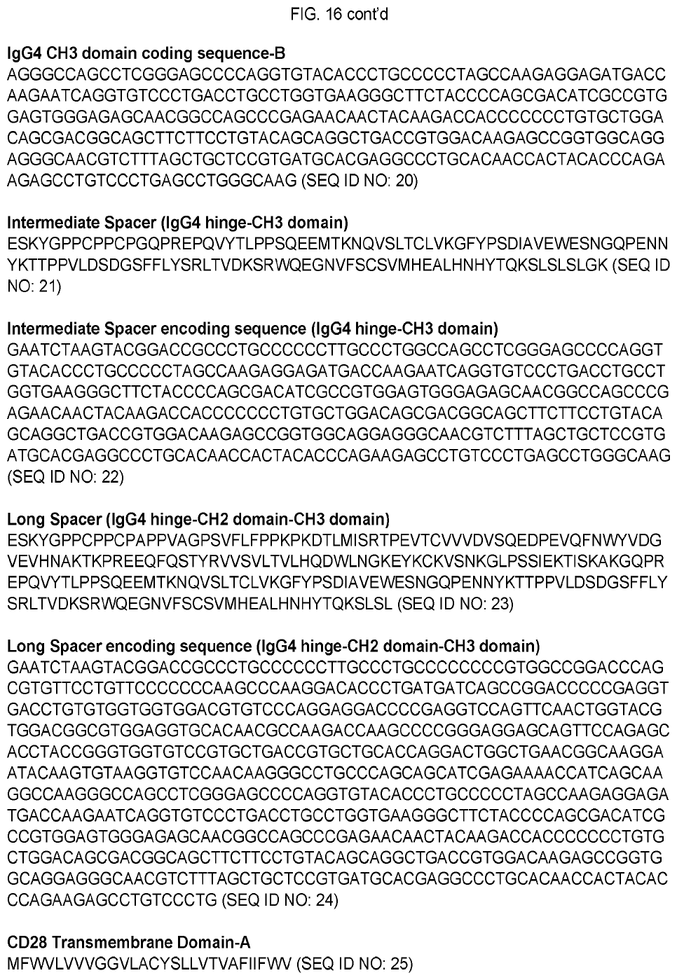

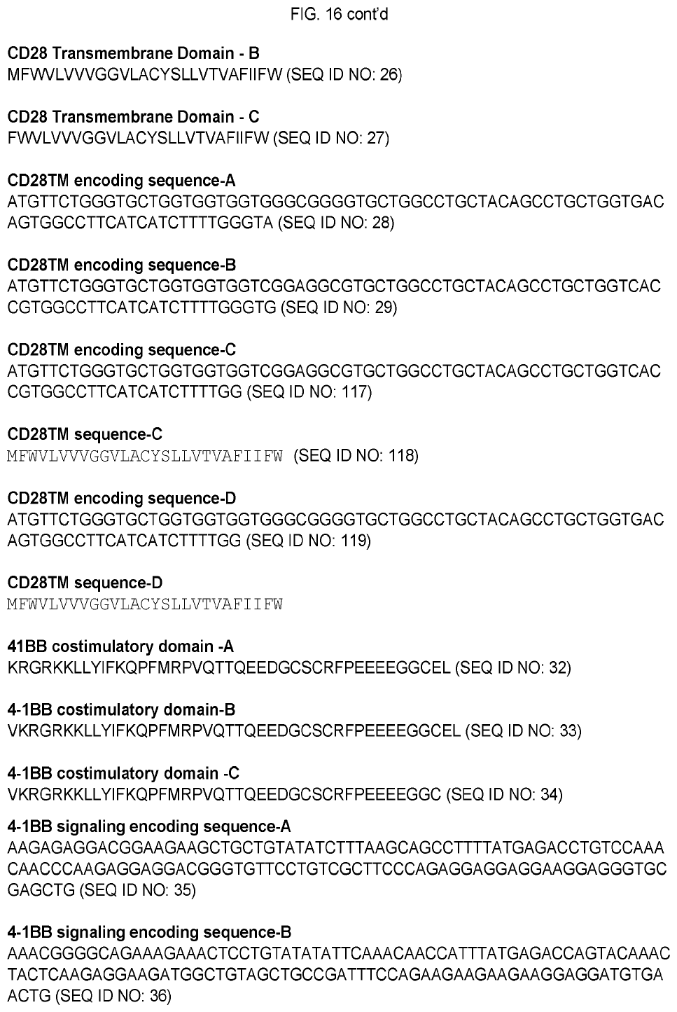

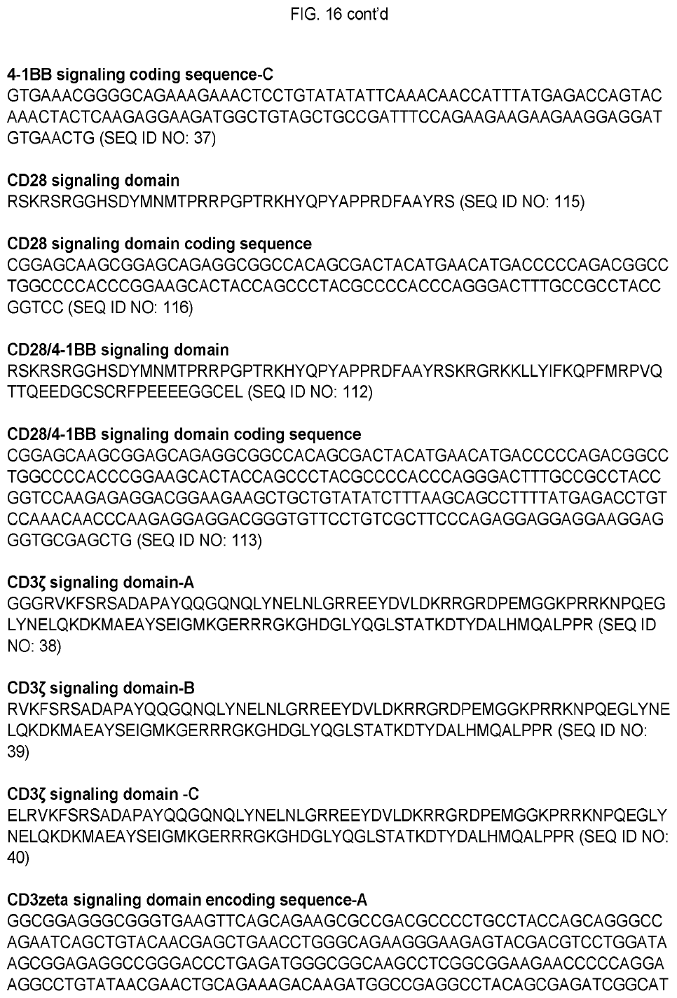

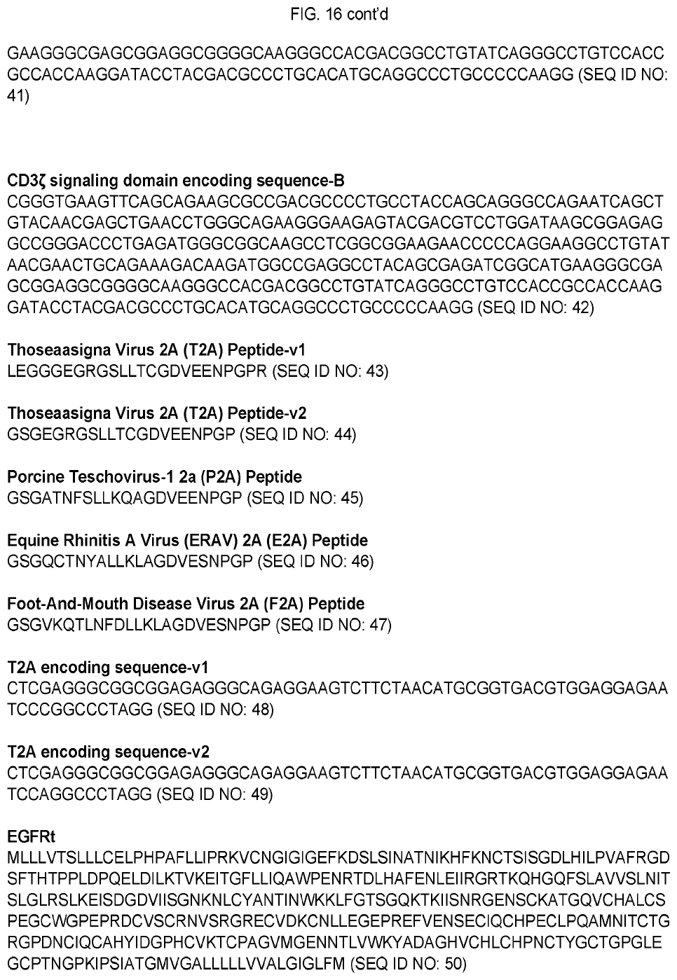

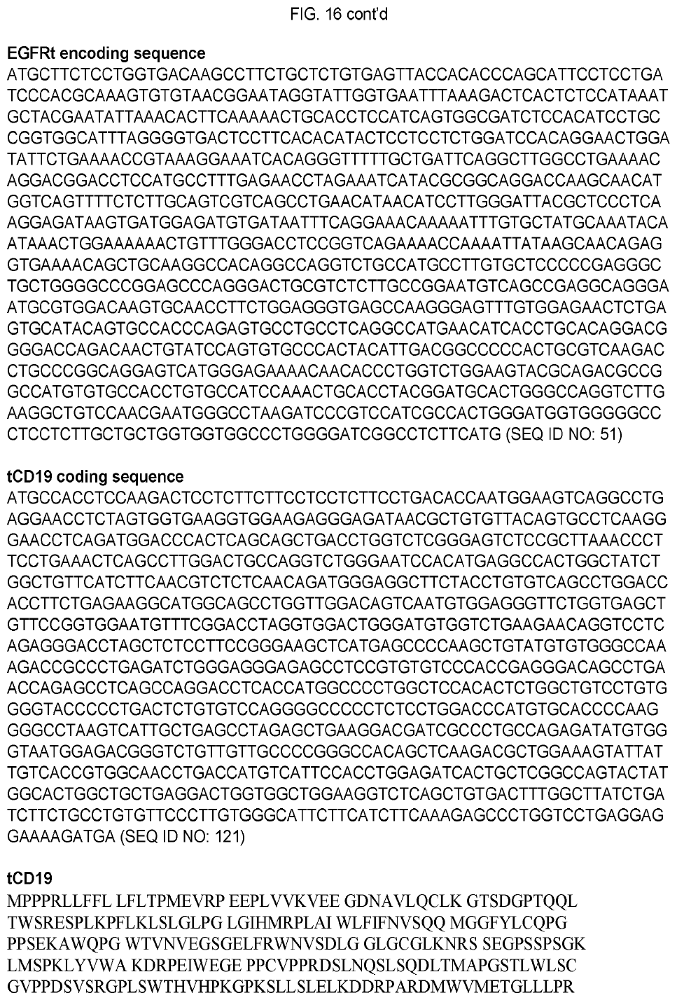

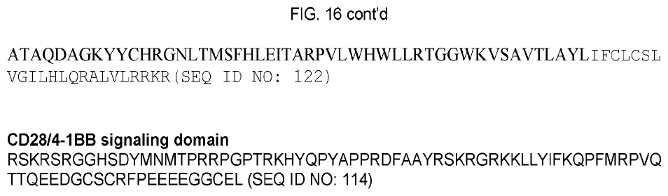

### DETAILED DESCRIPTION

For many years, the chosen treatments for cancer were surgery, chemotherapy, and/or radiation therapy. In recent years, more targeted therapies have emerged to specifically target cancer cells by identifying and exploiting specific molecular and/or immunophenotypic changes seen primarily in those cells. For example, many cancer cells preferentially express particular antigens on their cellular surfaces and these antigens have provided targets for successful therapeutics.

Nectin-4 has been found to be overexpressed in multiple human cancers including bladder cancer, breast cancer, ovarian cancer, esophageal cancer, lung cancer, colorectal cancer, head and neck squamous cell cancer, and pancreatic cancer.

Nectin-4, also known as poliovirus receptor-related-4 or PVRL4, is a single pass type I transmembrane protein of the immunoglobulin family. Nectin-4 belongs to the Nectin family of cellular adhesion molecules which mediate Ca2+-independent cell-cell adhesion at adherens junctions via both homophilic interaction where one Nectin-4 interacts with another Nectin-4, and heterophilic interactions where Nectin-4 interacts with another Nectin family protein such as Nectin-1, Nectin-2, or Nectin-3. (Miyoshi J et al., 2007, Am J Nephrol 27:590-604; Takai et al., 2003, Cancer Sci 94:655-667; Reymond N. et al., 2001, Journal of Biological Chemistry, 276:43205-15). The extracellular domain of Nectin-4 has three Ig-like subdomains designated as V, C1, and C2. The C1 domain in Nectin-2 is responsible for homophilic interactions, while V domains of most Nectin molecules contribute to heterophilic interactions and cell-cell adhesion. (Miyoshi J et al., 2007, Am J Nephrol 27:590-604; Takai et al., 2003, Cancer Sci 94:655-667; Reymond N. et al., 2001, Journal of Biological Chemistry, 276:43205-15).

The current disclosure provides chimeric antigen receptors (CAR) that bind Nectin-4 (also referred to as Nectin-4 CAR) for the treatment of Nectin-4-expressing cancers. In particular embodiments, the disclosed CAR include, when expressed by a cell, (i) an extracellular component that binds Nectin-4 and a spacer; (ii) an intracellular component; and (iii) a transmembrane domain linking the extracellular component to the intracellular component. In particular embodiments, the binding domain is an scFv derived from the murine antibody M22-321b41.1. In particular embodiments, the binding domain is a humanized scFv derived from the murine antibody M22-321b41.1. In particular embodiments, the spacer includes a short spacer (SEQ ID NO: 9), an intermediate spacer (SEQ ID NO: 21), or a long spacer (SEQ ID NO: 23). In particular embodiments, the spacer includes a long spacer (SEQ ID NO: 23). In particular embodiments, the intracellular component includes a 4-1BB and a CD3z signaling domain. In particular embodiments, the intracellular component includes a CD28, a 4-1BB, and a CD3z signaling domain. In particular embodiments, the transmembrane domain includes a CD28 transmembrane domain.

In particular embodiments, a Nectin-4 CAR is encoded by the sequence as set forth in SEQ ID NO: 1, 3, or 111. In particular embodiments, a Nectin-4 CAR includes the sequence as set forth in SEQ ID NO: 2 or 110.

The CAR disclosed herein can be expressed by an immune cell to be used in the treatment of Nectin-4-expressing cancers, such as bladder (e.g., urothelial carcinoma), breast, ovarian, esophageal, lung, colorectal, and pancreatic cancers. In particular embodiments, for bladder cancer, CAR-expressing cell formulations and/or modifying formulations can be administered by intravesical administration.

Aspects of the current disclosure are now described in more supporting detail as follows: (i) Immune Cells; (ii) Cell Sample Collection and Cell Enrichment; (iii) Genetically Modifying Cell Populations to Express Chimeric Antigen Receptors (CAR); (iii-a) Genetic Engineering Techniques; (iii-b) CAR Subcomponents; (iii-b-i) Binding Domains; (iii-b-ii) Spacers; (iii-b-iii) Transmembrane Domains; (iii-b-iv) Intracellular Components; (iii-b-v) Linkers; (iii-b-vi) Control Features Including Tag Cassettes, Transduction Markers, and/or Suicide Switches; (iii-b-vii) Multimerization Domains; (iv) Characterization of Genetically Engineered Cells; (v) Cell Activating Culture Conditions; (vi) Ex Vivo Manufactured Cell Formulations; (vii) Targeted Viral Vectors & Nanoparticles for In Vivo Cell Modification; (viii) Methods of Use; (ix) Kits; (x) Exemplary Embodiments; and (xi) Closing Paragraphs. These headings are provided for organizational purposes only and do not limit the scope or interpretation of the disclosure.

### (i) IMMUNE CELLS

The present disclosure describes cells genetically modified to express CAR. Genetically modified cells can include T-cells, B cells, natural killer (NK) cells, NK-T-cells, monocytes/macrophages, lymphocytes, hematopoietic stem cells (HSCs), hematopoietic progenitor cells (HPC), and/or a mixture of HSC and HPC (i.e., HSPC). In particular embodiments, genetically modified cells include T-cells.

Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γδ T-cells, the TCR is made up of one γ-chain and one δ-chain. This group of T-cells is much less common (2% of total T-cells) than the ap T-cells.

CD3 is expressed on all mature T-cells. Activated T-cells express 4-1BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

“Central memory” T-cells (or “TCM”) refers to an antigen experienced CTL that expresses CD62L or CCR7 and CD45RO on the surface thereof and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells refer to a non-antigen experienced T-cell that expresses CD62L and CD45RA and does not express CD45RO as compared to central or effector memory cells. In particular embodiments, naive CD8+ T lymphocytes are characterized by the expression of phenotypic markers of naive T-cells including CD62L, CCR7, CD28, CD127, and CD45RA.

Natural killer cells (also known as NK cells, K cells, and killer cells) are activated in response to interferons or macrophage-derived cytokines. They contain viral infections while the adaptive immune response is generating antigen-specific cytotoxic T-cells that can clear the infection. NK cells express CD8, CD16 and CD56 but do not express CD3.

NK cells include NK-T-cells. NK-T-cells are a specialized population of T-cells that express a semi invariant T-cell receptor (TCR ab) and surface antigens typically associated with natural killer cells. NK-T-cells contribute to antibacterial and antiviral immune responses and promote tumor-related immunosurveillance or immunosuppression. Like natural killer cells, NK-T-cells can also induce perforin-, Fas-, and TNF-related cytotoxicity. Activated NK-T-cells are capable of producing IFN-γ and IL-4. In particular embodiments, NK-T-cells are CD3+/CD56+.

Macrophages (and their precursors, monocytes) reside in every tissue of the body (in certain instances as microglia, Kupffer cells and osteoclasts) where they engulf apoptotic cells, pathogens and other non-self-components. Monocytes/macrophages express CD11b, F4/80; CD68; CD11c; IL-4Rα; and/or CD163.

Immature dendritic cells (i.e., pre-activation) engulf antigens and other non-self-components in the periphery and subsequently, in activated form, migrate to T-cell areas of lymphoid tissues where they provide antigen presentation to T-cells. Dendritic cells express CD1a, CD1b, CD1c, CD1d, CD21, CD35, CD39, CD40, CD86, CD101, CD148, CD209, and DEC-205.

Hematopoietic Stem/Progenitor Cells or HSPC refer to a combination of hematopoietic stem cells and hematopoietic progenitor cells.

Hematopoietic stem cells refer to undifferentiated hematopoietic cells that are capable of self-renewal either in vivo, essentially unlimited propagation in vitro, and capable of differentiation to all other hematopoietic cell types.

A hematopoietic progenitor cell is a cell derived from hematopoietic stem cells or fetal tissue that is capable of further differentiation into mature cell types. In certain embodiments, hematopoietic progenitor cells are CD24^{lo} Lin⁻ CD117⁺ hematopoietic progenitor cells. HPC can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and NK-cells.

HSPC can be positive for a specific marker expressed in increased levels on HSPC relative to other types of hematopoietic cells. For example, such markers include CD34, CD43, CD45RO, CD45RA, CD59, CD90, CD109, CD117, CD133, CD166, HLA DR, or a combination thereof. Also, the HSPC can be negative for an expressed marker relative to other types of hematopoietic cells. For example, such markers include Lin, CD38, or a combination thereof. Preferably, the HSPC are CD34⁺ cells.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

In particular embodiments, immune cells to be genetically modified according to the teachings of the current disclosure can be patient-derived cells (autologous) or allogeneic. In particular embodiments, immune cells can be genetically modified in vivo or ex vivo.

### (ii) CELL SAMPLE COLLECTION AND CELL ENRICHMENT

Methods of sample collection and enrichment are known by those skilled in the art. In particular embodiments, cells are derived from humans, for example a patient to be treated. Cells can be derived from cell lines.

In some embodiments, T-cells are derived or isolated from samples such as whole blood, peripheral blood mononuclear cells (PBMCs), leukocytes, bone marrow, thymus, tissue biopsy, tumor, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In particular embodiments, cells from the circulating blood of a subject are obtained, e.g., by apheresis or leukapheresis. The samples, in particular embodiments, contain lymphocytes, including T-cells, monocytes, granulocytes, B cells, other nucleated white blood cells, HSC, HPC, HSPC, red blood cells, and/or platelets, and in some aspects contains cells other than red blood cells and platelets and further processing is necessary. In particular embodiments, T-cells are derived from PBMCs.

In some embodiments, blood cells collected from a subject are washed, e.g., to remove the plasma fraction and to place the cells in an appropriate buffer or media for subsequent processing steps.

Isolation can include one or more of various cell preparation and separation steps, including separation based on one or more properties, such as size, density, sensitivity or resistance to particular reagents, and/or affinity, e.g., immunoaffinity, to antibodies or other binding partners. In particular embodiments, the isolation is carried out using the same apparatus or equipment sequentially in a single process stream and/or simultaneously. In particular embodiments, the isolation, culture, and/or engineering of the different populations is carried out from the same starting material, such as from the same sample.

In particular embodiments, a sample can be enriched for T-cells by using density-based cell separation methods and related methods. For example, white blood cells can be separated from other cell types in the peripheral blood by lysing red blood cells and centrifuging the sample through a Percoll or Ficoll gradient.

In particular embodiments, a bulk T-cell population can be used that has not been enriched for a particular T-cell type. In particular embodiments, a selected T-cell type can be enriched for and/or isolated based on cell-marker based positive and/or negative selection. In positive selection, cells having bound cellular markers are retained for further use. In negative selection, cells not bound by a capture agent, such as an antibody to a cellular marker are retained for further use. In some examples, both fractions can be retained for a further use. In particular embodiments, CD4+ and/or CD8+ T-cells are enriched from PBMCs.

The separation need not result in 100% enrichment or removal of a particular cell population or cells expressing a particular marker. For example, positive selection of or enrichment for cells of a particular type refers to increasing the number or percentage of such cells but need not result in a complete absence of cells not expressing the marker. Likewise, negative selection, removal, or depletion of cells of a particular type refers to decreasing the number or percentage of such cells but need not result in a complete removal of all such cells.

In some embodiments, an antibody or binding domain for a cellular marker is bound to a solid support or matrix, such as a magnetic bead or paramagnetic bead, to allow for separation of cells for positive and/or negative selection.

In some embodiments, affinity-based selection is via magnetic-activated cell sorting (MACS) (Miltenyi Biotec, Auburn, CA). MACS systems are capable of high-purity selection of cells having magnetized particles attached thereto.

In some embodiments, a cell population described herein is collected and enriched (or depleted) via flow cytometry, in which cells stained for multiple cell surface markers are carried in a fluidic stream.

Cell-markers for different T-cell subpopulations are described above. In particular embodiments, specific subpopulations of T-cells, such as cells positive or expressing high levels of one or more surface markers, e.g., CCR7, CD45RO, CD8, CD27, CD28, CD62L, CD127, CD4, and/or CD45RA T-cells, are isolated by positive or negative selection techniques.

CD3+, CD28+ T-cells can be positively selected for and expanded using anti-CD3/anti-CD28 conjugated magnetic beads (e.g., DYNABEADS® M-450 CD3/CD28 T Cell Expander).

In particular embodiments, a CD8+ or CD4+ selection step is used to separate CD4+ helper and CD8+ cytotoxic T-cells. Such CD8+ and CD4+ populations can be further sorted into sub-populations by positive or negative selection for markers expressed or expressed to a relatively higher degree on one or more naive, memory, and/or effector T-cell subpopulations.

In some embodiments, enrichment for central memory T (TCM) cells is carried out. In particular embodiments, memory T-cells are present in both CD62L subsets of CD8+ peripheral blood lymphocytes. PBMC can be enriched for or depleted of CD62L, CD8 and/or CD62L+CD8+ fractions, such as by using anti-CD8 and anti-CD62L antibodies.

In some embodiments, the enrichment for central memory T (TCM) cells is based on positive or high surface expression of CCR7, CD45RO, CD27, CD62L, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CCR7, CD45RO, and/or CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L.

Other cell types can be enriched based on known marker profiles and techniques. For example, CD34+ HSC, HSP, and HSPC can be enriched using anti-CD34 antibodies directly or indirectly conjugated to magnetic particles in connection with a magnetic cell separator, for example, the CliniMACS® Cell Separation System (Miltenyi Biotec, Bergisch Gladbach, Germany).

### (iii) GENETICALLY MODIFYING CELL POPULATIONS TO EXPRESS CHIMERIC ANTIGEN RECEPTORS (CAR)

Cell populations are genetically modified to express chimeric antigen receptors (CAR) described herein.

### (iii-a) GENETIC ENGINEERING TECHNIQUES

Desired genes encoding CAR disclosed herein can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressible by the cell and, in certain instances, preferably heritable and expressible by its cell progeny.

The term “gene” refers to a nucleic acid sequence (used interchangeably with polynucleotide or nucleotide sequence) that encodes a CAR including a Nectin-4-binding domain as described herein. This definition includes various sequence polymorphisms, mutations, and/or sequence variants wherein such alterations do not substantially affect the function of the encoded CAR. The term “gene” may include not only coding sequences but also regulatory regions such as promoters, enhancers, and termination regions. Gene sequences encoding the molecule can be DNA or RNA that directs the expression of the CAR. These nucleic acid sequences may be a DNA strand sequence that is transcribed into RNA or an RNA sequence that is translated into protein. The sequences can also include degenerate codons of the native sequence or sequences that may be introduced to provide codon preference in a specific cell type (e.g., a mammalian cell). Portions of complete gene sequences are referenced throughout the disclosure as is understood by one of ordinary skill in the art.

Gene sequences encoding CAR are provided herein and can also be readily prepared by synthetic or recombinant methods from the relevant amino acid sequences and other description provided herein. In embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence.

“Encoding” refers to the property of specific sequences of nucleotides in a gene, such as a cDNA, or an mRNA, to serve as templates for synthesis of other macromolecules such as a defined sequence of amino acids.

Polynucleotide gene sequences encoding more than one portion of an expressed CAR can be operably linked to each other and relevant regulatory sequences. For example, there can be a functional linkage between a regulatory sequence and an exogenous nucleic acid sequence resulting in expression of the latter. For instance, a promoter is operably linked to a coding sequence if the promoter affects the transcription or expression of the coding sequence.

A “vector” is a nucleic acid molecule that is capable of transporting another nucleic acid. Vectors may be, e.g., plasmids (DNA plasmids or RNA plasmids), transposon-based systems, cosmids, bacterial artificial chromosomes, viruses, or phage. An “expression vector” is a vector that is capable of directing the expression of a protein encoded by one or more genes carried by the vector when it is present in the appropriate environment.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

A lentiviral vector is a vector derived from at least a portion of a lentivirus genome, including especially a self-inactivating lentiviral vector as provided in Milone et ah, Mol. Ther. 17(8): 1453-1464 (2009). Other examples of lentivirus vectors that may be used in the clinic, include: the LENTIVECTOR® gene delivery technology from Oxford BioMedica, the LENTIMAX™ vector system from Lentigen and the like.

There are a large number of available viral vectors suitable within the current disclosure, including those identified for human gene therapy applications (see Pfeifer and Verma, 2001, Ann. Rev. Genomics Hum. Genet. 2:177). Methods of using retroviral and lentiviral viral vectors and packaging cells for transducing mammalian host cells with viral particles including CAR transgenes are described in, e.g., U.S. Pat. No. 8,119,772; Walchli, et al., 2011, PLoS One 6:327930; Zhao, et al., 2005, J. Immunol. 174:4415; Engels, et al., 2003, Hum. Gene Ther. 14:1155; Frecha, et al., 2010, Mol. Ther. 18:1748; and Verhoeyen, et al., 2009, Methods Mol. Biol. 506:97. Retroviral and lentiviral vector constructs and expression systems are also commercially available.

Targeted genetic engineering approaches may also be utilized. The CRISPR (Clustered Regularly Interspaced Short Palindromic Repeats)/Cas (CRISPR-associated protein) nuclease system is an engineered nuclease system used for genetic engineering that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. For information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. For information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(I):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al. Nature biotechnology 29, 143-148 (2011); Christian, et al. Genetics 186, 757-761 (2010); Boch, et al. Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments can use transposon-based systems as gene editing agents to mediate the integration of a CAR construct into cells.

Several transposon/transposase systems have been adapted for genetic insertions of heterologous DNA sequences. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himar1, Passport, Minos, Ac/Ds, PIF, Harbinger, Harbinger3-DR, HSmar1, and spinON. Transposases and transposon systems are further described in U.S. Pat. Nos. 6,489,458; 7,148,203; 8,227,432; and 9,228,180.

Nanoparticles that result in selective in vivo genetic modification of targeted cell types can be used within the teachings of the current disclosure. In particular embodiments, the nanoparticles can be those described in WO2014153114, WO2017181110, and WO201822672.

### (iii-b) CAR SUBCOMPONENTS

As described previously, CAR include several distinct subcomponents that allow genetically modified cells to recognize and kill unwanted cells, such as cancer cells. The subcomponents include at least an extracellular component and an intracellular component. The extracellular component includes a binding domain that specifically binds a marker that is preferentially present on the surface of unwanted cells. When the binding domain binds such markers, the intracellular component activates the cell to destroy the bound cell. CAR additionally include a transmembrane domain that links the extracellular component to the intracellular component, and other subcomponents that can increase the CAR's function. For example, the inclusion of a spacer and/or one or more linker sequences can allow the CAR to have additional conformational flexibility, often increasing the binding domain's ability to bind the targeted cell marker.

### (iii-b-i) BINDING DOMAINS

The current disclosure provides binding domains for use in CAR that bind Nectin-4.

Antibodies are one example of binding domains and include whole antibodies or binding fragments of an antibody, e.g., Fv, Fab, Fab′, F(ab′)2, and single chain (sc) forms and fragments thereof that bind specifically a cellular marker (such as Nectin-4). Antibodies or antigen binding fragments can include all or a portion of polyclonal antibodies, monoclonal antibodies, human antibodies, humanized antibodies, synthetic antibodies, non-human antibodies, recombinant antibodies, chimeric antibodies, bispecific antibodies, mini bodies, and linear antibodies.

Antibodies are produced from two genes, a heavy chain gene and a light chain gene. Generally, an antibody includes two identical copies of a heavy chain, and two identical copies of a light chain. Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3). CDR regions are flanked by framework residues (FR).

The assignment of amino acids to each domain can be in accordance with Kabat numbering (Kabat et al. (1991), “Sequences of Proteins of Immunological Interest,” 5th Ed. Public Health Service, National Institutes of Health, Bethesda, Md. (“Kabat” numbering scheme)); Chothia (Al-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), IMGT (Lefranc M P et al., “IMGT unique numbering for immunoglobulin and T-cell receptor variable domains and Ig superfamily V-like domains,” Dev Comp Immunol, 2003 January; 27(1):55-77 (“IMGT” numbering scheme)), AHo (Honegger A and Plückthun A, “Yet another numbering scheme for immunoglobulin variable domains: an automatic modeling and analysis tool,” J Mol Biol, 2001 Jun. 8; 309(3):657-70, (AHo numbering scheme)), North (North et al., J Mol Biol. 406(2):228-256 (2011), “A new clustering of antibody CDR loop conformations”), or other numbering schemes. CDR residues can be identified using software programs such as ABodyBuilder. The boundaries of a given CDR or FR may vary depending on the scheme used for identification.

In some instances, scFvs based on the binding domains described herein and for use in a CAR can be prepared according to methods known in the art (see, for example, Bird et al., (1988) Science 242:423-426 and Huston et al., (1988) Proc. Natl. Acad. Sci. USA 85:5879-5883). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. scFv are commonly used as the binding domains of CAR.

In particular embodiments, the CAR includes a binding domain that binds Nectin-4. In particular embodiments, Nectin-4 includes the sequence as set forth in SEQ ID NO: 4.

In particular embodiments, the binding domain that binds Nectin-4 is an scFv. In particular embodiments, the binding domain that binds Nectin-4 is an scFV derived from the murine antibody M22-321b41.1. In particular embodiments, the binding domain that binds Nectin-4 is encoded by the sequence as set forth in SEQ ID NO: 54 or SEQ ID NO: 56. In particular embodiments, the binding domain that binds Nectin-4 is set forth in SEQ ID NO: 53 or SEQ ID NO: 55.

In particular embodiments, the M22-321b41.1 scFv includes the sequence:

(SEQ ID NO: 53)
DIVMTQAAFSNPVTLGTSASISCRSSKSLLHSNGITYLYWYLQKPGQSP
QLLIYHMSNLASGVPDRFTSSGSGTDFTLRISRVEAEDVGVYYCAQNLE
LPFTFGGGTKLETKRGGGGSGGGGSGGGGSQVQLQQSGAELARPGASVK
LSCKASGYTFTTYWMQWVKQRPGQGLEWIGSIYPGDGDTRYTQKFKGKA
TLTADKSSSTAYIQLSTLASEDSAVYYCAREYYGLDYWGQGTTLTVSS.

In particular embodiments, the M22-321b41.1 scFv is encoded by the sequence:

(SEQ ID NO: 54)
GATATTGTAATGACGCAGGCGGCATTTTCTAATCCGGTTACCCTCGGGA
CCTCAGCTTCTATCTCTTGTAGAAGTTCCAAATCCCTGCTTCATTCCAA
CGGCATTACTTACCTTTATTGGTACCTGCAAAAACCTGGGCAGTCACCA
CAACTCCTGATATATCACATGTCTAATTTGGCATCTGGGGTCCCCGATC
GGTTTACTTCATCAGGGTCCGGGACTGACTTCACGCTGCGCATCTCCAG
GGTTGAGGCAGAAGATGTTGGCGTCTACTACTGCGCTCAGAATTTGGAA
CTCCCGTTTACTTTCGGTGGAGGCACTAAGTTGGAAACAAAGCGAGGAG
GTGGCGGATCTGGTGGAGGAGGGAGTGGTGGGGGGGGTTCTCAAGTTCA
GTTGCAACAGTCCGGGGCAGAGCTGGCTCGACCAGGTGCCTCCGTAAAA
CTCTCTTGCAAAGCATCTGGATATACCTTTACAACTTACTGGATGCAAT
GGGTAAAACAGCGCCCCGGTCAGGGACTTGAGTGGATAGGCTCTATTTA
TCCAGGCGACGGAGATACTAGATACACACAGAAGTTCAAAGGGAAGGCG
ACGCTGACAGCTGATAAAAGCTCAAGCACAGCATACATTCAACTGTCCA
CTCTTGCGAGTGAAGACAGTGCCGTTTATTACTGTGCGCGAGAATACTA
CGGGCTGGATTACTGGGGCCAAGGTACGACCCTCACCGTGTCATCT.

In particular embodiments, the Nectin-4 scFv includes the sequence:

(SEQ ID NO: 55)
QVQLQQSGAELARPGASVKLSCKASGYTFTTYWMQWVKQRPGQGLEWIG
SIYPGDGDTRYTQKFKGKATLTADKSSSTAYIQLSTLASEDSAVYYCAR
EYYGLDYWGQGTTLTVSSGGGGSGGGGSGGGGSDIVMTQAAFSNPVTLG
TSASISCRSSKSLLHSNGITYLYWYLQKPGQSPQLLIYHMSNLASGVPD
RFTSSGSGTDFTLRISRVEAEDVGVYYCAQNLELPFTFGGGTKLETKR.

In particular embodiments, the Nectin-4 scFv is encoded by the sequence:

(SEQ ID NO: 56)
CAAGTTCAGTTGCAACAGTCCGGGGCAGAGCTGGCTCGACCAGGTGCCT
CCGTAAAACTCTCTTGCAAAGCATCTGGATATACCTTTACAACTTACTG
GATGCAATGGGTAAAACAGCGCCCCGGTCAGGGACTTGAGTGGATAGGC
TCTATTTATCCAGGCGACGGAGATACTAGATACACACAGAAGTTCAAAG
GGAAGGCGACGCTGACAGCTGATAAAAGCTCAAGCACAGCATACATTCA
ACTGTCCACTCTTGCGAGTGAAGACAGTGCCGTTTATTACTGTGCGCGA
GAATACTACGGGCTGGATTACTGGGGCCAAGGTACGACCCTCACCGTGT
CATCTGGAGGTGGCGGATCTGGTGGAGGAGGGAGTGGTGGGGGGGGTTC
TGATATTGTAATGACGCAGGCGGCATTTTCTAATCCGGTTACCCTCGGG
ACCTCAGCTTCTATCTCTTGTAGAAGTTCCAAATCCCTGCTTCATTCCA
ACGGCATTACTTACCTTTATTGGTACCTGCAAAAACCTGGGCAGTCACC
ACAACTCCTGATATATCACATGTCTAATTTGGCATCTGGGGTCCCCGAT
CGGTTTACTTCATCAGGGTCCGGGACTGACTTCACGCTGCGCATCTCCA
GGGTTGAGGCAGAAGATGTTGGCGTCTACTACTGCGCTCAGAATTTGGA
ACTCCCGTTTACTTTCGGTGGAGGCACTAAGTTGGAAACAAAGCGA.

In particular embodiments, the Nectin-4 binding domain includes a variable heavy chain including the sequence:

(SEQ ID NO: 57)
QVQLQQSGAELARPGASVKLSCKASGYTFTTYWMQWVKQRPGQGLEWIG
SIYPGDGDTRYTQKFKGKATLTADKSSSTAYIQLSTLASEDSAVYYCAR
EYYGLDYWGQGTTLTVSS.

In particular embodiments, the Nectin-4 binding domain includes a variable light chain including the sequence:

(SEQ ID NO: 58)
DIVMTQAAFSNPVTLGTSASISCRSSKSLLHSNGITYLYWYLQKPGQSP
QLLIYHMSNLASGVPDRFTSSGSGTDFTLRISRVEAEDVGVYYCAQNLE
LPFTFGGGTKLETKR.

In particular embodiments, the Nectin-4 binding domain includes a variable heavy chain encoded by the sequence:

(SEQ ID NO: 59)
CAAGTTCAGTTGCAACAGTCCGGGGCAGAGCTGGCTCGACCAGGTGCCT
CCGTAAAACTCTCTTGCAAAGCATCTGGATATACCTTTACAACTTACTG
GATGCAATGGGTAAAACAGCGCCCCGGTCAGGGACTTGAGTGGATAGGC
TCTATTTATCCAGGCGACGGAGATACTAGATACACACAGAAGTTCAAAG
GGAAGGCGACGCTGACAGCTGATAAAAGCTCAAGCACAGCATACATTCA
ACTGTCCACTCTTGCGAGTGAAGACAGTGCCGTTTATTACTGTGCGCGA
GAATACTACGGGCTGGATTACTGGGGCCAAGGTACGACCCTCACCGTGT
CATCT.

In particular embodiments, the Nectin-4 binding domain includes a variable light chain encoded by the sequence:

(SEQ ID NO: 60)
GATATTGTAATGACGCAGGCGGCATTTTCTAATCCGGTTACCCTCGGGA
CCTCAGCTTCTATCTCTTGTAGAAGTTCCAAATCCCTGCTTCATTCCAA
CGGCATTACTTACCTTTATTGGTACCTGCAAAAACCTGGGCAGTCACCA
CAACTCCTGATATATCACATGTCTAATTTGGCATCTGGGGTCCCCGATC
GGTTTACTTCATCAGGGTCCGGGACTGACTTCACGCTGCGCATCTCCAG
GGTTGAGGCAGAAGATGTTGGCGTCTACTACTGCGCTCAGAATTTGGAA
CTCCCGTTTACTTTCGGTGGAGGCACTAAGTTGGAAACAAAGCGA.

Referring to the Nectin-4 binding domains provided herein, the following CDR sets are provided. A CDR set refers to 3 light chain CDRs and 3 heavy chain CDRs that together result in binding to Nectin-4.

TABLE 1
Nectin-4-Binding CDR Sequences.

| CDR | | | SEQ |
|---|---|---|---|
| Definition | CDR | Sequence | ID NO: |
| North | CDRH1 | KASGYTFTTYWMQ | 61 |
| | CDRH2 | SIYPGDGDTR | 62 |
| | CDRH3 | AREYYGLDY | 63 |
| | CDRL1 | RSSKSLLHSNGITYLY | 64 |
| | CDRL2 | YHMSNLAS | 65 |
| | CDRL3 | AQNLELPFT | 66 |
| Kabat | CDRH1 | TYWMQ | 67 |
| | CDRH2 | SIYPGDGDTRYTQKFKG | 68 |
| | CDRH3 | EYYGLDY | 69 |
| | CDRL1 | RSSKSLLHSNGITYLY | 64 |
| | CDRL2 | HMSNLAS | 70 |
| | CDRL3 | AQNLELPFT | 66 |
| IMGT | CDRH1 | GYTFTTYW | 71 |
| | CDRH2 | IYPGDGDT | 72 |
| | CDRH3 | AREYYGLDY | 63 |
| | CDRL1 | KSLLHSNGITY | 73 |
| | CDRL2 | HMS | N/A |
| | CDRL3 | AQNLELPFT | 66 |
| Chothia | CDRH1 | GYTFTTY | 74 |
| | CDRH2 | YPGDGD | 75 |
| | CDRH3 | EYYGLDY | 69 |
| | CDRL1 | RSSKSLLHSNGITYLY | 64 |
| | CDRL2 | HMSNLAS | 70 |
| | CDRL3 | AQNLELPFT | 66 |
| Contact | CDRH1 | TTYWMQ | 76 |
| | CDRH2 | WIGSIYPGDGDTR | 77 |
| | CDRH3 | AREYYGLD | 78 |
| | CDRL1 | ITYLYWY | 79 |
| | CDRL2 | LLIYHMSNLA | 80 |
| | CDRL3 | AQNLELPF | 81 |

CDR predictions were generated using the program SAbPrep http://opig.stats.ox.ac.uk/webapps/newsabdab/sabpred/). ABodyBuilder within SAbPred was used (CDR predictions based on “Clothia”).

In particular embodiments, the binding domain includes a humanized antibody or an engineered fragment thereof. In particular embodiments, a non-human antibody is humanized, where one or more amino acid residues of the antibody are modified to increase similarity to an antibody naturally produced in a human or fragment thereof. These nonhuman amino acid residues are often referred to as “import” residues, which are typically taken from an “import” variable domain. As provided herein, humanized antibodies or antibody fragments include one or more CDRs from nonhuman immunoglobulin molecules and framework regions wherein the amino acid residues including the framework are derived completely or mostly from human germline. A humanized antibody can be produced using a variety of techniques known in the art, including CDR-grafting (see, e.g., European Patent No. EP 239,400; WO 91/09967; and U.S. Pat. Nos. 5,225,539, 5,530,101, and 5,585,089), veneering or resurfacing (see, e.g., EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., US 2005/0042664, US 2005/0048617, U.S. Pat. Nos. 6,407,213, 5,766,886, WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, Nectin-4 binding. These framework substitutions are identified by methods well-known in the art, e.g., by modeling of the interactions of the CDR and framework residues to identify framework residues important for Nectin-4 binding and sequence comparison to identify unusual framework residues at particular positions. (See, e.g., U.S. Pat. No. 5,585,089; and Riechmann et al., 1988, Nature, 332:323).

Although chimeric and humanized antibodies often incorporate all six CDRs from a non-human antibody, they can also be made with less than all CDRs (e.g., at least 3, 4, or 5) CDRs from a non-human antibody (e.g., Pascalis et al., J. Immunol. 169:3076, 2002; Vajdos et al., Journal of Molecular Biology, 320: 415-428, 2002; Iwahashi et al., Mol. Immunol.

Other binding fragments, such as Fv, Fab, Fab′, F(ab′)2, can also be used within the CAR disclosed herein. Additional examples of antibody-based binding domain formats for use in a CAR include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

In particular embodiments, a VL region in a binding domain of the present disclosure is derived from or based on a VL of an antibody disclosed herein and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the antibody disclosed herein. An insertion, deletion or substitution may be anywhere in the VL region, including at the amino- or carboxy-terminus or both ends of this region, provided that each CDR includes zero changes or at most one, two, or three changes and provided a binding domain containing the modified VL region can still specifically bind its target with an affinity similar to the wild type binding domain.

In particular embodiments, a binding domain VH region of the present disclosure can be derived from or based on a VH of an antibody disclosed herein and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of the antibody disclosed herein. An insertion, deletion or substitution may be anywhere in the VH region, including at the amino- or carboxy-terminus or both ends of this region, provided that each CDR includes zero changes or at most one, two, or three changes and provided a binding domain containing the modified VH region can still specifically bind its target with an affinity similar to the wild type binding domain.

In particular embodiments, a binding domain includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from an antibody disclosed herein or fragment or derivative thereof that specifically binds to Nectin-4.

### (iii-b-ii) SPACERS

Spacers are used to create appropriate distances and/or flexibility from other CAR sub-components. As indicated, in particular embodiments, the length of a spacer is customized for binding targeted (Nectin-4-expressing) cells and mediating destruction. In particular embodiments, a spacer length can be selected based upon the location of a cellular marker epitope, affinity of a binding domain for the epitope, and/or the ability of the Nectin-4-binding agent to mediate cell destruction following Nectin-4 binding.

Spacers typically include those having 10 to 250 amino acids, 10 to 200 amino acids, 10 to 150 amino acids, 10 to 100 amino acids, 10 to 50 amino acids, or 10 to 25 amino acids.

In particular embodiments, a spacer is 10 amino acids, 12 amino acids, 14 amino acids, 20 amino acids, 21 amino acids, 26 amino acids, 27 amino acids, 45 amino acids, 50 amino acids, 55 amino acids, 60 amino acids, 65 amino acids, 70 amino acids, or 75 amino acids. These lengths qualify as short spacers.

In particular embodiments, a spacer is 71 amino acids, 75 amino acids, 80 amino acids, 85 amino acids, 90 amino acids, 95 amino acids, 100 amino acids, 110 amino acids, 120 amino acids, 125 amino acids, 128 amino acids, 131 amino acids, 135 amino acids, 140 amino acids, 150 amino acids, 160 amino acids, or 179 amino acids. These lengths qualify as intermediate spacers.

In particular embodiments, a spacer is 180 amino acids, 190 amino acids, 200 amino acids, 210 amino acids, 212 amino acids, 214 amino acids, 216 amino acids, 218 amino acids, 220 amino acids, 228 amino acids, 230 amino acids, 240 amino acids, 250 amino acids, 260 amino acids, or 270 amino acids. These lengths qualify as long spacers.

In particular embodiments, spacers include all or a portion of an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region.

In particular embodiments, the spacer is a short spacer including an IgG4 hinge region. In particular embodiments, the spacer is an intermediate spacer including an IgG4 hinge region and an IgG4 CH3 region. In particular embodiments, the spacer is a long spacer including an IgG4 hinge region, an IgG4 CH2 region, and an IgG4 CH3 region. In particular embodiments, the long spacer includes a 4/2-N/Q mutation in the CH2 domain. Mutations can be used to prevent Fc-gamma receptor binding and activation-induced cell death.

In particular embodiments the IgG4 hinge region includes the sequence as set forth in SEQ ID NO: 9. In particular embodiments the IgG4 hinge region is encoded by any of SEQ ID NOs: 10, 11, or 12. In particular embodiments the IgG4 CH3 region includes the sequence as set forth in SEQ ID NOs: 17 or 18. In particular embodiments the IgG4 CH3 region is encoded by SEQ ID NOs: 19 or 20. In particular embodiments the IgG4 CH2 region includes the sequence as set forth in SEQ ID NOs: 13 or 14. In particular embodiments the IgG4 CH2 region is encoded by SEQ ID NOs: 15 or 16.

Other examples of hinge regions that can be used in CAR described herein include the hinge region present in the extracellular regions of type 1 membrane proteins, such as CD8α, CD4, CD28 and CD7, which may be wild-type or variants thereof.

In particular embodiments, a spacer includes a hinge region that includes a type II C-lectin interdomain (stalk) region or a cluster of differentiation (CD) molecule stalk region. A “stalk region” of a type II C-lectin or CD molecule refers to the portion of the extracellular domain (ECD) of the type II C-lectin or CD molecule that is located between the C-type lectin-like domain (CTLD; e.g., similar to CTLD of natural killer cell receptors) and the hydrophobic portion (transmembrane domain). For example, the ECD of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an ECD ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

### (iii-b-iii) TRANSMEMBRANE DOMAINS

As indicated, a transmembrane domain within a CAR serves to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 CD154, Toll-like receptor 1 (TLR1), TLR2, TLR3, TLR4, TLR5, TLR6, TLR7, TLR8, and TLR9. In particular embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDIId, ITGAE, CD103, ITGAL, CDIIa, ITGAM, CDIIb, ITGAX, CDIIc, ITGB1, CD29, ITGB2, CD18, ITGB7, TNFR2, DNAM1 (CD226), SLAMF4 (CD244, 2B4), CD84, CD96 (Tactile), CEACAM1, CRT AM, Ly9 (CD229), PSGL1, CD100 (SEMA4D), SLAMF6 (NTB-A, Lyl08), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge. In particular embodiments, the CAR includes a CD28 transmembrane domain. It has been shown that a CD28 transmembrane domain reduces the antigen-threshold for second-generation 4-1BB CAR T-cell activation.

In particular embodiments, a transmembrane domain has a three-dimensional structure that is thermodynamically stable in a cell membrane, and generally ranges in length from 15 to 30 amino acids. The structure of a transmembrane domain can include an α helix, a β barrel, a β sheet, a β helix, or any combination thereof.

A transmembrane domain can include one or more additional amino acids adjacent to the transmembrane region, e.g., one or more amino acid within the extracellular region of the CAR (e.g., up to 15 amino acids of the extracellular region) and/or one or more additional amino acids within the intracellular region of the CAR (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins to minimize interactions with other unintended members of the receptor complex. In particular embodiments, the transmembrane domain is encoded by the nucleic acid sequence encoding the CD28 transmembrane domain (SEQ ID NOs: 28, 29, 117, or 119). In particular embodiments, the transmembrane domain includes the amino acid sequence of the CD28 transmembrane domain (SEQ ID NOs: 25, 26, or 27).

### (iii-b-iv) INTRACELLULAR COMPONENTS

The intracellular component of a CAR includes an effector domains that is responsible for activation of the cell in which the CAR is expressed. The term “effector domain” is thus meant to include any portion of the intracellular domain sufficient to transduce an activation signal. An effector domain can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the intracellular effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T-cell receptor and a co-stimulatory domain which can include the cytoplasmic sequence from co-receptor or co-stimulatory molecule.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular embodiments, exemplary intracellular effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, NKp46, TLR1, TLR2, TLR3, TLR4, TLR5, TLR6, TLR7, TLR8, or TLR9. In particular embodiments, the effector domain includes a CD3ζ signaling domain. In some aspects it may be useful to have short-lived high amplitude signaling. Such short-lived high amplitude signaling such as in constructs including CD28 may allow for repeat dosing. Using intravesical installation, in some instances systemic persistence may not be needed.

Intracellular signaling domains that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (Fcε Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling domains include the cytoplasmic sequences of the CD3ζ chain, and/or co-receptors that act in concert to initiate signal transduction following binding domain engagement.

A co-stimulatory domain is a domain whose activation can be required for an efficient lymphocyte response to cellular marker binding. Some molecules are interchangeable as intracellular signaling domains or co-stimulatory domains. Examples of costimulatory domains include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART-cells in vitro and augments human T-cell persistence and anti-cancer activity in vivo (Song et al. Blood. 2012; 119(3):696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CDIId, ITGAE, CD103, ITGAL, CDIIa, ITGAM, CDIIb, ITGAX, CDIIc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, and CD19a. In particular embodiments, the co-stimulatory domain includes a 4-1BB signaling domain. In particular embodiments, the co-stimulatory domain includes a CD28 and 4-1BB signaling domain. In particular embodiments, the co-stimulatory domain includes a CD28 signaling domain.

In particular embodiments, the nucleic acid sequences encoding the intracellular component include a CD3ζ encoding sequence (SEQ ID NOs: 41 or 42) and a variant of the 4-1BB signaling encoding sequence (SEQ ID NOs: 35, 36, or 37). In particular embodiments, the amino acid sequence of the intracellular component includes a variant of CD3ζ (SEQ ID NOs: 38, 39, or 40) and a portion of the 4-1BB (SEQ ID NO: 32, 33, or 34) intracellular component. In particular embodiments, the nucleic acid sequences encoding the intracellular component include a CD3ζ encoding sequence (SEQ ID NOs: 41 or 42) and a variant of the CD28 signaling domain encoding sequence (SEQ ID NO: 116). In particular embodiments, the amino acid sequence of the intracellular component includes a variant of CD3ζ (SEQ ID NOs: 38, 39, or 40) and a portion of the CD28 intracellular component (SEQ ID NO. 115).

In particular embodiments, the intracellular component includes (i) all or a portion of the signaling domain of CD3ζ, (ii) all or a portion of the signaling domain of 4-1BB, (iii) all or a portion of the signaling domain of CD28 signaling domain, (iv) all or a portion of the signaling domain of CD3ζ and 4-1BB, (v) all or a portion of the signaling domain of CD3ζ and CD28, or (vi) or all or a portion of the signaling domain of CD3ζ, 4-1BB, and CD28.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

### (iii-b-v) LINKERS

As used herein, a linker can include a chemical moiety that serves to connect two other subcomponents of the molecule. Some linkers serve no purpose other than to link components while many linkers serve an additional purpose. Linkers can, for example, link VL and VH of antibody derived binding domains of scFvs and serve as junction amino acids between subcomponent portions of a CAR.

Linkers can be flexible, rigid, or semi-rigid, depending on the desired function of the linker. Linkers can include junction amino acids. For example, in particular embodiments, linkers provide flexibility and room for conformational movement between different components of CAR. Commonly used flexible linkers include Gly-Ser linkers. In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 82), (Gly₃Ser)ₙ(Gly₄Ser)ₙ (SEQ ID NO: 83), (Gly₃Ser)ₙ(Gly₂Ser)ₙ (SEQ ID NO: 84), or (Gly₃Ser)ₙ(Gly₄Ser)₁ (SEQ ID NO: 85). In particular embodiments, the linker is (Gly₄Ser)₄ (SEQ ID NO: 86), (Gly₄Ser)₃ (SEQ ID NO: 87), (Gly₄Ser)₂ (SEQ ID NO: 88), (Gly₄Ser)₁ (SEQ ID NO: 89), (Gly₃Ser)₂ (SEQ ID NO: 90), (Gly₃Ser)₁ (SEQ ID NO: 91), (Gly₂Ser)₂ (SEQ ID NO: 92) or (Gly₂Ser)₁, GGSGGGSGGSG (SEQ ID NO: 93), GGSGGGSGSG (SEQ ID NO: 94), or GGSGGGSG (SEQ ID NO: 95).

In particular embodiments, a linker region is (GGGGS)ₙ (SEQ ID NO: 96) wherein n is an integer including, 1, 2, 3, 4, 5, 6, 7, 8, 9, or more. In particular embodiments, the linker is (EAAAK)ₙ (SEQ ID NO: 97) wherein n is an integer including 1, 2, 3, 4, 5, 6, 7, 8, 9, or more.

In some situations, flexible linkers may be incapable of maintaining a distance or positioning of CAR needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or noncleavable linker). In some aspects, the linker is a procharged linker, a hydrophilic linker, or a dicarboxylic acid-based linker.

Junction amino acids can be a linker which can be used to connect sequences when the distance provided by a spacer is not needed and/or wanted. For example, junction amino acids can be short amino acid sequences that can be used to connect co-stimulatory intracellular signaling components. In particular embodiments, junction amino acids are 9 amino acids or less (e.g., 2, 3, 4, 5, 6, 7, 8, or 9 amino acids). In particular embodiments, a glycine-serine doublet can be used as a suitable junction amino acid linker. In particular embodiments, a single amino acid, e.g., an alanine, a glycine, can be used as a suitable junction amino acid.

### (iii-b-vi) CONTROL FEATURES INCLUDING TAG CASSETTES, TRANSDUCTION MARKERS, AND/OR SUICIDE SWITCHES

In particular embodiments, CAR constructs can include one or more tag cassettes and/or transduction markers. Tag cassettes and transduction markers can be used to activate, promote proliferation of, detect, enrich for, isolate, track, deplete and/or eliminate genetically modified cells in vitro, in vivo and/or ex vivo. “Tag cassette” refers to a unique synthetic peptide sequence affixed to, fused to, or that is part of a CAR, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein. Transduction markers can serve the same purposes but are derived from naturally occurring molecules and are often expressed using a skipping element that separates the transduction marker from the rest of the CAR.

Tag cassettes that bind cognate binding molecules include, for example, His tag (HHHHHH; SEQ ID NO: 98), Flag tag (DYKDDDDK; SEQ ID NO: 99), Xpress tag (DLYDDDDK; SEQ ID NO: 100), Avi tag (GLNDIFEAQKIEWHE; SEQ ID NO: 101), Calmodulin tag (KRRWKKNFIAVSAANRFKKISSSGAL; SEQ ID NO: 102), Polyglutamate tag (EEEEEE; SEQ ID NO: 123), HA tag (YPYDVPDYA; SEQ ID NO: 103), Myc tag (EQKLISEEDL; SEQ ID NO: 104), Strep tag (which refers the original STREP® tag (WRHPQFGG; SEQ ID NO: 105), STREP® tag II (WSHPQFEK SEQ ID NO: 106 (IBA Institut fur Bioanalytik, Germany); see, e.g., U.S. Pat. No. 7,981,632), Softag 1 (SLAELLNAGLGGS; SEQ ID NO: 107), Softag 3 (TQDPSRVG; SEQ ID NO: 108), and V5 tag (GKPIPNPLLGLDST; SEQ ID NO: 109).

Conjugate binding molecules that specifically bind tag cassette sequences disclosed herein are commercially available. For example, His tag antibodies are commercially available from suppliers including Life Technologies, Pierce Antibodies, and GenScript.Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

Transduction markers may be selected from at least one of a truncated CD19 (tCD19; see Budde et al., Blood 122: 1660, 2013); a truncated human EGFR (tEGFR or EGFRt; see Wang et al., Blood 118: 1255, 2011); an ECD of human CD34; and/or RQR8 which combines target epitopes from CD34 (see Fehse et al, Mol. Therapy 1(5 Pt 1); 448-456, 2000) and CD20 antigens (see Philip et al, Blood 124: 1277-1278). In particular embodiments, cells are genetically modified to express EGFRt.

In particular embodiments, CAR constructs can include a polynucleotide that encodes a self-cleaving polypeptide, wherein the polynucleotide encoding the self-cleaving polypeptide is located between the polynucleotide encoding the CAR construct and a polynucleotide encoding a transduction marker (e.g., EGFRt).

Exemplary self-cleaving polypeptides include 2A peptide from porcine teschovirus-1 (P2A), Thosea asigna virus (T2A), equine rhinitis A virus (E2A), foot-and-mouth disease virus (F2A), or variants thereof. Further exemplary nucleic acid and amino acid sequences of 2A peptides are set forth in, for example, Kim et al. (PLOS One 6:e18556 (2011). In particular embodiments, cells are genetically modified to include a self-cleaving polypeptide. In particular embodiments, the self-cleaving polypeptide includes T2A.

Control features may be present in multiple copies in a CAR or can be expressed as distinct molecules with the use of a skipping element. For example, a CAR can have one, two, three, four or five tag cassettes and/or one, two, three, four, or five transduction markers could also be expressed. For example, embodiments can include a CAR construct having two Myc tag cassettes, or a His tag and an HA tag cassette, or a HA tag and a Softag 1 tag cassette, or a Myc tag and a SBP tag cassette. Exemplary transduction markers and cognate pairs are described in U.S. Ser. No. 13/463,247.

One advantage of including at least one control feature in a CAR is that cells expressing CAR administered to a subject can be increased or depleted using the cognate binding molecule to a tag cassette. In certain embodiments, the present disclosure provides a method for depleting a modified cell expressing a CAR by using an antibody specific for the tag cassette, using a cognate binding molecule specific for the control feature, or by using a second modified cell expressing a CAR and having specificity for the control feature. Elimination of modified cells may be accomplished using depletion agents specific for a control feature. For example, if EGFRt is used, then an anti-EGFRt binding domain (e.g., antibody, scFv) fused to or conjugated to a cell-toxic reagent (such as a toxin, radiometal) may be used, or an anti-EGFRt/anti-CD3 bispecific scFv, or an anti-EGFRt CAR T-cell may be used.

In particular embodiments, a polynucleotide encoding an iCaspase9 construct (iCasp9) may be inserted into a CAR construct as a suicide switch.

In certain embodiments, modified cells expressing a CAR may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu, et al., Theranostics 2:3, 2012).

Thus, modified cells expressing at least one control feature with a CAR can be, e.g., more readily identified, isolated, sorted, induced to proliferate, tracked, and/or eliminated as compared to a modified cell without a tag cassette.

### (iii-b-vii) MULTIMERIZATION DOMAINS

In particular embodiments, the CAR can optionally include a multimerization domain. A “multimerization domain” is a domain that causes two or more proteins (monomers) to interact with each other through covalent and/or non-covalent association(s). Multimerization domains present in proteins can result in protein interactions that form dimers, trimers, tetramers, pentamers, hexamers, heptamers, etc., depending on the number of units/monomers incorporated into the multimer.

In particular embodiments, the multimerization domain is a dimerization domain that allows binding of two complementary monomers to form a dimer. In particular embodiments, a dimerization and docking domain (DDD) can be derived from the cAMP-dependent protein kinase (PKA) regulatory subunits and can be paired with an anchoring domain (AD). The AD can be derived from a specific region found in various A-kinase anchoring proteins (AKAPs) that mediates association with the R subunits of PKA. Additional DDDs and ADs include: the 4-helix bundle type DDD (Newlon, et al. EMBO J. 2001; 20: 1651-1662; Newlon, et al. Nature Struct Biol. 1999; 3: 222-227) domains obtained from p53, DCoH (pterin 4 α carbinolamine dehydratase/dimerization cofactor of hepatocyte nuclear factor 1 α (TCF1)) and HNF-1 (hepatocyte nuclear factor 1) (Rose, et al. Nature Struct Biol. 2000; 7: 744-748). Other AD sequences of potential use may be found in US 2003/0232420A1.

In particular embodiments, complementary binding domains can dimerize. In particular embodiments, the binding domain is a transmembrane polypeptide derived from a FcεRI chain. In particular embodiments, a CAR can include a part of a FcεRI α chain and another CAR can include a part of an FcεRI β chain such that said FcεRI chains spontaneously dimerize together to form a dimeric CAR. In particular embodiments, CAR can include a part of a FcεRI α chain and a part of a FcεRI γ chain such that said FcεRI chains spontaneously trimerize together to form a trimeric CAR, and in another embodiment the multi-chain CAR can include a part of FcεRI α chain, a part of FcεRI β chain and a part of FcεRI γ chain such that said FcεRI chains spontaneously tetramerize together to form a tetrameric CAR.

Leucine zippers are described in U.S. Pat. No. 5,932,448; SH2 and SH3 are described in Vidal et al., Biochemistry, 43:7336-44, 2004); PTB is described in Zhou et al., Nature, 378:584-592, 1995); WW is described in Sudol Prog Biochys MoL Bio, 65:113-132, 1996; PDZ is described in Kim et al., Nature, 378: 85-88, 1995 and Komau et al., Science, 269:1737-1740, 1995; and WD40 is described in Hu et al., J Biol Chem., 273:33489-33494, 1998.

Additional multimerization domains and systems are described in, for example, Hodneland, et al. Proc Natl Acad Sci USA. 2002; 99: 5048-5052; Arakawa et al., J Biol. Chem., 269:27833-27839, 1994; Radziejewski et al., Biochem, 32: 1350, 1993; WO2012001647A2; U.S. Pat. No. 5,821,333; GenBank Accession no. AAF73912.1 (Nishi et al., Mol Cell Biol, 25: 2607-2621, 2005), the SH3 domain of IB1 from GenBank Accession no. AAD22543.1 (Kristensen el al., EMBO J., 25: 785-797, 2006), the PTB domain of human DOK-7 from GenBank Accession no. NP_005535.1 (Wagner et al., Cold Spring Harb Perspect Biol. 5: a008987, 2013), the PDZ-like domain of SATB1 from UniProt Accession No. Q01826 (Galande et al., Mol Cell Biol. August; 21: 5591-5604, 2001), the WD40 repeats of APAF from UniProt Accession No. 014727 (Jorgensen et al., 2009. PLOS One. 4(12):e8463), the PAS motif of the dioxin receptor from UniProt Accession No. I6L9E7 (Pongratz et al., Mol Cell Biol, 18:4079-4088, 1998) and the EF hand motif of parvalbumin from UniProt Accession No. P20472 (Jamalian et al., Int J Proteomics, 2014: 153712, 2014). C4b, dextrameric, and ferritin-based multimerization can be used.

In particular embodiments, complementary binding domains can be induced using a third molecule or chemical inducer. This method of dimerization requires that one CAR include a chemical inducer of dimerization binding domain 1 (CBD1) and the second CAR include the second chemical inducer of dimerization binding domain (CBD2), wherein CBD1 and CBD2 are capable of simultaneously binding to a chemical inducer of dimerization (CID). CBD1 may include a rapamycin binding domain of FK-binding protein 12 (FKBP12) and CBD2 may include a FKBP12-Rapamycin Binding (FRB) domain of mTOR.

### (iv) CHARACTERIZATION OF GENETICALLY ENGINEERED CELLS

In particular embodiments, the engineered cells can be assessed for surface expression of the CAR. In particular embodiments, at least 50%, at least 55%, at least 60%, at least 65%, at least 70%, at least 75%, at least 80%, at least 85%, at least 90%, at least 95%, or at least 99% of the engineered cells express a detectable level of the CAR.

Surface protein expression can be determined by flow cytometry or other appropriate methods well known to those of ordinary skill in the art.

### (v) CELL ACTIVATING CULTURE CONDITIONS

Cell populations can be incubated in a culture-initiating composition to expand cell populations. The incubation can be carried out in a culture vessel, such as a bag, cell culture plate, flask, chamber, chromatography column, cross-linked gel, cross-linked polymer, column, culture dish, hollow fiber, microtiter plate, silica-coated glass plate, tube, tubing set, well, vial, or other container for culture or cultivating cells.

In particular embodiments, the cell population can be incubated in the culture-initiating composition before or after genetic engineering the cell populations. In particular embodiments, the incubation can be carried out for 1 day to 6 days, 1 day to 5 days, 1 day to 4 days, 1 day to 3 days, 1 day to 2 days, or 1 day before genetically engineering the cell populations. In particular embodiments, the incubation can be carried out for 1 day to 6 days, 1 day to 5 days, 1 day to 4 days, 1 day to 3 days, 1 day to 2 days, or 1 day after genetically engineering the cell populations. In particular embodiments, the incubation can be carried out at the same time as genetically engineering the cell populations.

Culture conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177, Klebanoff et al. (2012) J Immunother. 35(9): 651-660, Terakura et al. (2012) Blood. 1:72-82, and/or Wang et al. (2012) J Immunother. 35(9):689-701.

Exemplary culture media for culturing T-cells include (i) RPMI supplemented with non-essential amino acids, sodium pyruvate, and penicillin/streptomycin; (ii) RPMI with HEPES, 5-15% human serum, 1-3% L-Glutamine, 0.5-1.5% penicillin/streptomycin, and 0.25×10-4-0.75×10-4 M β-MercaptoEthanol; (iii) RPMI-1640 supplemented with 10% fetal bovine serum (FBS), 2 mM L-glutamine, 10 mM HEPES, 100 U/ml penicillin and 100 m/mL streptomycin; (iv) DMEM medium supplemented with 10% FBS, 2 mM L-glutamine, 10 mM HEPES, 100 U/ml penicillin and 100 m/mL streptomycin; and (v) X-Vivo 15 medium (Lonza, Walkersville, MD) supplemented with 5% human AB serum (Gemcell, West Sacramento, CA), 1% HEPES (Gibco, Grand Island, NY), 1% Pen-Strep (Gibco), 1% GlutaMax (Gibco), and 2% N-acetyl cysteine (Sigma-Aldrich, St. Louis, MO). T-cell culture media are also commercially available from Hyclone (Logan, UT). Additional T-cell activating components that can be added to such culture media are described in more detail below.

In some embodiments, the T-cells are expanded by adding to the culture-initiating composition feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture (e.g., for a time sufficient to expand the numbers of T-cells). In some aspects, the non-dividing feeder cells can include gamma-irradiated PBMC feeder cells. In some embodiments, the PBMC are irradiated with gamma rays in the range of 3000 to 3600 rads to prevent cell division. In some aspects, the feeder cells are added to culture medium prior to the addition of the populations of T-cells.

Optionally, the incubation may further include adding non-dividing EBV-transformed lymphoblastoid cells (LCL) as feeder cells. LCL can be irradiated with gamma rays in the range of 6000 to 10,000 rads. The LCL feeder cells in some aspects is provided in any suitable amount, such as a ratio of LCL feeder cells to initial T lymphocytes of at least 10:1.

In some embodiments, the stimulating conditions include temperature suitable for the growth of human T lymphocytes, for example, at least 25° C., at least 30° C., or 37° C.

The activating culture conditions for T-cells include conditions whereby T-cells of the culture-initiating composition proliferate or expand. T-cell activating conditions can include one or more cytokines, for example, interleukin (IL)-2, IL-7, IL-15 and/or IL-21. IL-2 can be included at a range of 10-100 ng/ml (e.g., 40, 50, or 60 ng/ml). IL-7, IL-15, and/or IL-21 can be individually included at a range of 0.1-50 ng/ml (e.g., 5, 10, or 15 ng/ml). Particular embodiments utilize IL-2 at 50 ng/ml. Particular embodiments utilize, IL-7, IL-15 and IL-21 individually included at 10 ng/ml.

In particular embodiments, T-cell activating culture condition conditions can include T-cell stimulating epitopes. T-cell stimulating epitopes include CD3, CD27, CD2, CD4, CD5, CD7, CD8, CD28, CD30, CD40, CD56, CD83, CD90, CD95, 4-1BB (CD137), B7-H3, CTLA-4, Frizzled-1 (FZD1), FZD2, FZD3, FZD4, FZD5, FZD6, FZD7, FZD8, FZD9, FZD10, HVEM, ICOS, IL-1R, LAT, LFA-1, LIGHT, MHCI, MHCII, NKG2D, OX40, ROR2 and RTK.

In particular embodiments, a T-cell activating culture media includes a FACS-sorted T-cell population cultured within RPMI with HEPES, 5-15% human serum, 1-3% L-Glutamine, 0.5-1.5% Pen/strep, 0.25×10⁻⁴-0.75×10⁻⁴ M β-MercaptoEthanol, with IL-7, IL-15 and IL-21 individually included at 5-15 (e.g., 10) ng/ml. The culture is carried out on a flat-bottom well plate with 0.1-0.5×10⁶ plated cells/well. On Day 3 post activation cells are transferred to a tissue culture (TC)-treated plate.

In particular embodiments, a T-cell activating culture media includes a FACS-sorted CD8+ T population cultured within RPMI with HEPES, 10% human serum, 2% L-Glutamine, 1% Pen/strep, 0.5×10⁻⁴ M β-MercaptoEthanol, with IL-7, IL-15 and IL-21 individually included at 5-15 (e.g., 10) ng/ml. The culture is carried out on a flat-bottom non-tissue culture-treated 96/48-well plate with 0.1-0.5×10⁶ plated cells/well. On Day 3 post activation cells are transferred to TC-treated plate.

Culture conditions for HSC/HSP can include expansion with a Notch agonist (see, e.g., U.S. Pat. Nos. 7,399,633; 5,780,300; 5,648,464; 5,849,869; and 5,856,441 and growth factors present in the culture condition as follows: 25-300 ng/ml SCF, 25-300 ng/ml Flt-3L, 25-100 ng/ml TPO, 25-100 ng/ml IL-6 and 10 ng/ml IL-3. In more specific embodiments, 50, 100, or 200 ng/ml SCF; 50, 100, or 200 ng/ml of Flt-3L; 50 or 100 ng/ml TPO; 50 or 100 ng/ml IL-6; and 10 ng/ml IL-3 can be used.

### (vi) EX VIVO MANUFACTURED CELL FORMULATIONS

In particular embodiments, genetically modified cells can be harvested from a culture medium and washed and concentrated into a carrier in a therapeutically-effective amount. Herein, cell formulations refers to the formulations including cells genetically modified to express a CAR disclosed herein and prepared for administration. Exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), and combinations thereof.

In particular embodiments, carriers can be supplemented with human serum albumin (HSA) or other human serum components or fetal bovine serum. In particular embodiments, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, cell formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within cell formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In cell formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

In particular embodiments, cell formulations can include one or more genetically modified cell types (e.g., modified T-cells, NK cells, or stem cells). Cell formulations can include different types of genetically-modified cells (e.g., T-cells, NK cells, and/or stem cells in combination).

Different types of genetically-modified cells or cell subsets (e.g., modified T-cells, NK cells, and/or stem cells) can be provided in different ratios e.g., a 1:1:1 ratio, 2:1:1 ratio, 1:2:1 ratio, 1:1:2 ratio, 5:1:1 ratio, 1:5:1 ratio, 1:1:5 ratio, 10:1:1 ratio, 1:10:1 ratio, 1:1:10 ratio, 2:2:1 ratio, 1:2:2 ratio, 2:1:2 ratio, 5:5:1 ratio, 1:5:5 ratio, 5:1:5 ratio, 10:10:1 ratio, 1:10:10 ratio, 10:1:10 ratio, etc. These ratios can also apply to numbers of cells expressing the same or different CAR components. Particular embodiments include a 1:1 ratio of CD4 T-cells and CD8 T-cells.

The cell formulations disclosed herein can be prepared for administration by, e.g., injection, infusion, perfusion, or lavage. The cell formulations can further be formulated for bone marrow, intravenous, intradermal, intraarterial, intranodal, intralymphatic, intraperitoneal, intralesional, intraprostatic, intravaginal, intrarectal, intrathecal, intratumoral, intramuscular, intravesical, and/or subcutaneous injection.

### (vii) TARGETED VIRAL VECTORS & NANOPARTICLES FOR IN VIVO CELL MODIFICATION

Targeted viral vectors and/or nanoparticles can also be used to genetically-modify immune cells in vivo or ex vivo. Modifying formulations refers to the targeted viral vectors and/or nanoparticles used to genetically modify immune cells to express a CAR disclosed herein within a pharmaceutically acceptable carrier. Viral vectors that can be used to deliver CAR-encoding genes to cells are described elsewhere herein, and numerous targeted (e.g., pseudotyped) viral vectors are known in the art.

Exemplary cell-targeted nanoparticles include a cell targeting ligand (e.g., that binds CD3, CD4, CD8, CD34) on the surface of the nanoparticle wherein the cell targeting ligand results in selective uptake of the nanoparticle by a selected cell type. The nanoparticle then delivers gene modifying components that result in expression of the CAR.

Exemplary nanoparticles include liposomes (microscopic vesicles including at least one concentric lipid bilayer surrounding an aqueous core), liposomal nanoparticles (a liposome structure used to encapsulate another smaller nanoparticle within its core); and lipid nanoparticles (liposome-like structures that lack the continuous lipid bilayer characteristic of liposomes). Other polymer-based nanoparticles can also be used as well as porous nanoparticles constructed from any material capable of forming a porous network. Exemplary materials include metals, transition metals and metalloids (e.g., lithium, magnesium, zinc, aluminum and silica).

For in vivo delivery and cellular uptake, nanoparticles can have a neutral or negatively-charged coating and a size of 130 nm or less. Dimensions of the nanoparticles can be determined using, e.g., conventional techniques, such as dynamic light scattering and/or electron microscopy. In particular embodiments, the nanoparticles can be those described in WO2014153114, WO2017181110, and WO201822672.

Therapeutically effective amounts of vectors and/or nanoparticles within modifying formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

### (viii) METHODS OF USE

Methods disclosed herein include treating subjects (humans, non-human primates, veterinary animals (dogs, cats, reptiles, birds, etc.) livestock (horses, cattle, goats, pigs, chickens, etc.) and research animals (monkeys, rats, mice, fish, etc.)) with formulations disclosed herein (cell formulations and/or modifying formulations). Treating subjects includes delivering therapeutically effective amounts. Therapeutically effective amounts include those that provide effective amounts, prophylactic treatments and/or therapeutic treatments.

An “effective amount” is the amount of a formulation necessary to result in a desired physiological change in the subject. For example, an effective amount can provide an immunogenic anti-cancer effect. Effective amounts are often administered for research purposes. Effective amounts disclosed herein can cause a statistically significant effect in an animal model or in vitro assay relevant to the assessment of a cancer's development or progression. An immunogenic formulation can be provided in an effective amount, wherein the effective amount stimulates an immune response.

A “prophylactic treatment” includes a treatment administered to a subject who does not display signs or symptoms of a cancer or displays only early signs or symptoms of a cancer such that treatment is administered for the purpose of diminishing or decreasing the risk of developing the cancer further. Thus, a prophylactic treatment functions as a preventative treatment against a Nectin-4-expressing cancer. In particular embodiments, prophylactic treatments reduce, delay, or prevent metastasis from a primary a cancer tumor site from occurring.

A “therapeutic treatment” includes a treatment administered to a subject who displays symptoms or signs of a cancer and is administered to the subject for the purpose of diminishing or eliminating those signs or symptoms of the cancer. The therapeutic treatment can reduce, control, or eliminate the presence or activity of the cancer and/or reduce control or eliminate side effects of the cancer.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo- or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

In particular embodiments, therapeutically effective amounts induce an immune response. The immune response can be against a Nectin-4-expressing cancer cell.

“Nectin-4-positive cancer cell” refers to a cancer cell that expresses Nectin-4 on its surface. In some embodiments, expression of Nectin-4 on the cell surface is determined, for example, using antibodies to Nectin-4 in a method such as immunohistochemistry, FACS, etc. Alternatively, Nectin-4 mRNA expression is considered to correlate to Nectin-4 expression on the cell surface and can be determined by, for example, in situ hybridization and/or RT-PCR (including quantitative RT-PCR).

Examples of Nectin-4-related disorders that can be treated with CAR disclosed herein include bladder cancer, breast cancer, ovarian cancer, esophageal cancer, lung cancer, colorectal cancer, head and neck squamous cell cancer, and pancreatic cancer. In particular embodiments, bladder cancer includes urothelial carcinoma, squamous cell carcinoma, and adenocarcinoma. In particular embodiments, breast cancer includes triple-negative breast cancer (TNBC) and basal breast cancer.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer, stage of, previous or concurrent therapeutic interventions, idiopathy of the subject and route of administration.

Therapeutically effective amounts of cell formulations can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts of modifying formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intravaginal, intrarectal, topical, intrathecal, intratumoral, intramuscular, intravesical, and/or subcutaneous administration. In particular embodiments, cell formulations and/or modifying formulations are administered by intravesically.

In particular embodiments, a method of treating bladder (e.g., urothelial) cancer within a subject includes intravesical administration. Intravesical administration includes the administration of medication directly into the bladder (e.g., via catheterization). Prior to intravesical administration, an assessment of the subject's ability to retain solution for a select dwell time can be performed. In particular embodiments, the subject's bladder should be emptied before instillation of the formulation. In particular embodiments, a patient experiencing bladder spasms can be administered an anticholinergic.

Intravesical instillation of a formulation disclosed herein will establish a depot of drug within the bladder, providing sustained release of formulation within the bladder over time. Intravesical instillation procedures are known in the art. For intravesical instillation, the formulation should be at suitable volume to supply a sufficient dose volume for intravesical instillation, i.e., where the volume of the dose is sufficient to expose the bladder tissues to the formulation. Generally, the volume of the formulation (including carrier and any diluent) is less than 100 mL. In some embodiments, the volume of the formulation is 10 mL to 100 mL, or 20 mL to 80 mL, or 25 mL to 75 mL, or 10 mL to 50 mL, or 15 mL to 45 mL, or 20 mL to 40 mL, or 25 mL to 35 mL, or 20 mL to 30 mL, or 25 mL.

In particular embodiments, dwell time includes 15 minutes, 30 minutes, 1.0 hour, 1.1 hours, 1.2 hours, 1.3 hours, 1.4 hours, 1.5 hours, 1.6 hours, 1.7 hours, 1.8 hours, 1.9 hours, 2.0 hours, 2.1 hours, 2.2 hours, 2.3 hours, 2.4 hours, 2.5 hours, 2.6 hours, 2.7 hours, 2.8 hours, 2.9 hours, 3.0 hours, 3.1 hours, 3.2 hours, 3.3 hours, 3.4 hours, 3.5 hours, 3.6 hours, 3.7 hours, 3.8 hours, 3.9 hours, or 4.0 hours. In particular embodiments, dwell time includes 30 minutes to 2 hours.

One or more subsequent instillations of the formulation can be administered following the initial instillation. The subsequent instillations can be separated by periodic intervals. In particular embodiments, periodic intervals includes a day, two days, three days, four days, five days, six days, seven days, two weeks, three weeks, a month, two months, or three months.

In particular embodiments, 5 instillations can be administered on a weekly basis two weeks following the initial instillation, and additionally, 3 more instillations can be administered weekly 3 months after the 5th instillation, followed by 3 more instillations administered weekly 3 months after the 8th instillation, followed by 3 more instillations administered weekly 3 months after the 11th instillation, for a total of 14 instillations administered after the initial instillation. Devices such as catheters and needles can be used to administer the formulation to the bladder.

In particular embodiments, intravesical administration includes injecting formulation into one or more tumor surgical resection sites, wherein the injecting is done following surgical resection of one or more tumors of the subject, thereby treating or inhibiting the recurrence of the cancer.

In certain embodiments, cell formulations and/or modifying formulations are administered to a patient in conjunction with (e.g., before, simultaneously or following) any number of relevant treatment modalities. In particular embodiments, cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycolipenic acid, steroids, FR901228, cytokines, and irradiation.

In certain embodiments, cell formulations and/or modifying formulations (or formulations) may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-II; topoisomerase inhibitor RFS2000; difluoromethylomithine (DMFO); retinoic acid derivatives such as Targretin™ (bexarotene), Panretin™, (abtretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprobde, and goserebn; and pharmaceutically acceptable salts, acids or derivatives of any of the above. Combinations of chemotherapeutic agents are also administered where appropriate, including, CHOP, i.e., Cyclophosphamide (Cytoxan®), Doxorubicin (hydroxydoxorubicin), Vincristine (Oncovin®), and Prednisone.

In some embodiments, the chemotherapeutic agent is administered at the same time or within one week after the administration of the cell formulation and/or modifying formulation. In other embodiments, the chemotherapeutic agent is administered from 1 to 4 weeks or from 1 week to 1 month, 1 week to 2 months, 1 week to 3 months, 1 week to 6 months, 1 week to 9 months, or 1 week to 12 months after the administration of the cell formulation and/or modifying formulation. In other embodiments, the chemotherapeutic agent is administered at least 1 month before administering the cell formulation and/or modifying formulation. In some embodiments, the methods further include administering two or more chemotherapeutic agents.

In additional embodiments, formulations can be administered with an anti-inflammatory agent. Anti-inflammatory agents or drugs include steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of propoxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofm) and intramuscular) and minocycline.

In certain embodiments, formulations described herein are administered in conjunction with a cytokine. “Cytokine” as used herein is meant to refer to proteins released by one cell population that act on another cell as intercellular mediators. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-I, IL-1 alpha, IL-2, IL-3, IL-4, IL-5, IL-6, IL-7, IL-8, IL-9, IL-10, IL-II, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### (ix) KITS

The current disclosure also includes kits. Kits can include various components to practice methods disclosed herein. For example, depending on the aspect of the methods practiced, kits could include one or more of nucleic acids encoding a CAR disclosed herein; a protein encoding sequence as set forth in SEQ ID NO: 3 or SEQ ID NO: 1; vector (e.g., lentiviral vector) carrying Nectin-4 CAR construct with short, intermediate, and/or long spacers; a nucleic acid encoding an scFv; a nucleic acid encoding a VL; a nucleic acid encoding a VH; a nucleic acid encoding a transmembrane domain; a nucleic acid encoding EGFRt; cells (e.g., immune cells, T-cells, CD4 T-cells, CD8 T-cells, B cells, natural killer (NK) cells, NK-T-cells, monocytes/macrophages, lymphocytes, hematopoietic stem cells (HSCs), hematopoietic progenitor cells (HPC), and/or a mixture of HSC and HPC (i.e., HSPC), untransduced T-cells, Nectin-4 CAR T-cells); cell lines (e.g., UM-UC-3, SCABER, SW780, HT1376, UM-UC-9, CoCaB1, CoCaB11, or UM-UC-3 Nectin-4 cell lines); tissue samples (e.g., urothelial tissue, breast, ovaries, esophagus, bladder, peripheral blood mononuclear cells (PBMCs), leukocytes, bone marrow, thymus, tissue biopsy, tumor, lymph node, gut associated lymphoid tissue, mucosa associated lymphoid tissue, spleen, other lymphoid tissues, liver, lung, stomach, intestine, colon, kidney, pancreas, bone, prostate, cervix, testes, tonsil, or other organ, and/or cells derived therefrom); genetic expression components (e.g., genes for expression provided by vectors (e.g., lentiviral vector, retroviral vector), CRISPR components, ZFNs, TALENs, MegaTALs, targeted viral vectors and/or nanoparticles); cell formulation or activation components (e.g., saline, buffered saline, phosphate buffered saline (PBS); biocompatible buffers such as, Ca++/Mg++ free PBS; physiological saline, water, Hanks' solution, Ringer's solution, T-cell stimulating epitopes (e.g., anti-CD3/anti-CD28 conjugated beads; OKT3, TGN1412), culture-initiating compositions, RPMI, non-essential amino acids, sodium pyruvate, penicillin/streptomycin, non-dividing EBV-transformed lymphoblastoid cells (LCL), IL-21, human serum albumin (HSA) or other human serum components or fetal bovine serum, dextrose, stabilizers, preservatives); combination therapy components (e.g., local anesthetics, chemotherapeutic agents, immunosuppressive agents, anti-inflammatory agents); an antibody tagged with a fluorescent molecule; PCR amplification sequences; cytokines (e.g., IL-2, IL-7, IL-15, IL-21); culture vessels; reference levels; GAPDH; IFN-γ enzyme-linked immunosorbent assay (ELISA); culture plates; administration components (e.g., syringes, catheters, tubing, needles); etc.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (x) EXEMPLARY EMBODIMENTS

1. A chimeric antigen receptor (CAR) including, when expressed by a cell,
   an extracellular component including a Nectin-4 binding domain;
   an intracellular component including an effector domain; and
   a transmembrane domain linking the extracellular component to the intracellular component.
2. The CAR of embodiment 1, wherein the Nectin-4 binding domain has a complementarity determining region (CDR) set of antibody M22-321b41.1, according to North, Kabat, IMGT, Chothia, or Contact.
3. The CAR of embodiments 1 or 2, wherein the Nectin-4 binding domain includes a single chain variable fragment (scFv).
4. The CAR of embodiment 3, wherein the scFv has a variable heavy chain with at least 90% sequence identity to the sequence as set forth in SEQ ID NO: 57 and a variable light chain with at least 90% sequence identity to the sequence as set forth in SEQ ID NO: 58.
5. The CAR of embodiments 3 or 4, wherein the scFv has a variable heavy chain that has the sequence as set forth in SEQ ID NO: 57 and a variable light chain that has the sequence as set forth in SEQ ID NO: 58.
6. The CAR of any of embodiments 3-5, wherein the scFv has at least 90% sequence identity to the sequence as set forth in SEQ ID NO: 53 or SEQ ID NO: 55.
7. The CAR of any of embodiments 3-6, wherein the scFv has the sequence as set forth in SEQ ID NO: 53 or SEQ ID NO: 55.
8. The CAR of any of embodiments 3-7, wherein the scFv has a variable heavy chain that is encoded by a sequence with at least 90% sequence identity to the sequence as set forth in SEQ ID NO: 59 and a variable light chain that is encoded by a sequence with at least 90% sequence identity to the sequence as set forth in SEQ ID NO: 60.
9. The CAR of any of embodiments 3-8, wherein the scFv has a variable heavy chain that is encoded by the sequence as set forth in SEQ ID NO: 59 and a variable light chain that is encoded by the sequence as set forth in SEQ ID NO: 60.
10. The CAR of any of embodiments 3-9, wherein the scFv is encoded by a sequence having at least 90% sequence identity to the sequence as set forth in SEQ ID NO: 54 or SEQ ID NO: 56.
11. The CAR of any of embodiments 3-10, wherein the scFv is encoded by the sequence as set forth in SEQ ID NO: 54 or SEQ ID NO: 56.
12. The CAR of any of embodiments 1-11, wherein the extracellular component further includes a spacer.
13. The CAR of embodiment 12, wherein the spacer is 230 amino acids or less.
14. The CAR of embodiments 12 or 13, wherein the spacer includes the hinge region, CH2 domain, and CH3 domain of IgG4.
15. The CAR of embodiment 14, wherein the IgG4 is human IgG4.
16. The CAR of any of embodiments 12-15, wherein the spacer has at least 90% sequence identity to the sequence as set forth in SEQ ID NO: 23.
17. The CAR of any of embodiments 12-16, wherein the spacer has the sequence as set forth in SEQ ID NO: 23.
18. The CAR of any of embodiments 12-17, wherein the spacer is encoded by a sequence having at least 90% sequence identity to the sequence as set forth in SEQ ID NO: 24.
19. The CAR of any of embodiments 12-18, wherein the spacer is encoded by the sequence as set forth in SEQ ID NO: 24.
20. The CAR of embodiment 12, wherein the spacer is 160 amino acids or less.
21. The CAR of embodiments 12 or 20, wherein the spacer includes the hinge region and CH3 domain of IgG4.
22. The CAR of embodiment 21, wherein the IgG4 is human IgG4.
23. The CAR of any of embodiments 12-22, wherein the spacer has at least 90% sequence identity to the sequence as set forth in SEQ ID NO: 21.
24. The CAR of any of embodiments 12-23, wherein the spacer has the sequence as set forth in SEQ ID NO: 21.
25. The CAR of any of embodiments 12-24, wherein the spacer is encoded by a sequence having at least 90% sequence identity to the sequence as set forth in SEQ ID NO: 22.
26. The CAR of any of embodiments 12-25, wherein the spacer is encoded by the sequence as set forth in SEQ ID NO: 22.
27. The CAR of embodiment 12, wherein the spacer is 15 amino acids or less.
28. The CAR of embodiments 12 or 27, wherein the spacer includes the hinge region of IgG4.
29. The CAR of embodiment 28, wherein the IgG4 is human IgG4.
30. The CAR of any of embodiments 12-29, wherein the spacer has at least 90% sequence identity to the sequence as set forth in SEQ ID NO: 9.
31. The CAR of any of embodiments 12-30, wherein the spacer has the sequence as set forth in SEQ ID NO: 9.
32. The CAR of any of embodiments 12-31, wherein the spacer is encoded by a sequence having at least 90% sequence identity to the sequence as set forth in SEQ ID NO: 10, SEQ ID NO: 11, or SEQ ID NO: 12.
33. The CAR of any of embodiments 12-32, wherein the spacer is encoded by the sequence as set forth in SEQ ID NO: 10, SEQ ID NO: 11, or SEQ ID NO: 12.
34. The CAR of any of embodiments 1-33, wherein the effector domain includes all or a portion of the CD3ζ signaling domain; all or a portion of the 4-1BB signaling domain, all or a portion of the CD28 signaling domain, all or a portion of the CD3ζ signaling domain and the 4-1BB signaling domain; all or a portion of the CD3ζ signaling domain and all or a portion of the CD28 signaling domain; or all or a portion of the CD3ζ signaling domain, all or a portion of the 4-1BB signaling domain, and all or a portion of the CD28 signaling domain.
35. The CAR of embodiment 34, wherein the effector domain includes all or a portion of the CD3ζ signaling domain and all or a portion of the 4-1BB signaling domain.
36. The CAR of embodiments 34 or 35, wherein the effector domain includes all or a portion of the CD3ζ signaling domain, all or a portion of the 4-1BB signaling domain, and all or a portion of the CD28 signaling domain.
37. The CAR of any of embodiments 34-36, wherein the CD3ζ signaling domain has at least 90% sequence identity to the sequence as set forth in SEQ ID NO: 38, SEQ ID NO: 39, and/or SEQ ID NO: 40.
38. The CAR of any of embodiments 34-37, wherein the CD3ζ signaling domain has the sequence as set forth in SEQ ID NO: 38, SEQ ID NO: 39, or SEQ ID NO: 40.
39. The CAR of any of embodiments 34-38, wherein the CD3ζ signaling domain is encoded by a sequence having at least 90% sequence identity to the sequence as set forth in SEQ ID NO: 41 and/or SEQ ID NO: 42.
40. The CAR of any of embodiments 34-39, wherein the CD3ζ signaling domain is encoded by the sequence set forth in SEQ ID NO: 41 or SEQ ID NO: 42.
41. The CAR of any of embodiments 34-40, wherein the 4-1BB signaling domain has at least 90% sequence identity to the sequence as set forth in SEQ ID NO: 32, SEQ ID NO: 33, and/or SEQ ID NO: 34.
42. The CAR of any of embodiments 34-41, wherein the 4-1BB signaling domain has the sequence as set forth in SEQ ID NO: 32, SEQ ID NO: 33, or SEQ ID NO: 34.
43. The CAR of any of embodiments 34-42, wherein the 4-1BB signaling domain is encoded by a sequence having at least 90% sequence identity to the sequence as set forth in SEQ ID NO: 35, SEQ ID NO: 36, and/or SEQ ID NO: 37.
44. The CAR of any of embodiments 34-43, wherein the 4-1BB signaling domain is encoded by the sequence as set forth in SEQ ID NO: 35, SEQ ID NO: 36, or SEQ ID NO: 37.
45. The CAR of any of embodiments 34-44, wherein the CD28 signaling domain has at least 90% sequence identity to the sequence as set forth in SEQ ID NO: 110.
46. The CAR of any of embodiments 34-45, wherein the CD28 signaling domain has the sequence as set forth in SEQ ID NO: 110.
47. The CAR of any of embodiments 34-46, wherein the CD28 signaling domain is encoded by a sequence having at least 90% sequence identity to the sequence as set forth in SEQ ID NO: 111.
48. The CAR of any of embodiments 34-47, wherein the CD28 signaling domain is encoded by the sequence as set forth in SEQ ID NO: 111.
49. The CAR of embodiment 1-48, wherein the transmembrane domain includes a CD28 transmembrane domain.
50. The CAR of embodiment 49, wherein the CD28 transmembrane domain has at least 90% sequence identity to the sequence as set forth in SEQ ID NO: 25, SEQ ID NO: 26, and/or SEQ ID NO: 27.
51. The CAR of embodiments 49 or 50, wherein the CD28 transmembrane domain has the sequence as set forth in SEQ ID NO: 25, SEQ ID NO: 26, or SEQ ID NO: 27.
52. The CAR of any of embodiments 49-51, wherein the CD28 transmembrane domain is encoded by a sequence having at least 90% sequence identity to the sequence as set forth in SEQ ID NO: 28, SEQ ID NO: 29, SEQ ID NO: 117, and/or SEQ ID NO: 119.
53. The CAR of any of embodiments 49-52, wherein the CD28 transmembrane domain is encoded by the sequence as set forth in SEQ ID NO: 28, SEQ ID NO: 29, SEQ ID NO: 117, or SEQ ID NO: 119.
54. The CAR of any of embodiments 1-53, wherein the Nectin-4 binding domain includes a Nectin-4 scFv, the intracellular component includes the CD3ζ signaling domain and the 4-1BB signaling domain, and the transmembrane domain includes the CD28 transmembrane domain.
55. The CAR of any of embodiments 1-54, wherein the Nectin-4 binding domain includes a Nectin-4 scFv, the intracellular component includes the CD3ζ signaling domain, the 4-1BB signaling domain, and the CD28 signaling domain, and the transmembrane domain includes the CD28 transmembrane domain.
56. The CAR of any of embodiments 1-55, having at least 90% sequence identity to the sequence as set forth in SEQ ID NO: 2.
57. The CAR of any of embodiments 1-56, having the sequence as set forth in SEQ ID NO: 2.
58. The CAR of any of embodiments 1-57, encoded by a sequence having at least 90% sequence identity to the sequence as set forth in SEQ ID NO: 3.
59. The CAR of any of embodiments 1-58, encoded by the sequence as set forth in SEQ ID NO: 3.
60. The CAR of any of embodiments 1-59, further including a self-cleaving polypeptide.
61. The CAR of embodiment 60, wherein the self-cleaving polypeptide is a porcine teschovirus-1 (P2A), Thosea asigna virus (T2A), equine rhinitis A virus (E2A), foot-and-mouth disease virus (F2A), or variants thereof.
62. The CAR of embodiments 60 or 61, wherein the self-cleaving polypeptide is a T2A self-cleaving polypeptide.
63. The CAR of any of embodiments 1-62, further including a transduction marker.
64. The CAR of embodiment 63, wherein the transduction marker is a truncated epidermal growth factor receptor (EGFRt).
65. The CAR of embodiment 64, wherein the EGFRt has at least 90% sequence identity to the sequence as set forth in SEQ ID NO: 50.
66. The CAR of embodiments 64 or 65, wherein the EGFRt has the sequence as set forth in SEQ ID NO: 50.
67. The CAR of any of embodiments 64-66, wherein the EGFRt is encoded by a sequence having at least 90% sequence identity to the sequence as set forth in SEQ ID NO: 51.
68. The CAR of any of embodiments 64-67, wherein the EGFRt is encoded by the sequence as set forth in SEQ ID NO: 51.
69. The CAR of any of embodiments 1-68, further including a tag cassette or a suicide switch.
70. The CAR of any of embodiments 1-69, further including a multimerization domain.
71. A genetic construct encoding the CAR of any of embodiments 1-70.
72. The genetic construct of embodiment 71, wherein the genetic construct has at least 90% sequence identity to the sequence as set forth in SEQ ID NO: 1 and/or SEQ ID NO: 3.
73. The genetic construct of embodiments 71 or 72, wherein the genetic construct has the sequence as set forth in SEQ ID NO: 1 or SEQ ID NO: 3.
74. A nanoparticle encapsulating the genetic construct of any of embodiments 71-73.
75. A population of cells genetically modified to express the CAR of any of embodiments 1-70.
76. The population of cells of embodiment 75, wherein the population of cells includes autologous cells or allogeneic cells in reference to a subject.
77. The population of cells of embodiments 75 or 76, wherein the population is in vivo or ex vivo.
78. The population of cells of any of embodiments 75-77, wherein the population includes T-cells, B cells, natural killer (NK) cells, NK-T-cells, monocytes/macrophages, hematopoietic stem cells (HSC), and/or hematopoietic progenitor cell (HPCs).
79. The population of cells of any of embodiments 75-78, wherein the population includes CD4+ T-cells and CD8+ T-cells.
80. The population of cells of any of embodiments 75-79, wherein the population includes a 1:1 ratio of CD4+ T-cells to CD8+ T-cells.
81. A formulation including (i) cells genetically modified to express a CAR of any of embodiments 1-70 and (ii) a pharmaceutically acceptable carrier.
82. A method of providing an immune response against Nectin-4-expressing cells in a subject in need thereof including administering a therapeutically effective amount of the formulation of embodiment 81 to the subject thereby providing an immune response against Nectin-4-expressing cells in the subject.
83. The method of embodiment 82, wherein the Nectin-4-expressing cells are cancer cells.
84. The method of embodiments 82 or 83, wherein the immune response results in the killing of the cancer cells.
85. The method of embodiment 84, wherein the cancer cells are bladder cancer cells, breast cancer cells, ovarian cancer cells, esophageal cancer cells, lung cancer cells, colorectal cancer cells, head and neck squamous cell cancer cells, or pancreatic cancer cells.
86. The method of embodiment 85, wherein the bladder cancer cells are urothelial carcinoma cells.
87. The method of embodiment 85, wherein the breast cancer cells are triple-negative breast cancer (TNBC) cells or basal breast cancer cells.
88. The method of any of embodiments 82-87, wherein the administering a therapeutically effective amount includes administering intravesically, intravenously, intradermally, intraarterially, intraparenterally, intranodally, intralymphatically, intraperitoneally, intralesionally, intraprostatically, intravaginally, intrarectally, topically, intrathecally, intratumorally, intramuscularly, or subcutaneously.
89. The method of any of embodiments 82-88, wherein the administering a therapeutically effective amount includes administering intravesically.
90. The method of embodiment 89, wherein the administering intravesically includes instilling the formulation into the bladder of the subject for a select dwell time.
91. The method of embodiment 90, wherein the select dwell time includes 15 minutes to two hours.
92. The method of any of embodiments 89-91, wherein the method further includes emptying the bladder of the subject before instilling the formulation into the bladder of the subject.
93. The method of any of embodiments 89-92, wherein the method further includes administering an anticholinergic.

### (xi) CLOSING PARAGRAPHS

The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Functional variants include one or more residue additions or substitutions that do not substantially impact the physiological effects of the protein. Functional fragments include one or more deletions or truncations that do not substantially impact the physiological effects of the protein. A lack of substantial impact can be confirmed by observing experimentally comparable results in an activation study or a binding study. Functional variants and functional fragments of intracellular domains (e.g., intracellular signaling components) transmit activation or inhibition signals comparable to a wild-type reference when in the activated state of the current disclosure. Functional variants and functional fragments of binding domains bind their cognate antigen or ligand at a level comparable to a wild-type reference.

Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability of CAR disclosed herein to kill Nectin-4-expressing cells.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A chimeric antigen receptor (CAR) comprising, when expressed by a cell,
an extracellular component comprising a Nectin-4 binding domain;
an intracellular component comprising an effector domain; and
a transmembrane domain linking the extracellular component to the intracellular component.

2. The CAR of claim 1, wherein the Nectin-4 binding domain has a complementarity determining region (CDR) set of antibody M22-321b41.1, according to North, Kabat, IMGT, Chothia, or Contact.

3. The CAR of claim 1, wherein the Nectin-4 binding domain comprises a single chain variable fragment (scFv).

4. The CAR of claim 3, wherein the scFv has a variable heavy chain with at least 90% sequence identity to the sequence as set forth in SEQ ID NO: 57 and a variable light chain with at least 90% sequence identity to the sequence as set forth in SEQ ID NO: 58.

5. The CAR of claim 3, wherein the scFv has a variable heavy chain that has the sequence as set forth in SEQ ID NO: 57 and a variable light chain that has the sequence as set forth in SEQ ID NO: 58.

6. The CAR of claim 3, wherein the scFv has at least 90% sequence identity to the sequence as set forth in SEQ ID NO: 53 or SEQ ID NO: 55.

7. The CAR of claim 3, wherein the scFv has the sequence as set forth in SEQ ID NO: 53 or SEQ ID NO: 55.

8. The CAR of claim 3, wherein the scFv has a variable heavy chain that is encoded by a sequence with at least 90% sequence identity to the sequence as set forth in SEQ ID NO: 59 and a variable light chain that is encoded by a sequence with at least 90% sequence identity to the sequence as set forth in SEQ ID NO: 60.

9. The CAR of claim 3, wherein the scFv has a variable heavy chain that is encoded by the sequence as set forth in SEQ ID NO: 59 and a variable light chain that is encoded by the sequence as set forth in SEQ ID NO: 60.

10. The CAR of claim 3, wherein the scFv is encoded by a sequence having at least 90% sequence identity to the sequence as set forth in SEQ ID NO: 54 or SEQ ID NO: 56.

11. The CAR of claim 3, wherein the scFv is encoded by the sequence as set forth in SEQ ID NO: 54 or SEQ ID NO: 56.

12. The CAR of claim 1, wherein the extracellular component further comprises a spacer.

13. The CAR of claim 12, wherein the spacer is 230 amino acids or less.

14. The CAR of claim 12, wherein the spacer consists of a hinge region, CH2 domain, and CH3 domain of IgG4.

15. The CAR of claim 14, wherein the IgG4 is human IgG4.

16. The CAR of claim 12, wherein the spacer has at least 90% sequence identity to the sequence as set forth in SEQ ID NO: 23.

17. The CAR of claim 12, wherein the spacer has the sequence as set forth in SEQ ID NO: 23.

18. The CAR of claim 12, wherein the spacer is encoded by a sequence having at least 90% sequence identity to the sequence as set forth in SEQ ID NO: 24.

19. The CAR of claim 12, wherein the spacer is encoded by the sequence as set forth in SEQ ID NO: 24.

20. The CAR of claim 12, wherein the spacer is 160 amino acids or less.

21. The CAR of claim 12, wherein the spacer consists of a hinge region and CH3 domain of IgG4.

22. The CAR of claim 21, wherein the IgG4 is human IgG4.

23. The CAR of claim 12, wherein the spacer has at least 90% sequence identity to the sequence as set forth in SEQ ID NO: 21.

24. The CAR of claim 12, wherein the spacer has the sequence as set forth in SEQ ID NO: 21.

25. The CAR of claim 12, wherein the spacer is encoded by a sequence having at least 90% sequence identity to the sequence as set forth in SEQ ID NO: 22.

26. The CAR of claim 12, wherein the spacer is encoded by the sequence as set forth in SEQ ID NO: 22.

27. The CAR of claim 12, wherein the spacer is 15 amino acids or less.

28. The CAR of claim 12, wherein the spacer consists of the hinge region of IgG4.

29. The CAR of claim 28, wherein the IgG4 is human IgG4.

30. The CAR of claim 12, wherein the spacer has at least 90% sequence identity to the sequence as set forth in SEQ ID NO: 9.

31. The CAR of claim 12, wherein the spacer has the sequence as set forth in SEQ ID NO: 9.

32. The CAR of claim 12, wherein the spacer is encoded by a sequence having at least 90% sequence identity to the sequence as set forth in SEQ ID NO: 10, SEQ ID NO: 11, or SEQ ID NO: 12.

33. The CAR of claim 12, wherein the spacer is encoded by the sequence as set forth in SEQ ID NO: 10, SEQ ID NO: 11, or SEQ ID NO: 12.

34. The CAR of claim 1, wherein the effector domain comprises all or a portion of a CD3ζ signaling domain; all or a portion of a 4-1BB signaling domain; all or a portion of a CD28 signaling domain; all or a portion of the CD3ζ signaling domain and the 4-1BB signaling domain; all or a portion of the CD3ζ signaling domain and all or a portion of the CD28 signaling domain; or all or a portion of the CD3ζ signaling domain, all or a portion of the 4-1BB signaling domain, and all or a portion of the CD28 signaling domain.

35. The CAR of claim 34, wherein the effector domain comprises all or a portion of the CD3ζ signaling domain and all or a portion of the 4-1BB signaling domain.

36. The CAR of claim 34, wherein the effector domain comprises all or a portion of the CD3ζ signaling domain, all or a portion of the 4-1BB signaling domain, and all or a portion of the CD28 signaling domain.

37. The CAR of claim 34, wherein the CD3ζ signaling domain has at least 90% sequence identity to the sequence as set forth in SEQ ID NO: 38, SEQ ID NO: 39, and/or SEQ ID NO: 40.

38. The CAR of claim 34, wherein the CD3ζ signaling domain has the sequence as set forth in SEQ ID NO: 38, SEQ ID NO: 39, or SEQ ID NO: 40.

39. The CAR of claim 34, wherein the CD3ζ signaling domain is encoded by a sequence having at least 90% sequence identity to the sequence as set forth in SEQ ID NO: 41 and/or SEQ ID NO: 42.

40. The CAR of claim 34, wherein the CD3ζ signaling domain is encoded by the sequence set forth in SEQ ID NO: 41 or SEQ ID NO: 42.

41. The CAR of claim 34, wherein the 4-1BB signaling domain has at least 90% sequence identity to the sequence as set forth in SEQ ID NO: 32, SEQ ID NO: 33, and/or SEQ ID NO: 34.

42. The CAR of claim 34, wherein the 4-1BB signaling domain has the sequence as set forth in SEQ ID NO: 32, SEQ ID NO: 33, or SEQ ID NO: 34.

43. The CAR of claim 34, wherein the 4-1BB signaling domain is encoded by a sequence having at least 90% sequence identity to the sequence as set forth in SEQ ID NO: 35, SEQ ID NO: 36, and/or SEQ ID NO: 37.

44. The CAR of claim 34, wherein the 4-1BB signaling domain is encoded by the sequence as set forth in SEQ ID NO: 35, SEQ ID NO: 36, or SEQ ID NO: 37.

45. The CAR of claim 34, wherein the CD28 signaling domain has at least 90% sequence identity to the sequence as set forth in SEQ ID NO: 110.

46. The CAR of claim 34, wherein the CD28 signaling domain has the sequence as set forth in SEQ ID NO: 110.

47. The CAR of claim 34, wherein the CD28 signaling domain is encoded by a sequence having at least 90% sequence identity to the sequence as set forth in SEQ ID NO: 111.

48. The CAR of claim 34, wherein the CD28 signaling domain is encoded by the sequence as set forth in SEQ ID NO: 111.

49. The CAR of claim 1, wherein the transmembrane domain comprises a CD28 transmembrane domain.

50. The CAR of claim 49, wherein the CD28 transmembrane domain has at least 90% sequence identity to the sequence as set forth in SEQ ID NO: 25, SEQ ID NO: 26, and/or SEQ ID NO: 27.

51. The CAR of claim 49, wherein the CD28 transmembrane domain has the sequence as set forth in SEQ ID NO: 25, SEQ ID NO: 26, or SEQ ID NO: 27.

52. The CAR of claim 49, wherein the CD28 transmembrane domain is encoded by a sequence having at least 90% sequence identity to the sequence as set forth in SEQ ID NO: 28, SEQ ID NO: 29, SEQ ID NO: 117, and/or SEQ ID NO: 119.

53. The CAR of claim 49, wherein the CD28 transmembrane domain is encoded by the sequence as set forth in SEQ ID NO: 28, SEQ ID NO: 29, SEQ ID NO: 117, or SEQ ID NO: 119.

54. The CAR of claim 1, wherein the Nectin-4 binding domain comprises a Nectin-4 scFv, the intracellular component comprises the CD3ζ signaling domain and the 4-1BB signaling domain, and the transmembrane domain comprises the CD28 transmembrane domain.

55. The CAR of claim 1, wherein the Nectin-4 binding domain comprises a Nectin-4 scFv, the intracellular component comprises a CD3ζ signaling domain, a 4-1BB signaling domain, and a CD28 signaling domain, and the transmembrane domain comprises a CD28 transmembrane domain.

56. The CAR of claim 1, having at least 90% sequence identity to the sequence as set forth in SEQ ID NO: 2.

57. The CAR of claim 1, having the sequence as set forth in SEQ ID NO: 2.

58. The CAR of claim 1, encoded by a sequence having at least 90% sequence identity to the sequence as set forth in SEQ ID NO: 3.

59. The CAR of claim 1, encoded by the sequence as set forth in SEQ ID NO: 3.

60. The CAR of claim 1, further comprising a self-cleaving polypeptide.

61. The CAR of claim 60, wherein the self-cleaving polypeptide is a porcine teschovirus-1 (P2A), Thosea asigna virus (T2A), equine rhinitis A virus (E2A), foot-and-mouth disease virus (F2A), or variants thereof.

62. The CAR of claim 60, wherein the self-cleaving polypeptide is a T2A self-cleaving polypeptide.

63. The CAR of claim 1, further comprising a transduction marker.

64. The CAR of claim 63, wherein the transduction marker is a truncated epidermal growth factor receptor (EGFRt).

65. The CAR of claim 64, wherein the EGFRt has at least 90% sequence identity to the sequence as set forth in SEQ ID NO: 50.

66. The CAR of claim 64, wherein the EGFRt has the sequence as set forth in SEQ ID NO: 50.

67. The CAR of claim 64, wherein the EGFRt is encoded by a sequence having at least 90% sequence identity to the sequence as set forth in SEQ ID NO: 51.

68. The CAR of claim 64, wherein the EGFRt is encoded by the sequence as set forth in SEQ ID NO: 51.

69. The CAR of claim 1, further comprising a tag cassette or a suicide switch.

70. The CAR of claim 1, further comprising a multimerization domain.

71. A genetic construct encoding the CAR of claim 1.

72. The genetic construct of claim 71, wherein the genetic construct has at least 90% sequence identity to the sequence as set forth in SEQ ID NO: 1 and/or SEQ ID NO: 3.

73. The genetic construct of claim 71, wherein the genetic construct has the sequence as set forth in SEQ ID NO: 1 or SEQ ID NO: 3.

74. A nanoparticle encapsulating the genetic construct of claim 71.

75. A population of cells genetically modified to express the CAR of claim 1.

76. The population of cells of claim 75, wherein the population of cells comprises autologous cells or allogeneic cells in reference to a subject.

77. The population of cells of claim 75, wherein the population is in vivo or ex vivo.

78. The population of cells of claim 75, wherein the population comprises T-cells, B cells, natural killer (NK) cells, NK-T-cells, monocytes/macrophages, hematopoietic stem cells (HSC), and/or hematopoietic progenitor cell (HPCs).

79. The population of cells of claim 75, wherein the population comprises CD4+ T-cells and CD8+ T-cells.

80. The population of cells of claim 75, wherein the population comprises a 1:1 ratio of CD4+ T-cells to CD8+ T-cells.

81. A formulation comprising (i) cells genetically modified to express a CAR of claim 1 and (ii) a pharmaceutically acceptable carrier.

82. A method of providing an immune response against Nectin-4-expressing cells in a subject in need thereof comprising administering a therapeutically effective amount of the formulation of claim 81 to the subject thereby providing an immune response against Nectin-4-expressing cells in the subject.

83. The method of claim 82, wherein the Nectin-4-expressing cells are cancer cells.

84. The method of claim 82, wherein the immune response results in killing of the cancer cells.

85. The method of claim 84, wherein the cancer cells are bladder cancer cells, breast cancer cells, ovarian cancer cells, esophageal cancer cells, lung cancer cells, colorectal cancer cells, head and neck squamous cell cancer cells, or pancreatic cancer cells.

86. The method of claim 85, wherein the bladder cancer cells are urothelial carcinoma cells.

87. The method of claim 85, wherein the breast cancer cells are triple-negative breast cancer (TNBC) cells or basal breast cancer cells.

88. The method of claim 82, wherein the administering a therapeutically effective amount comprises administering intravesically, intravenously, intradermally, intraarterially, intraparenterally, intranodally, intralymphatically, intraperitoneally, intralesionally, intraprostatically, intravaginally, intrarectally, topically, intrathecally, intratumorally, intramuscularly, or subcutaneously.

89. The method of claim 82, wherein the administering a therapeutically effective amount comprises administering intravesically.

90. The method of claim 89, wherein the administering intravesically comprises instilling the formulation into the bladder of the subject for a select dwell time.

91. The method of claim 90, wherein the select dwell time comprises 15 minutes to two hours.

92. The method of claim 89, wherein the method further comprises emptying the bladder of the subject before instilling the formulation into the bladder of the subject.

93. The method of claim 89, wherein the method further comprises administering an anticholinergic.


SEQUENCE LISTING (123 sequences)

SEQ ID NO: 1 - DNA, 10413 bp, synthetic construct
  source 1..10413: mol_type = other DNA
  misc_feature 1087..1769: note = ColE1
  gene 2109..2131: gene = lacO
  misc_feature 2137..2157: note = M131-rev
  misc_feature 2175..2194: note = T3
  regulatory 2362..2890: note = CMV promoter; regulatory_class = promoter
  misc_feature 2891..2986: note = R
  misc_feature 2987..3071: note = U5
  misc_feature 3123..3260: note = PSI
  misc_feature 3725..3924: note = RRE
  misc_feature 4469..4648: note = cPPT
  misc_feature 5117..5136: note = pCCL-F
  misc_feature 4659..5194: note = MNDU3
  misc_feature 5180..5199: note = Primer CD19R F1
  misc_feature 5208..5213: note = EcoR1
  misc_feature 5220..5228: note = Kozak
  misc_feature 5229..5294: note = GM-CSF signal peptide
  V_region 5328..5666: note = M22-321b41.1 VL
  misc_feature 5667..5711: note = G4Sx3 Linker
  V_region 5712..6059: note = M22-321b41.1 VH
  misc_feature 6060..6095: note = IgG4 hinge
  misc_feature 6102..6110: note = 4/2N/Q-1
  misc_feature 6111..6421: note = IgG4-CH2
  misc_feature 6291..6293: note = 4/2NQ-2
  misc_feature 6421..6731: note = IgG4-CH3
  misc_feature 6744..6827: note = CD28tm
  misc_feature 6828..6953: note = 4-1BB
  misc_feature 6954..7298: note = CD3zeta
  misc_feature 7299..7370: note = T2A
  misc_feature 7371..8441: note = EGFRt
  misc_feature 8445..8450: note = Restored HpaI site
  misc_feature 8451..9038: note = dWPRE
  misc_feature 8809..8828: note = UTG-R
  regulatory 8845..8849: note = promoter mutation; regulatory_class = promoter
  regulatory 8860..8862: note = mutated start codon; regulatory_class = other
  misc_feature 9133..9185: note = U3
  misc_feature 9186..9281: note = R(1)
  misc_feature 9282..9366: note = U5(1)
  misc_feature 9766..9786: note = T7
  primer_bind 9795..9812: note = M13-fwd
  misc_feature 9883..9951: note = LacZ alpha
  regulatory 9956..10411: note = M13 origin; regulatory_class = other
  regulatory 9973..10279: note = F1 ori; regulatory_class = replication_regulatory_region
  misc_feature 330..989: note = AmpR
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagctaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggaattagcttgatcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatcgaattcgccaccgccgccaccatgctgctgctggtgaccagcctgctgctgtgcgagctgccccaccccgcctttctgctgatccccggatccgggggtgggagtggtgggagtacgcgtgatattgtaatgacgcaggcggcattttctaatccggttaccctcgggacctcagcttctatctcttgtagaagttccaaatccctgcttcattccaacggcattacttacctttattggtacctgcaaaaacctgggcagtcaccacaactcctgatatatcacatgtctaatttggcatctggggtccccgatcggtttacttcatcagggtccgggactgacttcacgctgcgcatctccagggttgaggcagaagatgttggcgtctactactgcgctcagaatttggaactcccgtttactttcggtggaggcactaagttggaaacaaagcgaggaggtggcggatctggtggaggagggagtggtggggggggttctcaagttcagttgcaacagtccggggcagagctggctcgaccaggtgcctccgtaaaactctcttgcaaagcatctggatatacctttacaacttactggatgcaatgggtaaaacagcgccccggtcagggacttgagtggataggctctatttatccaggcgacggagatactagatacacacagaagttcaaagggaaggcgacgctgacagctgataaaagctcaagcacagcatacattcaactgtccactcttgcgagtgaagacagtgccgtttattactgtgcgcgagaatactacgggctggattactggggccaaggtacgaccctcaccgtgtcatctgaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaagatgttctgggtgctggtggtggtgggcggggtgctggcctgctacagcctgctggtgacagtggccttcatcatcttttgggtaaagagaggacggaagaagctgctgtatatctttaagcagccttttatgagacctgtccaaacaacccaagaggaggacgggtgttcctgtcgcttcccagaggaggaggaaggagggtgcgagctgggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagttaacatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactgatcaaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 2 - protein, 657 aa, synthetic construct
  source 1..657: note = anti-Nectin4 scFv-IgG4Hinge-CH2-CH3-CD28TM-41BBSig-CD3zeta
DIVMTQAAFSNPVTLGTSASISCRSSKSLLHSNGITYLYWYLQKPGQSPQLLIYHMSNLASGVPDRFTSSGSGTDFTLRISRVEAEDVGVYYCAQNLELPFTFGGGTKLETKRGGGGSGGGGSGGGGSQVQLQQSGAELARPGASVKLSCKASGYTFTTYWMQWVKQRPGQGLEWIGSIYPGDGDTRYTQKFKGKATLTADKSSSTAYIQLSTLASEDSAVYYCAREYYGLDYWGQGTTLTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 1971 bp, synthetic construct
  source 1..1971: mol_type = other DNA; note = CAR with Nectin-4 binding domain encoding sequence (anti-Nectin4 scFv-IgG4Hinge-CH2-CH3-CD28TM-41BBSig-CD3zeta)
gatattgtaatgacgcaggcggcattttctaatccggttaccctcgggacctcagcttctatctcttgtagaagttccaaatccctgcttcattccaacggcattacttacctttattggtacctgcaaaaacctgggcagtcaccacaactcctgatatatcacatgtctaatttggcatctggggtccccgatcggtttacttcatcagggtccgggactgacttcacgctgcgcatctccagggttgaggcagaagatgttggcgtctactactgcgctcagaatttggaactcccgtttactttcggtggaggcactaagttggaaacaaagcgaggaggtggcggatctggtggaggagggagtggtggggggggttctcaagttcagttgcaacagtccggggcagagctggctcgaccaggtgcctccgtaaaactctcttgcaaagcatctggatatacctttacaacttactggatgcaatgggtaaaacagcgccccggtcagggacttgagtggataggctctatttatccaggcgacggagatactagatacacacagaagttcaaagggaaggcgacgctgacagctgataaaagctcaagcacagcatacattcaactgtccactcttgcgagtgaagacagtgccgtttattactgtgcgcgagaatactacgggctggattactggggccaaggtacgaccctcaccgtgtcatctgaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaagatgttctgggtgctggtggtggtgggcggggtgctggcctgctacagcctgctggtgacagtggccttcatcatcttttgggtaaagagaggacggaagaagctgctgtatatctttaagcagccttttatgagacctgtccaaacaacccaagaggaggacgggtgttcctgtcgcttcccagaggaggaggaaggagggtgcgagctgggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 4 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 5 - protein, 220 aa, synthetic construct
  source 1..220: note = AmpR
MSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 6 - protein, 66 aa, synthetic construct
  source 1..66: note = RRE
AVGIGALFLGFLGAAGSTMGAASMTLTVQARQLLSGIVQQQNNLLRAIEAQQHLLQLTVWGIKQLQ

SEQ ID NO: 7 - protein, 60 aa, synthetic construct
  source 1..60: note = cPPT; note = where X is a stop codon
HKWQYSSTILKEKGGLGGTVQGKEXXTXXQQTYKLKNYKNKLQKFKIFGFITGTAEIQFG

SEQ ID NO: 8 - protein, 22 aa, synthetic construct
  SIGNAL 1..22: note = GM-CSF signal peptide
MLLLVTSLLLCELPHPAFLLIP

SEQ ID NO: 9 - protein, 12 aa, synthetic construct
  source 1..12: note = IgG4 hinge-A
ESKYGPPCPPCP

SEQ ID NO: 10 - DNA, 36 bp, synthetic construct
  source 1..36: mol_type = other DNA; note = IgG4 hinge coding sequence-A
gaatctaagtacggaccgccctgccccccttgccct

SEQ ID NO: 11 - DNA, 36 bp, synthetic construct
  source 1..36: mol_type = other DNA; note = IgG4 hinge coding sequence-B
gagtctaagtacggaccgccttgcccaccgtgccca

SEQ ID NO: 12 - DNA, 36 bp, synthetic construct
  source 1..36: mol_type = other DNA; note = IgG4 hinge coding sequence
gagtctaagtacggaccgccctgccccccttgccct

SEQ ID NO: 13 - protein, 103 aa, synthetic construct
  DOMAIN 1..103: note = IgGr-CH2 domain A
GPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKA

SEQ ID NO: 14 - protein, 108 aa, synthetic construct
  DOMAIN 1..108: note = IgG4-CH2 domain -B
APPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKA

SEQ ID NO: 15 - DNA, 311 bp, synthetic construct
  source 1..311: mol_type = other DNA; note = IgG4-CH2 domain encoding sequence
ggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaa

SEQ ID NO: 16 - DNA, 326 bp, synthetic construct
  source 1..326: mol_type = other DNA; note = IgG4-CH2 domain encoding sequence
gccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaa

SEQ ID NO: 17 - protein, 104 aa, synthetic construct
  DOMAIN 1..104: note = IgG4-CH3 domain-A
KGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSL

SEQ ID NO: 18 - protein, 108 aa, synthetic construct
  DOMAIN 1..108: note = IgG4 CH3 domain-B
KGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 311 bp, synthetic construct
  source 1..311: mol_type = other DNA; note = IgG4-CH3 encoding sequence-A
agggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctg

SEQ ID NO: 20 - DNA, 323 bp, synthetic construct
  source 1..323: mol_type = other DNA; note = IgG4 CH3 domain coding sequence-B
agggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaag

SEQ ID NO: 21 - protein, 119 aa, synthetic construct
  source 1..119: note = Intermediate Spacer (IgG4 hinge-CH3 domain)
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - DNA, 357 bp, synthetic construct
  source 1..357: mol_type = other DNA; note = Intermediate Spacer encoding sequence (IgG4 hinge-CH3 domain)
gaatctaagtacggaccgccctgccccccttgccctggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaag

SEQ ID NO: 23 - protein, 224 aa, synthetic construct
  source 1..224: note = Long Spacer (IgG4 hinge-CH2 domain-CH3 domain)
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSL

SEQ ID NO: 24 - DNA, 672 bp, synthetic construct
  source 1..672: mol_type = other DNA; note = Long Spacer encoding sequence (IgG4 hinge-CH2 domain-CH3 domain)
gaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctg

SEQ ID NO: 25 - protein, 28 aa, synthetic construct
  DOMAIN 1..28: note = CD28 Transmembrane Domain-A
MFWVLVVVGGVLACYSLLVTVAFIIFWV

SEQ ID NO: 26 - protein, 27 aa, synthetic construct
  DOMAIN 1..27: note = CD28 Transmembrane Domain - B
MFWVLVVVGGVLACYSLLVTVAFIIFW

SEQ ID NO: 27 - protein, 26 aa, synthetic construct
  DOMAIN 1..26: note = CD28 Transmembrane Domain - C
FWVLVVVGGVLACYSLLVTVAFIIFW

SEQ ID NO: 28 - DNA, 84 bp, synthetic construct
  source 1..84: mol_type = other DNA; note = CD28TM encoding sequence-A
atgttctgggtgctggtggtggtgggcggggtgctggcctgctacagcctgctggtgacagtggccttcatcatcttttgggta

SEQ ID NO: 29 - DNA, 84 bp, synthetic construct
  source 1..84: mol_type = other DNA; note = CD28TM encoding sequence-B
atgttctgggtgctggtggtggtcggaggcgtgctggcctgctacagcctgctggtcaccgtggccttcatcatcttttgggtg

SEQ ID NO: 30-31 - skipped (2 entries)

SEQ ID NO: 32 - protein, 42 aa, synthetic construct
  DOMAIN 1..42: note = 41BB costimulatory domain -A
KRGRKKLLYIFKQPFMRPVQTTQEEDGCSCRFPEEEEGGCEL

SEQ ID NO: 33 - protein, 43 aa, synthetic construct
  DOMAIN 1..43: note = 4-1BB costimulatory domain -C
VKRGRKKLLYIFKQPFMRPVQTTQEEDGCSCRFPEEEEGGCEL

SEQ ID NO: 34 - protein, 41 aa, synthetic construct
  DOMAIN 1..41: note = 4-1BB costimulatory domain -C
VKRGRKKLLYIFKQPFMRPVQTTQEEDGCSCRFPEEEEGGC

SEQ ID NO: 35 - DNA, 126 bp, synthetic construct
  source 1..126: mol_type = other DNA; note = 4-1BB signaling encoding sequence-A
aagagaggacggaagaagctgctgtatatctttaagcagccttttatgagacctgtccaaacaacccaagaggaggacgggtgttcctgtcgcttcccagaggaggaggaaggagggtgcgagctg

SEQ ID NO: 36 - DNA, 126 bp, synthetic construct
  source 1..126: mol_type = other DNA; note = 4-1BB signaling encoding sequence-B
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 37 - DNA, 129 bp, synthetic construct
  source 1..129: mol_type = other DNA; note = 4-1BB signaling coding sequence-C
gtgaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 38 - protein, 115 aa, synthetic construct
  DOMAIN 1..115: note = CD3zeta signaling domain-A
GGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 112 aa, unidentified
  DOMAIN 1..112: note = CD3zeta signaling domain-B
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 114 aa, synthetic construct
  DOMAIN 1..114: note = CD3 zeta signaling domain -C
ELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - DNA, 345 bp, synthetic construct
  source 1..345: mol_type = other DNA; note = CD3zeta signaling domain encoding sequence-A
ggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 42 - DNA, 336 bp, synthetic construct
  source 1..336: mol_type = other DNA; note = CD3zeta signaling domain encoding sequence-B
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 43 - protein, 24 aa, Thosea asigna virus
  PEPTIDE 1..24: note = Thoseaasigna Virus 2A (T2A) Peptide-v1
LEGGGEGRGSLLTCGDVEENPGPR

SEQ ID NO: 44 - protein, 21 aa, Thosea asigna virus
  PEPTIDE 1..21: note = Thoseaasigna Virus 2A (T2A) Peptide-v2
GSGEGRGSLLTCGDVEENPGP

SEQ ID NO: 45 - protein, 22 aa, synthetic construct
  PEPTIDE 1..22: note = Porcine Teschovirus-1 2a (P2A) Peptide
GSGATNFSLLKQAGDVEENPGP

SEQ ID NO: 46 - protein, 23 aa, synthetic construct
  PEPTIDE 1..23: note = Equine Rhinitis A Virus (ERAV) 2A (E2A) Peptide
GSGQCTNYALLKLAGDVESNPGP

SEQ ID NO: 47 - protein, 25 aa, Foot-and-mouth disease virus
  PEPTIDE 1..25: note = Foot-And-Mouth Disease Virus 2A (F2A) Peptide
GSGVKQTLNFDLLKLAGDVESNPGP

SEQ ID NO: 48 - DNA, 72 bp, synthetic construct
  source 1..72: mol_type = other DNA; note = T2A encoding sequence-v1
ctcgagggcggcggagagggcagaggaagtcttctaacatgcggtgacgtggaggagaatcccggccctagg

SEQ ID NO: 49 - DNA, 72 bp, synthetic construct
  source 1..72: mol_type = other DNA; note = T2A encoding sequence-v2
ctcgagggcggcggagagggcagaggaagtcttctaacatgcggtgacgtggaggagaatccaggccctagg

SEQ ID NO: 50 - protein, 357 aa, synthetic construct
  source 1..357: note = EGFRt
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 51 - DNA, 1071 bp, synthetic construct
  source 1..1071: mol_type = other DNA; note = EGFRt encoding sequence
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 52 - skipped

SEQ ID NO: 53 - protein, 244 aa, synthetic construct
DIVMTQAAFSNPVTLGTSASISCRSSKSLLHSNGITYLYWYLQKPGQSPQLLIYHMSNLASGVPDRFTSSGSGTDFTLRISRVEAEDVGVYYCAQNLELPFTFGGGTKLETKRGGGGSGGGGSGGGGSQVQLQQSGAELARPGASVKLSCKASGYTFTTYWMQWVKQRPGQGLEWIGSIYPGDGDTRYTQKFKGKATLTADKSSSTAYIQLSTLASEDSAVYYCAREYYGLDYWGQGTTLTVSS

SEQ ID NO: 54 - protein, 732 aa, synthetic construct
GATATTGTAATGACGCAGGCGGCATTTTCTAATCCGGTTACCCTCGGGACCTCAGCTTCTATCTCTTGTAGAAGTTCCAAATCCCTGCTTCATTCCAACGGCATTACTTACCTTTATTGGTACCTGCAAAAACCTGGGCAGTCACCACAACTCCTGATATATCACATGTCTAATTTGGCATCTGGGGTCCCCGATCGGTTTACTTCATCAGGGTCCGGGACTGACTTCACGCTGCGCATCTCCAGGGTTGAGGCAGAAGATGTTGGCGTCTACTACTGCGCTCAGAATTTGGAACTCCCGTTTACTTTCGGTGGAGGCACTAAGTTGGAAACAAAGCGAGGAGGTGGCGGATCTGGTGGAGGAGGGAGTGGTGGGGGGGGTTCTCAAGTTCAGTTGCAACAGTCCGGGGCAGAGCTGGCTCGACCAGGTGCCTCCGTAAAACTCTCTTGCAAAGCATCTGGATATACCTTTACAACTTACTGGATGCAATGGGTAAAACAGCGCCCCGGTCAGGGACTTGAGTGGATAGGCTCTATTTATCCAGGCGACGGAGATACTAGATACACACAGAAGTTCAAAGGGAAGGCGACGCTGACAGCTGATAAAAGCTCAAGCACAGCATACATTCAACTGTCCACTCTTGCGAGTGAAGACAGTGCCGTTTATTACTGTGCGCGAGAATACTACGGGCTGGATTACTGGGGCCAAGGTACGACCCTCACCGTGTCATCT

SEQ ID NO: 55 - protein, 244 aa, synthetic construct
QVQLQQSGAELARPGASVKLSCKASGYTFTTYWMQWVKQRPGQGLEWIGSIYPGDGDTRYTQKFKGKATLTADKSSSTAYIQLSTLASEDSAVYYCAREYYGLDYWGQGTTLTVSSGGGGSGGGGSGGGGSDIVMTQAAFSNPVTLGTSASISCRSSKSLLHSNGITYLYWYLQKPGQSPQLLIYHMSNLASGVPDRFTSSGSGTDFTLRISRVEAEDVGVYYCAQNLELPFTFGGGTKLETKR

SEQ ID NO: 56 - protein, 732 aa, synthetic construct
CAAGTTCAGTTGCAACAGTCCGGGGCAGAGCTGGCTCGACCAGGTGCCTCCGTAAAACTCTCTTGCAAAGCATCTGGATATACCTTTACAACTTACTGGATGCAATGGGTAAAACAGCGCCCCGGTCAGGGACTTGAGTGGATAGGCTCTATTTATCCAGGCGACGGAGATACTAGATACACACAGAAGTTCAAAGGGAAGGCGACGCTGACAGCTGATAAAAGCTCAAGCACAGCATACATTCAACTGTCCACTCTTGCGAGTGAAGACAGTGCCGTTTATTACTGTGCGCGAGAATACTACGGGCTGGATTACTGGGGCCAAGGTACGACCCTCACCGTGTCATCTGGAGGTGGCGGATCTGGTGGAGGAGGGAGTGGTGGGGGGGGTTCTGATATTGTAATGACGCAGGCGGCATTTTCTAATCCGGTTACCCTCGGGACCTCAGCTTCTATCTCTTGTAGAAGTTCCAAATCCCTGCTTCATTCCAACGGCATTACTTACCTTTATTGGTACCTGCAAAAACCTGGGCAGTCACCACAACTCCTGATATATCACATGTCTAATTTGGCATCTGGGGTCCCCGATCGGTTTACTTCATCAGGGTCCGGGACTGACTTCACGCTGCGCATCTCCAGGGTTGAGGCAGAAGATGTTGGCGTCTACTACTGCGCTCAGAATTTGGAACTCCCGTTTACTTTCGGTGGAGGCACTAAGTTGGAAACAAAGCGA

SEQ ID NO: 57 - protein, 116 aa, synthetic construct
QVQLQQSGAELARPGASVKLSCKASGYTFTTYWMQWVKQRPGQGLEWIGSIYPGDGDTRYTQKFKGKATLTADKSSSTAYIQLSTLASEDSAVYYCAREYYGLDYWGQGTTLTVSS

SEQ ID NO: 58 - protein, 113 aa, synthetic construct
DIVMTQAAFSNPVTLGTSASISCRSSKSLLHSNGITYLYWYLQKPGQSPQLLIYHMSNLASGVPDRFTSSGSGTDFTLRISRVEAEDVGVYYCAQNLELPFTFGGGTKLETKR

SEQ ID NO: 59 - protein, 348 aa, synthetic construct
CAAGTTCAGTTGCAACAGTCCGGGGCAGAGCTGGCTCGACCAGGTGCCTCCGTAAAACTCTCTTGCAAAGCATCTGGATATACCTTTACAACTTACTGGATGCAATGGGTAAAACAGCGCCCCGGTCAGGGACTTGAGTGGATAGGCTCTATTTATCCAGGCGACGGAGATACTAGATACACACAGAAGTTCAAAGGGAAGGCGACGCTGACAGCTGATAAAAGCTCAAGCACAGCATACATTCAACTGTCCACTCTTGCGAGTGAAGACAGTGCCGTTTATTACTGTGCGCGAGAATACTACGGGCTGGATTACTGGGGCCAAGGTACGACCCTCACCGTGTCATCT

SEQ ID NO: 60 - protein, 339 aa, synthetic construct
GATATTGTAATGACGCAGGCGGCATTTTCTAATCCGGTTACCCTCGGGACCTCAGCTTCTATCTCTTGTAGAAGTTCCAAATCCCTGCTTCATTCCAACGGCATTACTTACCTTTATTGGTACCTGCAAAAACCTGGGCAGTCACCACAACTCCTGATATATCACATGTCTAATTTGGCATCTGGGGTCCCCGATCGGTTTACTTCATCAGGGTCCGGGACTGACTTCACGCTGCGCATCTCCAGGGTTGAGGCAGAAGATGTTGGCGTCTACTACTGCGCTCAGAATTTGGAACTCCCGTTTACTTTCGGTGGAGGCACTAAGTTGGAAACAAAGCGA

SEQ ID NO: 61 - protein, 13 aa, synthetic construct
  BINDING 1..13: note = Nectin-4 CDRH1
KASGYTFTTYWMQ

SEQ ID NO: 62 - protein, 10 aa, synthetic construct
  BINDING 1..10: note = Nectin-4-Binding CDR Sequences
SIYPGDGDTR

SEQ ID NO: 63 - protein, 9 aa, synthetic construct
  BINDING 1..9: note = Nectin-4-Binding CDR Sequence
AREYYGLDY

SEQ ID NO: 64 - protein, 16 aa, synthetic construct
RSSKSLLHSNGITYLY

SEQ ID NO: 65 - protein, 8 aa, synthetic construct
  BINDING 1..8: note = Nectin-4-Binding CDR Sequence
YHMSNLAS

SEQ ID NO: 66 - protein, 9 aa, synthetic construct
  BINDING 1..9: note = Nectin-4-Binding CDR Sequence
AQNLELPFT

SEQ ID NO: 67 - protein, 5 aa, synthetic construct
TYWMQ

SEQ ID NO: 68 - protein, 17 aa, synthetic construct
SIYPGDGDTRYTQKFKG

SEQ ID NO: 69 - protein, 7 aa, synthetic construct
EYYGLDY

SEQ ID NO: 70 - protein, 7 aa, synthetic construct
  BINDING 1..7: note = Nectin-4-Binding CDR Sequence
HMSNLAS

SEQ ID NO: 71 - protein, 8 aa, synthetic construct
  BINDING 1..8: note = Nectin-4-Binding CDR Sequence
GYTFTTYW

SEQ ID NO: 72 - protein, 8 aa, synthetic construct
  BINDING 1..8: note = Nectin-4-Binding CDR Sequence
IYPGDGDT

SEQ ID NO: 73 - protein, 11 aa, synthetic construct
  BINDING 1..11: note = Nectin-4-Binding CDR Sequence
KSLLHSNGITY

SEQ ID NO: 74 - protein, 7 aa, synthetic construct
  BINDING 1..7: note = Nectin-4-Binding CDR Sequence
GYTFTTY

SEQ ID NO: 75 - protein, 6 aa, synthetic construct
  BINDING 1..6: note = Nectin-4-Binding CDR Sequence
YPGDGD

SEQ ID NO: 76 - protein, 6 aa, synthetic construct
  BINDING 1..6: note = Nectin-4-Binding CDR Sequence
TTYWMQ

SEQ ID NO: 77 - protein, 13 aa, synthetic construct
  BINDING 1..13: note = Nectin-4-Binding CDR Sequence
WIGSIYPGDGDTR

SEQ ID NO: 78 - protein, 8 aa, synthetic construct
  BINDING 1..8: note = Nectin-4-Binding CDR Sequence
AREYYGLD

SEQ ID NO: 79 - protein, 7 aa, synthetic construct
  BINDING 1..7: note = Nectin-4-Binding CDR Sequence
ITYLYWY

SEQ ID NO: 80 - protein, 10 aa, synthetic construct
  BINDING 1..10: note = Nectin-4-Binding CDR Sequence
LLIYHMSNLA

SEQ ID NO: 81 - protein, 8 aa, synthetic construct
  BINDING 1..8: note = Nectin-4-Binding CDR Sequence
AQNLELPF

SEQ ID NO: 82 - protein, 55 aa, synthetic construct
  VARIANT 1..55: note = Up to 9 sets of GGGGS may be absent
GGGGSGGGGSGGGGSGGGGSGGGGSGGGGSGGGGSGGGGSGGGGSGGGGSGGGGS

SEQ ID NO: 83 - protein, 90 aa, synthetic construct
  VARIANT 1..90: note = up to nine sets of GGGS and/or GGGGS may be absent in the formula (Gly3Ser)n(Gly4Ser)n where n is an integer between 1 and 10
GGGSGGGGSGGGSGGGGSGGGSGGGGSGGGSGGGGSGGGSGGGGSGGGSGGGGSGGGSGGGGSGGGSGGGGSGGGSGGGGSGGGSGGGGS

SEQ ID NO: 84 - protein, 70 aa, synthetic construct
  VARIANT 1..70: note = up to 9 sets of GGGS and/or GGS may be absent in the formula (Gly3Ser)n(Gly2Ser)n where n is an integer from 1 to 10
GGGSGGSGGGSGGSGGGSGGSGGGSGGSGGGSGGSGGGSGGSGGGSGGSGGGSGGSGGGSGGSGGGSGGS

SEQ ID NO: 85 - protein, 90 aa, synthetic construct; identical to SEQ ID NO: 83
  VARIANT 1..90: note = Up to nine sets of GGGS may be absent in accordance with the formula (Gly3Ser)n(Gly4Ser)1 where n is an integer from 1 to 10

SEQ ID NO: 86 - protein, 20 aa, synthetic construct
GGGGSGGGGSGGGGSGGGGS

SEQ ID NO: 87 - protein, 15 aa, synthetic construct
GGGGSGGGGSGGGGS

SEQ ID NO: 88 - protein, 10 aa, synthetic construct
GGGGSGGGGS

SEQ ID NO: 89 - protein, 5 aa, synthetic construct
GGGGS

SEQ ID NO: 90 - protein, 8 aa, synthetic construct
GGGSGGGS

SEQ ID NO: 91 - protein, 4 aa, synthetic construct
GGGS

SEQ ID NO: 92 - protein, 6 aa, synthetic construct
GGSGGS

SEQ ID NO: 93 - protein, 11 aa, synthetic construct
GGSGGGSGGSG

SEQ ID NO: 94 - protein, 10 aa, synthetic construct
GGSGGGSGSG

SEQ ID NO: 95 - protein, 8 aa, synthetic construct
GGSGGGSG

SEQ ID NO: 96 - protein, 45 aa, synthetic construct
  VARIANT 1..45: note = up to eight of GGGGS may be absent
GGGGSGGGGSGGGGSGGGGSGGGGSGGGGSGGGGSGGGGSGGGGS

SEQ ID NO: 97 - protein, 50 aa, synthetic construct
  VARIANT 1..50: note = up to 8 of EAAAK may be absent
EAAAKEAAAKEAAAKEAAAKEAAAKEAAAKEAAAKEAAAKEAAAKEAAAK

SEQ ID NO: 98 - protein, 6 aa, synthetic construct
  source 1..6: note = His tag
HHHHHH

SEQ ID NO: 99 - protein, 8 aa, synthetic construct
  source 1..8: note = Flag tag
DYKDDDDK

SEQ ID NO: 100 - protein, 8 aa, synthetic construct
  source 1..8: note = Xpress tag
DLYDDDDK

SEQ ID NO: 101 - protein, 15 aa, synthetic construct
  source 1..15: note = Avi tag
GLNDIFEAQKIEWHE

SEQ ID NO: 102 - protein, 26 aa, synthetic construct
  source 1..26: note = Calmodulin tag
KRRWKKNFIAVSAANRFKKISSSGAL

SEQ ID NO: 103 - protein, 9 aa, synthetic construct
  source 1..9: note = HA tag
YPYDVPDYA

SEQ ID NO: 104 - protein, 10 aa, synthetic construct
  source 1..10: note = Myc tag
EQKLISEEDL

SEQ ID NO: 105 - protein, 8 aa, synthetic construct
  source 1..8: note = Strep tag
WRHPQFGG

SEQ ID NO: 106 - protein, 8 aa, synthetic construct
  source 1..8: note = STREP tag II
WSHPQFEK

SEQ ID NO: 107 - protein, 13 aa, synthetic construct
  source 1..13: note = STREP tag II
SLAELLNAGLGGS

SEQ ID NO: 108 - protein, 8 aa, synthetic construct
  source 1..8: note = Softag 3
TQDPSRVG

SEQ ID NO: 109 - protein, 14 aa, synthetic construct
  source 1..14: note = V5 tag
GKPIPNPLLGLDST

SEQ ID NO: 110 - protein, 1071 aa, synthetic construct
  source 1..1071: note = CAR with Nectin-4 binding domain, signal peptide, T2A, and EGFRt (GM-CSF signal peptide- anti-Nectin4 scFv-IgG4Hinge-CH2-CH3-CD28TM-41BBSig-CD3zeta-T2AEGFRt)
MLLLVTSLLLCELPHPAFLLIPGSGGGSGGSTRDIVMTQAAFSNPVTLGTSASISCRSSKSLLHSNGITYLYWYLQKPGQSPQLLIYHMSNLASGVPDRFTSSGSGTDFTLRISRVEAEDVGVYYCAQNLELPFTFGGGTKLETKRGGGGSGGGGSGGGGSQVQLQQSGAELARPGASVKLSCKASGYTFTTYWMQWVKQRPGQGLEWIGSIYPGDGDTRYTQKFKGKATLTADKSSSTAYIQLSTLASEDSAVYYCAREYYGLDYWGQGTTLTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 111 - DNA, 3215 bp, synthetic construct
  source 1..3215: mol_type = other DNA; note = CAR with Nectin-4 binding domain encoding sequence (GM-CSF signal peptide- anti- Nectin4 scFv-IgG4Hinge-CH2-CH3-CD28TM-41BBSig-CD3zeta-T2A-EGFRt)
atgctgctgctggtgaccagcctgctgctgtgcgagctgccccaccccgcctttctgctgatccccggatccgggggtgggagtggtgggagtacgcgtgatattgtaatgacgcaggcggcattttctaatccggttaccctcgggacctcagcttctatctcttgtagaagttccaaatccctgcttcattccaacggcattacttacctttattggtacctgcaaaaacctgggcagtcaccacaactcctgatatatcacatgtctaatttggcatctggggtccccgatcggtttacttcatcagggtccgggactgacttcacgctgcgcatctccagggttgaggcagaagatgttggcgtctactactgcgctcagaatttggaactcccgtttactttcggtggaggcactaagttggaaacaaagcgaggaggtggcggatctggtggaggagggagtggtggggggggttctcaagttcagttgcaacagtccggggcagagctggctcgaccaggtgcctccgtaaaactctcttgcaaagcatctggatatacctttacaacttactggatgcaatgggtaaaacagcgccccggtcagggacttgagtggataggctctatttatccaggcgacggagatactagatacacacagaagttcaaagggaaggcgacgctgacagctgataaaagctcaagcacagcatacattcaactgtccactcttgcgagtgaagacagtgccgtttattactgtgcgcgagaatactacgggctggattactggggccaaggtacgaccctcaccgtgtcatctgaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaagatgttctgggtgctggtggtggtgggcggggtgctggcctgctacagcctgctggtgacagtggccttcatcatcttttgggtaaagagaggacggaagaagctgctgtatatctttaagcagccttttatgagacctgtccaaacaacccaagaggaggacgggtgttcctgtcgcttcccagaggaggaggaaggagggtgcgagctgggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtg

SEQ ID NO: 112 - protein, 83 aa, synthetic construct
  DOMAIN 1..83: note = CD28/4-1BB signaling domain
RSKRSRGGHSDYMNMTPRRPGPTRKHYQPYAPPRDFAAYRSKRGRKKLLYIFKQPFMRPVQTTQEEDGCSCRFPEEEEGGCEL

SEQ ID NO: 113 - DNA, 249 bp, synthetic construct
  source 1..249: mol_type = other DNA
  CDS 1..249: note = CD28/4-1BB signaling domain coding sequence; protein_id = 112; translation = RSKRSRGGHSDYMNMTPRRPGPTRKHYQPYAPPRDFAAYRSKRG RKKLLYIFKQPFMRPVQTTQEEDGCSCRFPEEEEGGCEL
cggagcaagcggagcagaggcggccacagcgactacatgaacatgacccccagacggcctggccccacccggaagcactaccagccctacgccccacccagggactttgccgcctaccggtccaagagaggacggaagaagctgctgtatatctttaagcagccttttatgagacctgtccaaacaacccaagaggaggacgggtgttcctgtcgcttcccagaggaggaggaaggagggtgcgagctg

SEQ ID NO: 114 - skipped

SEQ ID NO: 115 - protein, 41 aa, synthetic construct
  DOMAIN 1..41: note = CD28 signaling domain
RSKRSRGGHSDYMNMTPRRPGPTRKHYQPYAPPRDFAAYRS

SEQ ID NO: 116 - protein, 123 aa, synthetic construct
  source 1..123: note = CD28 signaling domain coding sequence
CGGAGCAAGCGGAGCAGAGGCGGCCACAGCGACTACATGAACATGACCCCCAGACGGCCTGGCCCCACCCGGAAGCACTACCAGCCCTACGCCCCACCCAGGGACTTTGCCGCCTACCGGTCC

SEQ ID NO: 117 - DNA, 81 bp, synthetic construct
  source 1..81: mol_type = other DNA; note = CD28TM encoding sequence
  CDS 1..81: protein_id = 118; translation = MFWVLVVVGGVLACYSLLVTVAFIIFW
atgttctgggtgctggtggtggtcggaggcgtgctggcctgctacagcctgctggtcaccgtggccttcatcatcttttgg

SEQ ID NO: 118 - protein, 27 aa, synthetic construct
  source 1..27: note = CD28TM sequence-C
MFWVLVVVGGVLACYSLLVTVAFIIFW

SEQ ID NO: 119 - DNA, 81 bp, synthetic construct
  source 1..81: mol_type = other DNA; note = CD28TM encoding sequence
  CDS 1..81: protein_id = 120; translation = MFWVLVVVGGVLACYSLLVTVAFIIFW
atgttctgggtgctggtggtggtgggcggggtgctggcctgctacagcctgctggtgacagtggccttcatcatcttttgg

SEQ ID NO: 120 - protein, 27 aa, synthetic construct
  source 1..27: note = CD28TM sequence-D
MFWVLVVVGGVLACYSLLVTVAFIIFW

SEQ ID NO: 121 - DNA, 972 bp, synthetic construct
  source 1..972: mol_type = other DNA
  CDS 1..972: protein_id = 122; translation = MPPPRLLFFLLFLTPMEVRPEEPLVVKVEEGDNAVLQCLKGTSD GPTQQLTWSRESPLKPFLKLSLGLPGLGIHMRPLAIWLFIFNVSQQMGGFYLCQPGPP SEKAWQPGWTVNVEGSGELFRWNVSDLGGLGCGLKNRSSEGPSSPSGKLMSPKLYVWA KDRPEIWEGEPPCVPPRDSLNQSLSQDLTMAPGSTLWLSCGVPPDSVSRGPLSWTHVH PKGPKSLLSLELKDDRPARDMWVMETGLLLPRATAQDAGKYYCHRGNLTMSFHLEITA RPVLWHWLLRTGGWKVSAVTLAYLIFCLCSLVGILHLQRALVLRRKR
atgccacctccaagactcctcttcttcctcctcttcctgacaccaatggaagtcaggcctgaggaacctctagtggtgaaggtggaagagggagataacgctgtgttacagtgcctcaagggaacctcagatggacccactcagcagctgacctggtctcgggagtctccgcttaaacccttcctgaaactcagccttggactgccaggtctgggaatccacatgaggccactggctatctggctgttcatcttcaacgtctctcaacagatgggaggcttctacctgtgtcagcctggaccaccttctgagaaggcatggcagcctggttggacagtcaatgtggagggttctggtgagctgttccggtggaatgtttcggacctaggtggactgggatgtggtctgaagaacaggtcctcagagggacctagctctccttccgggaagctcatgagccccaagctgtatgtgtgggccaaagaccgccctgagatctgggagggagagcctccgtgtgtcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgccctgccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagatga

SEQ ID NO: 122 - protein, 323 aa, synthetic construct
  source 1..323: note = tCD19
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKR

SEQ ID NO: 123 - protein, 6 aa, synthetic construct
  source 1..6: note = polyglutamate tag
EEEEEE